SEQUENCE LISTING (4 sequences)

SEQ ID NO: 1 - protein, 51 aa, Homo Sapiens
MARYRCCRSQSRSRYYRQRQRSRRRRRRSCQTRRRAMRCCRPRYRPRCRRH

SEQ ID NO: 2 - DNA, 399 bp, Homo Sapiens
acagcccacagagttccacctgctcacaggttggctggctcagccaaggtggtgccctgctctgagcattcaggccaagcccatcctgcaccatggccaggtacagatgctgtcgcagccagagccggagcagatattaccgccagagacaaagaagtcgcagacgaaggaggcggagctgccagacacggaggagagccatgaggtgctgccgccccaggtacagaccgcgatgtagaagacactaattgcacaaaatagcacatccaccaaactcctgcctgagaatgttaccagacttcaagatcctcttgccacatcttgaaaatgccaccatccaataaaaatcaggagcctgctaaggaacaatgccgcctgtcaataaatgttgaaaagtca

SEQ ID NO: 3 - protein, 64 aa, Saccharolobus solfataricus
MATVKFKYKGEEKEVDISKIKKVWRVGKMISFTYDEGGGKTGRGAVSEKDAPKELLQMLEKQKK

SEQ ID NO: 4 - DNA, 195 bp, Saccharolobus solfataricus
atggcaacagtaaagttcaagtacaagggagaagagaaggaagtagatataagtaagataaagaaggtatggagagtaggcaaaatgataagtttcacctatgatgagggtggaggaaagactggtagaggagctgtaagcgagaaagacgctccaaaagaactactacaaatgttagaaaagcaaaagaaataa

(19) United States
(12) Patent Application Publication
(10) Pub. No.: US 2024/0084286 A1
(43) Pub. Date: Mar. 14, 2024

(54) METHODS AND COMPOSITIONS FOR INCREASING THE CONCENTRATION OF CELL FREE DNA

(71) Applicants: The Broad Institute, Inc., Cambridge, MA (US); Massachusetts Institute of Technology, Cambridge, MA (US); The General Hospital Corporation, Boston, MA (US)

(72) Inventors: Viktor A. Adalsteinsson, Cambridge, MA (US); Shervin Tabrizi, Cambridge, MA (US); Sangeeta N. Bhatia, Cambridge, MA (US); J. Christopher Love, Cambridge, MA (US); Maria Carmen Martin Alonso, Cambridge, MA (US); Kan Xiong, Cambridge, MA (US)

(73) Assignees: The Broad Institute, Inc., Cambridge, MA (US); Massachusetts Institute of Technology, Cambridge, MA (US); The General Hospital Corporation, Boston, MA (US)

(21) Appl. No.: 18/273,922

(22) PCT Filed: Jan. 25, 2022
(86) PCT No.: PCT/US2022/013769

§ 371 Date: Jul. 24, 2023

Related U.S. Application Data
(60) Provisional application No. 63/240,231, filed on Sep. 2, 2021.
(60) Provisional application No. 63/209,389, filed on Jun. 10, 2021.
(60) Provisional application No. 63/141,431, filed on Jan. 25, 2021.

(51) Int. Cl. C12N 15/10; A61K 9/51; A61K 38/00

(52) U.S. Cl. CPC C12N 15/1006; A61K 9/5123; A61K 38/005

(57) ABSTRACT

This disclosure provides a method for substantially increasing the concentration of cfDNA in a patient. By injecting a patient with lipid and/or polymer nanoparticles, agents that bind cfDNA, or inhibit deoxyribonucleases prior to collection of a sample of cfDNA, e.g., by way of a liquid biopsy, major pathways for the degradation of cfDNA are temporarily blocked, permitting transient accumulation of cfDNA. This strategy has the potential to dramatically enhance the quality of detection achieved by downstream cfDNA e analytical applications, such as sequencing applications.

### CROSS REFERENCE TO RELATED APPLICATIONS

This application claims the benefit under 35 U.S.C. § 119(e) of U.S. Provisional Application No. 63/141,431, filed Jan. 25, 2021, entitled “METHODS AND COMPOSITIONS FOR INCREASING THE CONCENTRATION OF CELL FREE DNA,” U.S. Provisional Application No. 63/209,389, filed Jun. 10, 2021, entitled “METHODS AND COMPOSITIONS FOR INCREASING THE CONCENTRATION OF CELL FREE DNA,” and U.S. Provisional Application No. 63/240,231, filed Sep. 2, 2021, entitled “METHODS AND COMPOSITIONS FOR INCREASING THE CONCENTRATION OF CELL FREE DNA,” the entire disclosures of each of which are hereby incorporated by reference in their entireties.

### GOVERNMENT SUPPORT

This invention was made with government support under grant number P30-CA14051 awarded by the National Institutes of Health. The government has certain rights in the invention.

### REFERENCE TO A SEQUENCE LISTING SUBMITTED AS A TEXT FILE VIA EFS-WEB

The present Application contains a Sequence Listing which has been submitted in ASCII format via EFS-Web and is hereby incorporated by reference in its entirety. The ASCII version, created on Jan. 25, 2022, is named B1195.70121WO00-SEQ and is 2,655 bytes in size.

### BACKGROUND

### Field of the Disclosure

The present disclosure relates to cell-free DNA (cfDNA). In particular, the disclosure relates to methods and compositions that enhance the isolation of cfDNA. Still further, the present disclosure relates to the use of agents, e.g., nanoparticles, agents capable of binding cfDNA, and agents capable of inhibiting deoxyribonucleases, to boost the concentration of cfDNA in a biological tissue to facilitate the detection, analysis, and/or measurement of cfDNA. The compositions, methods, and systems are applicable in the field of diagnostics (e.g., cancer screening), infectious disease diagnostics, prenatal testing, and other clinical screening and testing methodologies (i.e., not limited to cancer screening).

### Background of the Disclosure

Cancers are diverse and oftentimes difficult to treat. Prognosis is typically best when a cancer is detected early and when its genotype can be determined. For these reasons, liquid biopsies have generated tremendous interest for their ability to detect and profile cancer genomes using the “cell-free” DNA (cfDNA) present in a liquid sample from a patient. Blood biopsies are of particular interest. Unfortunately, analysis of cfDNA presents a major technical challenge. Most individuals possess very little cfDNA in their blood and the vast majority of cfDNA originates from healthy cells. Therefore, when the proportion of tumor-derived cfDNA fragments is very small relative to total cfDNA, sampling of a cancer genome during sequencing is sparse. This phenomenon is referred to as “limiting dilution” and impedes reliable cancer detection using cfDNA.

### SUMMARY OF THE INVENTION

The present disclosure describes methods, systems, and compositions for boosting the amount of cell-free DNA (cfDNA) in a biological specimen, and in particular, in the blood and/or urine, thereby improving analytical potential of cfDNA (which may include circulating tumor DNA or ctDNA) in the detection and/or diagnosis of a disease, e.g., cancer, and/or the analysis of biological information contained in the cfDNA (e.g., real-time treatment monitoring, tracking of drug resistance, detection of disease recurrence). Unfortunately, cfDNA is present in limited abundance in biological samples (e.g., blood and/or urine), and further, the subfraction of cfDNA that is actually derived from a diseased tissue (e.g., cfDNA derived from a tumor, e.g., ctDNA) is in even lower abundance and therefore difficult to test and/or analyze.

The low abundance of cfDNA, and consequently, disease-associated cfDNA (e.g., ctDNA), is also problematic when using liquid biopsies to conduct analyses. Liquid biopsies have garnered tremendous interest for their ability to detect and profile cancer genomes from the cfDNA in a liquid sample, such as a sample of blood. Major efforts are underway pursuing liquid biopsies for earlier detection of cancer, monitoring of treatment response, detection of minimal residual disease, tracking of cancer evolution, and selection of precision cancer therapy. However, most individuals have very little cfDNA in their blood, with most of it being derived from healthy cells rather than diseased cells, such as cancer cells. Thus, because the fraction of tumor-derived cfDNA fragments may be very low relative to total amount of cfDNA in a biological specimen, the specimen may comprise only a very sparse sampling of the cancer genome. This is what referred to as “limiting dilution” and it can be a major challenge for liquid biopsy testing. While most efforts to date have focused on increasing the analytical sensitivity of liquid biopsies (such as deeply sequencing more of the genome via error correction methods), no amount of sequencing can overcome physical absence of tumor DNA.

Accordingly, in various aspects and embodiments, the present disclosure provides methods, systems, and compositions for increasing and/or boosting the amount of cfDNA in a biological specimen, such as blood or urine. These methods, systems, and compositions involve techniques that alone or in combination may be used to boost the amount of cfDNA in a biological specimen, e.g., blood or urine. The techniques include: (1) inhibiting DNases in the tumor microenvironment to result in the increased amount of cfDNA (including ctDNA) in the blood; (2) increasing the half-life and/or stability of cfDNA in the blood, e.g., by administering cfDNA binders (e.g., DNA binding proteins, anti-DNA antibodies, DNA binding agents (e.g., protamine sulphate) that stabilize cfDNA in the blood and/or by limiting Kupffer cell phagocytosis of cfDNA in the liver by administering lipid and/or polymer nanoparticles in an amount effective to saturate the Kupffer cells, and optionally inhibiting DNases in the blood and/or in the Kupffer cells, thereby boosting the amount of cfDNA in the blood relative to the amount that would otherwise be present in the absence of such agents; and (3) channel cfDNA into the urine and optionally protect it from nuclease digestion by administering a DNA binding agent that induces renal clearance of the cfDNA. See Example 2 as an exemplary embodiment.

Accordingly, the present disclosure is based on various methods and compositions for boosting the levels of cell-free DNA (cfDNA) in a biological fluid (e.g., blood or urine) prior to the isolation of and/or assaying of the cfDNA. In various embodiments, the assaying step can include sequencing the cfDNA to detect one or more disease-associated genetic markers, e.g., cancer markers.

Another aspect of the present disclosure provides a method for increasing the concentration of cfDNA in a subject by administering a therapeutically effective amount of a nanoparticle to the subject that results in an increased concentration of cfDNA in one or more biological fluids of the subject. Without being bound by theory, the nanoparticles result in inhibiting Kupffer cell phagocytosis of cfDNA from the blood in the liver by becoming saturated with the administered nanoparticles.

In some embodiments, the method increases the concentration of cfDNA in the one or more biological fluids (e.g., blood) of the subject by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, as compared to the concentration of cfDNA in the blood in the absence of the nanoparticles.

In some embodiments, between 0.1 and 10 quadrillion nanoparticles are administered to the subject. In some embodiments, nanoparticles are administered to the subject at a rate of about 0.02 quadrillion nanoparticles per kilogram body weight of the subject.

In some embodiments, the biological fluids in which cfDNA concentration is increased are selected from blood, plasma, serum, lymph, synovial fluid, interstitial fluid, cerebrospinal fluid, urine, mucus, and saliva. In certain embodiments, the biological fluids comprise blood.

In some embodiments, the therapeutically effective amount of nanoparticle administered to the subject inhibits the activity of deoxyribonucleases in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the nanoparticle.

In some embodiments, the therapeutically effective amount of nanoparticle administered to the subject prevents the uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the nanoparticle.

In some embodiments, the nanoparticle comprises a naturally occurring lipid, a synthetic lipid, a naturally occurring polymer, or a synthetic polymer, or any combinations thereof.

In some embodiments, the nanoparticle comprises a naturally occurring or synthetic lipid selected from the following: phosphatidylglycerol (PG), phosphatidylcholine (PC), phosphatidylethanolamine (PEA), phosphatidylserine (PS), heptatriaconta-6,9,28,31-tetraen-19-yl 4-(dimethylamino)butanoate (DLin-MC3-DMA), cholesterol, 1,2-distearoyl-sn-glycero-3-phosphocholine (DSPC), 1,2-Distearoyl-sn-glycero-3-phosphoglycerol (DSPG), 1,2-dioleoyl-sn-glycero-3-phosphocholine (DOPC), 1,2-dierucoyl-sn-glycero-3-phosphocholine (DEPC), 1,2-dipalmitoyl-sn-glycero-3-phosphoglycerol (DPPG), 1,2-dioleoyl-sn-glycero-3-phosphoethanolamine (DOPE), 3-ethylphosphocholines (EPC), esterified propoxylated glycerol (EPG), 1,2-dimyristoyl-sn-glycero-3-phosphocholine (DMPC), 1,2-dioleoyl-3-trimethylammonium-propane (DOTAP), hydrogenated soy phosphatidylcholine (HSPC), and 1,2-dipalmitoyl-sn-glycero-3-phosphocholine (DPPC), or combinations thereof.

In some embodiments, the nanoparticle comprises a naturally occurring or synthetic polymer selected from the following: polyethylene glycol (PEG), poly-ε-caprolactone (PCL), poly(lactide-co-glycolide) (PLG), poly-L-lysine, alginate, a pollulan, and a cellulose, or combinations thereof.

In some embodiments, the nanoparticle encapsulates or is conjugated to an inert material. In certain embodiments, the inert material is colloidal gold, silver, or platinum.

In some embodiments, the nanoparticle is further conjugated to a deoxyribonuclease inhibitor. In certain embodiments, the deoxyribonuclease inhibitor is a DNase I inhibitor.

In some embodiments, the nanoparticle is further conjugated to a Kupffer cell-targeting moiety.

In some embodiments, the nanoparticle has a size distribution from 5 nanometers to 1000 nanometers in diameter.

In various embodiments, the nanoparticles comprise a population of nanoparticles having a size distribution that is homogenous. In other embodiments, the nanoparticles comprise a population of nanoparticles having a size distribution that is heterogenous. In various embodiments, at least 5% of the nanoparticles have approximately the same size diameter, at least 10% of the nanoparticles have approximately the same size diameter, at least 15% of the nanoparticles have approximately the same size diameter, at least 20% of the nanoparticles have approximately the same size diameter, at least 25% of the nanoparticles have approximately the same size diameter, or at least 30% of the nanoparticles have approximately the same size diameter, or at least 30% of the nanoparticles have approximately the same size diameter, or at least 35% of the nanoparticles have approximately the same size diameter, or at least 40% of the nanoparticles have approximately the same size diameter, or at least 45% of the nanoparticles have approximately the same size diameter, or at least 50% of the nanoparticles have approximately the same size diameter, or at least 55% of the nanoparticles have approximately the same size diameter, or at least 60% of the nanoparticles have approximately the same size diameter, or at least 65% of the nanoparticles have approximately the same size diameter, or at least 70% of the nanoparticles have approximately the same size diameter, or at least 75% of the nanoparticles have approximately the same size diameter, or at least 80% of the nanoparticles have approximately the same size diameter, or at least 85% of the nanoparticles have approximately the same size diameter, or at least 80% of the nanoparticles have approximately the same size diameter, or at least 95% of the nanoparticles have approximately the same size diameter, or at least 96% of the nanoparticles have approximately the same size diameter, or at least 97% of the nanoparticles have approximately the same size diameter, or at least 98% of the nanoparticles have approximately the same size diameter, or at least 99% of the nanoparticles have approximately the same size diameter, or up to 100% of the nanoparticles have approximately the same size diameter.

In some embodiments, the nanoparticles have a circulatory half-life of greater than 10 minutes. In other embodiments, the nanoparticles have a circulatory half-life of at least 2 minutes, or at least 3 minutes, or at least 4 minutes, or at least 5 minutes, or at least 6 minutes, or at least 7 minutes, or at least 8 minutes, or at least 9 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes, or at least 95 minutes, or at least 100 minutes, or at least 200 minutes, or at least 300 minutes, or at least 400 minutes, or at least 500 minutes, but not more than about 50, 150, 200, 250, 300, 350, 400, 450, 500, 550, 600, 650, 700, 750, 800, 850, 900, 950, or 1000, 1250, 1500, 1750, 2000, 2250, 2500, 2750, 3000, 3250, 3500, 2750, 4000, 4250, 4500, 4750, or 5000 minutes.

In some embodiments, the nanoparticle has a zeta potential from −70 mV to +70 mV. In some embodiments, the nanoparticle has a zeta potential from −60 mV to +60 mV. In various embodiments, the zeta potential is from about −200 mV to about +200 mV, or from about −200 mV to about +175 mV, or about −200 mV to about +150 mV, or about −200 mV to about +100 mV, or about −200 mV to about +75 mV, or about −200 mV to about +50 mV, or about −200 mV to about +25 mV, or about −200 mV to about +0 mV, or from about −175 mV to about +200 mV, or about −150 mV to about +200 mV, or about −125 mV to about +200 mV, or about −100 mV to about +200 mV, or about −75 mV to about +200 mV, or about −50 mV to about +200 mV, or about −25 mV to about +200 mV, or about 0 my to about +200 mV.

In some embodiments, the nanoparticle is administered intravenously. In other embodiments, the nanoparticles may be administered orally. In still other embodiments, the nanoparticles may be administered pulmonarily. In yet other embodiments, the nanoparticles may be administered transdermally. In other embodiments, the nanoparticles may be administered by multiple routes of administration, including, but not limited to, intravenously, orally, pulmonarily, and transdermally.

In some embodiments, the subject is a human patient. In some embodiments, the subject has, is suspected of having, or is at risk for a disease associated with the presence of cfDNA, e.g., cfDNA that is derived from a cancer cell or which comprises one or more disease-associated mutations. In some embodiments, the disease is cancer. In some embodiments, the cancer is colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma.

In another aspect, a method is provided for assaying cfDNA from a biological fluid by administering to the subject a therapeutically effective amount of a nanoparticle that results in an increased concentration of cfDNA in one or more biological fluids of the subject and then assaying the cfDNA. In some embodiments, the cfDNA is collected from the one or more biological fluids prior to assaying. In some embodiments, the one or more biological fluids includes blood.

In some embodiments, the assaying step of the method is conducted at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours, but not longer than 25 hours after administering the therapeutically effective amount of nanoparticle.

In some embodiments, the assaying step of the method comprises sequencing the cfDNA.

In some embodiments, the method further comprises identifying the subject as having a disease if the cfDNA from the sample is determined to contain mutations that are indicative of the disease. In some embodiments the disease is a cancer. In some embodiments, the cancer is colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma

In another aspect, a kit is provided for increasing the concentration of cfDNA in a subject having, suspected of having, or at risk for a disease associated with the presence of cfDNA from diseased tissue in one or more biological fluids, wherein the kit comprises a nanoparticle sufficient for increasing the concentration of cfDNA in one or more biological fluids of the subject when administered to a subject, a pharmacologically acceptable fluid in which the nanoparticle is stored, and instructions for use of the kit.

In another aspect, a method is provided for increasing the concentration of cfDNA in a subject by administering a therapeutically effective amount of an agent capable of binding cfDNA to the subject that results in an increased concentration of cfDNA in one or more biological fluids of the subject.

In some embodiments the method increases the concentration of cfDNA in the one or more biological fluids (e.g., blood) of the subject by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, as compared to the concentration of cfDNA in the blood in the absence of the agent capable of binding cfDNA.

In some embodiments, the biological fluids in which cfDNA concentration is increased are selected from blood, plasma, serum, lymph, synovial fluid, interstitial fluid, cerebrospinal fluid, urine, mucus, and saliva. In certain embodiments, the biological fluids comprise blood or urine.

In some embodiments, the therapeutically effective amount of the agent capable of binding cfDNA administered to the subject reduces the activity of deoxyribonucleases in the blood toward cfDNA of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the agent capable of binding cfDNA.

In some embodiments, the therapeutically effective amount of the agent capable of binding cfDNA administered to the subject prevents the uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the agent capable of binding cfDNA.

In some embodiments the agent capable of binding cfDNA comprises protamine, a Sso7d homolog, a monoclonal antibody specific for double stranded DNA (dsDNA), or a combination thereof. In some embodiments, the protamine is protamine sulphate. In some embodiments, the Sso7d homolog is from Sulfolobus solfataricus. In some embodiments, the monoclonal antibody specific for dsDNA is a single chain antibody.

In some embodiments, the agent capable of binding cfDNA is administered intravenously. In other embodiments, the agent may be administered orally. In still other embodiments, the agent may be administered pulmonarily. In yet other embodiments, the agent may be administered transdermally. In other embodiments, the agent may be administered by multiple routes of administration, including, but not limited to, intravenously, orally, pulmonarily, and transdermally.

In some embodiments, the subject is a human patient. In some embodiments, the subject has, is suspected of having, or is at risk for a disease associated with the presence of cfDNA, e.g., cfDNA that is derived from a cancer cell or which comprises one or more disease-associated mutations. In some embodiments, the disease is cancer. In some embodiments, the cancer is colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma.

In another aspect, a method is provided for increasing the concentration of cfDNA in a subject by administering a therapeutically effective amount of a nanoparticle, an agent capable of binding cfDNA, an agent capable of inhibiting the activity of deoxyribonucleases, or a combination thereof to the subject that results in an increased concentration of cfDNA in one or more biological fluids of the subject.

In some embodiments the method increases the concentration of cfDNA in the one or more biological fluids (e.g., blood) of the subject by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, as compared to the concentration of cfDNA in the blood in the absence of the agent capable of binding cfDNA.

In some embodiments, the biological fluids in which cfDNA concentration is increased are selected from blood, plasma, serum, lymph, synovial fluid, interstitial fluid, cerebrospinal fluid, urine, mucus, and saliva. In certain embodiments, the biological fluids comprise blood or urine.

In some embodiments, the therapeutically effective amount of the administration reduces the activity of deoxyribonucleases in the blood toward cfDNA of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of administration.

In some embodiments, the therapeutically effective amount of the administration prevents the uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the administration.

In some embodiments the nanoparticle is a nanoparticle specified above. In some embodiments the agent capable of binding cfDNA is an agent specified above. In some embodiments, the agent capable of inhibiting deoxyribonucleases comprises a DNase I inhibitor.

In some embodiments, the administration occurs intravenously. In other embodiments, administration occurs orally, pulmonarily, or transdermally. In other embodiments, the administration occurs by multiple routes, including, but not limited to, intravenously, orally, pulmonarily, and transdermally.

In some embodiments, the subject is a human patient. In some embodiments, the subject has, is suspected of having, or is at risk for a disease associated with the presence of cfDNA, e.g., cfDNA that is derived from a cancer cell or which comprises one or more disease-associated mutations. In some embodiments, the disease is cancer. In some embodiments, the cancer is colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma.

In another aspect, a method is provided for assaying cfDNA from a biological fluid by administering to the subject a therapeutically effective amount of a nanoparticle, an agent capable of binding cfDNA, an agent capable of inhibiting the activity of deoxyribonucleases, or a combination thereof that results in an increased concentration of cfDNA in one or more biological fluids of the subject and then assaying the cfDNA. In some embodiments, the cfDNA is collected from the one or more biological fluids prior to assaying. In some embodiments, the one or more biological fluids includes blood or urine.

In some embodiments, the assaying step of the method is conducted at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours, but not longer than 25 hours after administering the therapeutically effective amount of nanoparticle.

In some embodiments, the assaying step of the method comprises sequencing the cfDNA.

In some embodiments, the method further comprises identifying the subject as having a disease if the cfDNA from the sample is determined to contain mutations that are indicative of the disease. In some embodiments the disease is a cancer. In some embodiments, the cancer is colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma

In another aspect, a diagnostic composition is provided comprising a nanoparticle, an agent capable of binding cfDNA, and an agent capable of inhibiting the activity of deoxyribonucleases, or a combination thereof that results in an increased concentration of cfDNA in one or more biological fluids of a subject when administered to the subject. In some embodiments, the diagnostic composition further comprises a pharmacologically acceptable excipient.

In another aspect, a kit is provided for increasing the concentration of cfDNA in a subject having, suspected of having, or at risk for a disease associated with the presence of cfDNA from diseased tissue in one or more biological fluids, wherein the kit comprises a nanoparticle, an agent capable of binding cfDNA, an agent capable of inhibiting deoxyribonucleases, or a combination thereof sufficient for increasing the concentration of cfDNA in one or more biological fluids of the subject when administered to a subject, a pharmacologically acceptable fluid in which the nanoparticle is stored, and instructions for use of the kit.

The details of one or more embodiments of the present disclosure are set forth in the description below. Additional features or advantages of the present disclosure will be apparent from the following drawings and detailed description of several embodiments, as well as the appended claims.

### BRIEF DESCRIPTION OF DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 presents a schematic showing that intact nucleosomal cfDNA is both phagocytosed by Kupffer cells in the liver and digested by deoxyribonucleases (DNases) in the blood. After injection with nanoparticles, Kupffer cells become saturated with nanoparticles, limiting uptake of cfDNA, while DNases are prevented from digesting cfDNA.

FIG. 2 presents a schematic indicating one configuration in which nanoparticles enhance the amount of cfDNA recovered by a liquid biopsy (e.g., a blood sample) for use in downstream applications such as sequencing. By recovering an increased amount of cfDNA, sequencing more readily detects mutations in the subject which are indicative of disease. In step (a), the nanoparticles are administered in a therapeutically effective amount to a subject. The disclosure contemplates the use of any suitable nanoparticles as described herein, formulated in any suitable way, and administered by any suitable means (e.g., by injection). In one embodiment, the nanoparticles are administered in a therapeutically effective amount to the blood. The dosing can be in accordance with any suitable dosing scheme, as described herein. In addition, the administering of the nanoparticles can be performed as a single dose or multiple doses over a period of time. Step (b) represents the effect of the nanoparticles on the body. In particular, the nanoparticles prevent the degradation of cfDNA. Without being bound by theory, the nanoparticles prevent or otherwise reduce the DNase-dependent degradation of cfDNA in the blood. In addition, the nanoparticles prevent or otherwise reduce the degradation or otherwise removal of cfDNA by the Kupffer cells in the liver. Thus, step (b) represents a period of time whereby, due to the effects of the nanoparticles, the levels of cfDNA become boosted in the blood or other biological fluid. This “boosting” step can be allowed to progress for any suitable length of time, for instance, on the order of minutes to on the order of hours, days, or weeks. Next, once the levels of cfDNA are boosted in the biological fluid (e.g., blood), the biological fluid may be sampled, e.g., by withdrawing a small volume of blood using a needle, as shown in step (c). The sample can then be processed by any known means to isolate the cfDNA from the sample (e.g., any commercially available cfDNA isolation kit), as shown in step (d). The cfDNA is then sequenced in step (e) using any known means for sequencing (e.g., next-generation sequencing (NGS)) to obtained sequence data. The sequence data is then analyzed (e.g., computationally) to identify clinically relevant information, such as mutation information which correlate, predict, or otherwise which are associated with the prediction of a disease condition, or the risk of developing a disease condition.

FIGS. 3A-3B: FIG. 3A (top) presents a graph showing testing for 466 tumor mutations in serial dilutions of 20 ng sheared DNA (the amount of cfDNA in a typical 10 mL blood draw) reveals that many mutations are not physically present in the sample when tumor fraction is lower than ˜0.1%. FIG. 3A (bottom) presents a graph showing that increasing the mass of cfDNA drawn from the 1:100k dilution pool recovers the ‘lost’ signal. FIG. 3B presents a graph indicating that 18×biological replicates of 20 ng samples at 1:100k dilution shows stochastic sampling of tumor mutations.

FIGS. 4A-4B: FIG. 4A presents a graph showing cfDNA yields in healthy mice. FIG. 4B presents a graph showing cfDNA yields in tumor-bearing mice.

FIG. 5 presents graphs and tables depicting analysis of genome-wide tumor mutations isolated from cell-free DNA samples collected pre- vs. 1-hour post-injection with nanoparticles from tumor-bearing mice.

FIGS. 6A-6F. FIG. 6A presents the definition of genomic equivalent (GE) limit as the tumor fraction below which the cancer genome is not fully represented in a blood draw. The GE limit can be calculated by dividing 6.6E-3 ng (the mass of one haploid genome) by the ng of cfDNA sampled. FIG. 6B presents testing for 466 tumor mutations in serial dilutions of 20 ng cfDNA each, reveals that many mutations are not physically present in the sample when tumor fraction is below the GE limit. FIG. 6C presents 18 biological replicates of 20 ng samples at 1:100k dilution show stochastic representation of tumor mutations. FIG. 6D presents increasing masses of cfDNA drawn from the 1:100k dilution pool reveal restoration of tumor signal, suggesting that recovering more cfDNA could improve signal intensity. FIG. 6E presents tracking of individualized tumor mutation fingerprints in up to 20 ng of cfDNA from breast cancer patients who underwent curative intent treatment reveals early detection of recurrence in subset of patients, and the inset shows the number of mutations tracked versus detected in those cases. FIG. 6F presents GE limits calculated based on cfDNA yields for 1,323 blood samples from healthy donors and breast cancer patients.

FIG. 7 presents a schematic showing various contemplated approaches of the system disclosed herein for boosting the amount of cfDNA (including ctDNA) in the blood and/or urine in the non-limiting context where the cfDNA (including ctDNA) is produced from a tumor, including: (1) inhibiting DNases in the tumor microenvironment to result in the increased amount of cfDNA (including ctDNA) in the blood; (2) increasing the half-life and/or stability of cfDNA in the blood, e.g., by administering cfDNA binders (e.g., DNA binding proteins, anti-DNA antibodies, DNA binding agents (e.g., protamine sulphate) that stabilize cfDNA in the blood and/or by limiting Kupffer cell phagocytosis of cfDNA in the liver by administering lipid and/or polymer nanoparticles in an amount effective to saturate the Kupffer cells, and optionally inhibiting DNases in the blood and/or in the Kupffer cells, thereby boosting the amount of cfDNA in the blood relative to the amount that would otherwise be present in the absence of such agents; and (3) channel cfDNA into the urine and optionally protect it from nuclease digestion by administering a DNA binding agent that induces renal clearance of the cfDNA. See Example 2 as an exemplary embodiment.

FIGS. 8A-8B present the protection of dsDNA from nuclease digestion by various dsDNA binders. FIG. 8A presents a fluorescence assay measuring nuclease activity in digestion of dsDNA, which is diminished in presence of dsDNA binders. FIG. 8B presents a confirmation of dsDNA protection by Sso7d through digestion time-series of a dsDNA oligonucleotide.

FIG. 9 presents yeast surface display libraries of novel dsDNA binders. Three yeast surface display libraries of engineered single-domain antibodies demonstrating ability to bind to a dsDNA oligonucleotide.

FIGS. 10A-10B: FIG. 10A presents testing for 466 tumor mutations in serial dilutions of 20 ng cfDNA each reveals that many mutations are not physically present in the sample at low TF, and in biological replicates of 20 ng cfDNA samples at 1:100k dilution there is stochastic representation of tumor mutations. FIG. 10B presents increasing masses of cfDNA drawn from the 1:100k dilution pool reveal restoration of tumor signal, suggesting that recovering more cfDNA could improve signal intensity.

FIGS. 11A-11C: FIG. 11A presents a flow cytometry analysis of the dsDNA-binding nanobody library demonstrating clones with higher binding affinity. FIG. 11B presents a fluorescence assay demonstrating the ability of the small DBP Sso7d to protect dsDNA from DNase I digestion. FIG. 11C presents the RAW264.7 culture system demonstrating cellular uptake of Alexa488 labeled nucleosome core particles (NCP).

FIG. 12 presents a schematic showing the in vivo mechanism of action for liposomal contrast agents. 30 nm empty liposomes are administered intravenously 1 hour before blood collection. Liposomes saturate Kupffer cells (KCs), inhibiting the uptake of circulating free DNA (cfDNA) and increasing its accumulation in blood. Blood collected 1 hour post liposome treatment has a higher concentration of cfDNA and contains a higher number of mutations relative to blood pre-treatment.

FIGS. 13A-13D: FIG. 13A presents formulation and physical characteristics of liposomes synthesized. PBS stands for phosphate buffered saline and PE stands for phosphoethanolamine. FIG. 13B presents a schematic of an in vitro macrophage uptake inhibition assay. FIG. 13C presents representative images of uptake of Cy5-labelled nucleosome core particles (f-NCPs) by J774 (top) and RAW264 (bottom) cells after liposome pre-treatment for 4 hours. FIG. 13D presents quantification of f-NCP uptake via flow cytometry by J774 and RAW264 cells, respectively. % Cy5 positive cells was calculated based on the median-APC value, thresholding for cells above the cutoff set using the positive control (dashed vertical line).

FIGS. 14A-14C: FIG. 14A presents an experimental timeline to determine the blood bioavailability and the half-life of exoNCP in the absence or the presence of decoy liposomes. Mice were treated with liposomes or PBS intravenously (i.v.) 30 min. to 1 hour prior to i.v. administration of 1 μg of exoNCPs. Following, blood was sampled longitudinally, and plasma analyzed to determine the percentage of exoNCP remaining in plasma from the time of injection. FIG. 14B presents the percentage exoNCP remaining in plasma 1 hour after administration of different liposome doses (0, 100, 200, 300, 360 mg/kg, n=2-3). FIG. 14C presents plasma clearance of exoNCP 30 min. after the administration of liposomes (100 mg/kg) or PBS (n=3).

FIGS. 15A-15E: FIG. 15A presents an experimental timeline to detect mutations in the plasma of CT26 tumor-bearing mice in the absence or presence of the liposomal contrast agent. Blood was drawn prior to intravenous administration of PBS or liposomes at a range of liposome doses and plasma was collected 1 hour after treatment. FIG. 15B presents plasma DNA yield 1 hour after PBS or liposome treatment (n=5-7 per group; Mann-Whitney, two-tailed test on 1 hour post treatment DNA yields: PBS vs. 50 mg/kg p=0.0025, PBS vs. 100 mg/kg p=0.0025, PBS vs. 200 mg/kg p=0.0025, PBS vs. 300 mg/kg p=0.0079). FIG. 15C presents the number of mutant duplexes (MT) per milliliter of plasma (n=5-7 per group; Mann-Whitney, one-tailed test. PBS vs. 50 mg/kg p=0.009, PBS vs. 100 mg/kg p=0.015, PBS vs. 200 mg/kg p=0.015, PBS vs. 300 mg/kg p=0.009). FIG. 15D presents the number of sites detected for each treatment group as quantified using the custom-designed ctDNA assay. Mutational fingerprints are shown for one representative mouse per group. The top row represents mutations detected pre-treatment and the bottom row mutations detected 1-hour post-treatment in a given plasma sample. Each vertical band corresponds to a different site in a 98-site mutational panel and is shaded if detected at least once in the plasma sample. FIG. 15E presents the median tumor fractions detected pre-treatment and 1 hour post treatment for each treatment group as quantified using the custom-designed ctDNA assay.

FIG. 16 presents physical characterization of liposomes and fluorescently labeled liposomes.

FIGS. 17A-17B: FIG. 17A presents a time course of NCP digestion. NCPs were prepared by digesting the chromatin of CT26 cells for 5-30 min using a Nucleosome Preparation Kit (Active Motif). The NCPs were subjected to DNA cleanup and digestion efficiency assessed via electrophoresis through a 1.5% agarose gel. FIG. 17B presents labeling of NCPs with fluorescent dyes. NCPs were labelled with sulfo-Cy5 and Cy5 dyes (25 dye: 1 protein molar ratio, 4° C. at 550 rpm overnight) and labelling efficiency was measured as a fraction of Cy5 intensity to protein abundance via 4-12% Tris Glycine novex gels using native running buffer.

FIG. 18 presents an assessment of cell survival after treatment with liposomes. J774 cells were subjected to macrophage Cy5-NCP inhibition assays and stained using a live dead dye at end-point. Left column shows Cy5-NCP uptake for different conditions and right column the staining for live and dead cells. >99% of cells were alive after treatment, at each concentration tested.

FIG. 19A-19B: FIG. 19A presents organ biodistribution of Cy7-liposomes 1 hour after intravenous injection of liposomes (100 mg/kg, left) or PBS (right). FIG. 19B presents biodistribution quantified as a percent of total fluorescence across all organs.

FIG. 20 presents plasma clearance of Cy7-liposomes. Cy7-liposomes were administered intravenously (100 mg/kg liposomes, n=4) and plasma collected longitudinally over time. Cy7 fluorescence in undiluted plasma was quantified and plotted by normalizing all samples to the first plasma sample collected 5 min. after the liposome injection.

FIG. 21A-21B: FIG. 21A presents a schematic for the design of a CT26-specific tumor fingerprint panel. FIG. 21A presents validation of a ctDNA test designed for the CT26 bi-flank mouse model. The assay estimated tumor fractions are concordant with the actual tumor fractions down to 1:10000. Samples were prepared by serially diluting CT26 tumor gDNA into normal gDNA extracted from the buffy coat of healthy mice (three biological replicates were performed).

FIG. 22 presents a schematic for the processing of plasma samples to detect mutations. DNA is extracted from plasma samples and libraries constructed by ligation of corresponding adapters, CT26-specific SNVs are hybridized and captured, and sequencing is performed. Following alignment, the number of mutant and WT molecules can be detected and tumor fractions quantified for each SNV across the panel using previously published methods (Parsons et al. 2020).

FIG. 23 presents pre-treatment cfDNA yields for the tumor-bearing mice cohort. No statistical differences were observed between the pre-treatment DNA yields of PBS-treated or liposome-treated mice at any concentration (Mann-Whitney, two-tailed test. PBS vs. 50 mg/kg p=0.4318, PBS vs. 100 mg/kg p=0.1061, PBS vs. 200 mg/kg p=0.6389, PBS vs. 300 mg/kg p=0.1775).

FIG. 24 presents the number of sites from the 98-probe panel detected pre-treatment and 1-hour post-treatment for each mouse in the tumor-bearing mice cohort.

FIG. 25 presents a heatmap representation of the mutational fingerprints detected pre-treatment and 1-hour post-treatment for each mouse for the tumor-bearing mouse cohort. The top row represents mutations detected pretreatment and the bottom row mutations detected 1-hour post-treatment in a given plasma sample. Each vertical band corresponds to a different site in the 98-site mutational panel and is shaded if detected at least once in the plasma sample. Representative heatmaps for each group are shown surrounded by a box.

FIG. 26 presents cfDNA yields and tumor fractions for mice treated with 300 mg/kg liposomes and assessed until 3 hours after treatment with a CT26-ctDNA assay.

FIG. 27 presents a schematic showing the in vivo mechanism of cfDNA binders. cfDNA is normally rapidly cleared from the circulation via degradation by circulating nucleases and uptake by Kupffer cells in the liver, which results in low cfDNA concentration in plasma. An intravenously administered cfDNA binder protects cfDNA from degradation and clearance, temporarily boost enhancing the concentration of cfDNA in circulation. The increase of circulating cfDNA improves the sensitivity of cfDNA-based diagnostics from a blood draw, including detection of circulating tumor DNA (ctDNA).

FIGS. 28A-28E: FIG. 28A presents an experimental workflow for testing the effect of a DNA binding agent on clearance and degradation of cfDNA. A DNA-binding monoclonal antibody (mAb) is combined with recombinant nucleosome core particles (NCPs) consisting of a human histone octamer complexed with 20 ng of the Widom601 double-stranded DNA (dsDNA) oligonucleotide. The mixture is introduced into healthy BALB/c mice via tail vein injection and 70 μL blood-draws were obtained at 1 min., 15 min., and 60 min. post-injection. Levels of Widom601 were quantified via Taqman qPCR. FIG. 28B presents the amount of Widom601 remaining over time, normalized to the 1 min. blood draw, at various ratios of antibody to dsDNA (mg mAb/μg DNA). The greatest effect was observed at 2 mg/μg, with over 10-fold increase in the amount of Widom601 remaining compared to 0 mg/μg and IgG2a isotype control. FIG. 28C presents that treatment with anti-Fcγ receptor antibodies (anti-CD16, anti-CD32, anti-CD64) further augment Widom601 recovery at 60 min. Blockade of Fcγ receptors likely disrupts interaction of the Fc portion of the mAb with macrophages involved in clearance of immune complexes from the circulation and may be important for keeping this agent in circulation longer. FIG. 28D presents incubation of a DNA substrate with a fluorophore and quencher on either end with DNase I, either in the presence or absence of mAb. Digestion of the DNA leads to separation of fluorophore and quencher and detection of a fluorescent signal. In the presence of mAb, lower fluorescence signal is observed, consistent with protection of DNA from nuclease digestion. FIG. 28E presents an electropherogram of 147 bp Widom601 dsDNA in histones in the presence of DNase. In the presence of DNA-binding mAb, the DNA peak persists longer, consistent with protection of the dsDNA from nuclease digestion.

FIGS. 29A-29E: FIG. 29A presents an experimental workflow for testing the effect of a DNA binding agent on recovery of circulating tumor DNA (ctDNA). BALB/c mice bearing bilateral CT26 flank tumors are treated with the DNA-binding mAb. Retro-orbital blood draws are obtained prior to injection and 1 hour and 5 hours after injection. To detect ctDNA, cfDNA is tested for tumor mutations at pre-determined sites in the genome via hybrid-capture and deep sequencing. The total number of cfDNA fragments carrying tumor mutations (mutant duplexes) are reported. FIG. 29B presents data showing that treatment with the DNA binder increases the number of mutant duplexes detected per volume of plasma analyzed at 5 hours after injection. FIG. 29C presents data showing that treatment with DNA-binder does not reduce observed tumor fraction. FIG. 29D presents an experimental workflow similar to that of FIG. 29A, except for the inclusion of control mice injected with PBS. Blood samples in this experiment were terminal bleeds at 5 hours, which are approximately 10-fold higher in volume compared to blood draws in FIG. 29A. FIG. 29E presents data showing that treatment with 1 mg/kg DNA binder increases the number of mutant duplexes detected per volume of plasma analyzed at 5 hours after injection. FIG. 29F presents data showing that treatment with 1 mg/kg DNA-binder improves mutant duplex recovery without decreasing tumor fraction.

FIGS. 30A-30C: FIG. 30A presents the log fold-change of mean coverage across the genome from whole-genome sequencing of cfDNA from mice treated with DNA binder versus PBS control. Regions of the genome higher in GC content are consistently enriched following treatment with DNA binder. FIG. 30B presents data showing the increase in coverage following DNA binder treatment as a function of enrichment probe GC content across 98 120-bp probes used to capture regions bearing tumor mutations in cfDNA. Probes with the highest GC content had the greatest improvement in coverage with DNA binder treatment. Probe panels could be preferentially designed against enriched regions to further boost signal gain from DNA binder treatment. FIG. 30C presents data showing that treatment with DNA binder causes a characteristic shift in fragment size distribution of cfDNA toward shorter fragment sizes.

### DETAILED DESCRIPTION

The present disclosure provides methods, systems, and compositions for increasing and/or boosting the amount of cfDNA in a biological specimen, such as blood or urine. These methods, systems, and compositions relate to techniques that alone or in combination may be used to boost the amount of cfDNA in a biological specimen, e.g., blood or urine. Techniques described herein include, but are not limited to: (1) inhibiting DNases in the tumor microenvironment to result in the increased amount of cfDNA (including ctDNA) in the blood; (2) increasing the half-life and/or stability of cfDNA in the blood, e.g., by administering cfDNA binders (e.g., DNA binding proteins, anti-DNA antibodies, DNA binding agents (e.g., protamine sulphate) that stabilize cfDNA in the blood and/or by limiting Kupffer cell phagocytosis of cfDNA in the liver by administering lipid and/or polymer nanoparticles in an amount effective to saturate the Kupffer cells, and optionally inhibiting DNases in the blood and/or in the Kupffer cells, thereby boosting the amount of cfDNA in the blood relative to the amount that would otherwise be present in the absence of such agents; and (3) channel cfDNA into the urine and optionally protect it from nuclease digestion by administering a DNA binding agent that induces renal clearance of the cfDNA. See Example 2 as an exemplary embodiment.

Accurate detection of cell-free DNA (cfDNA) in liquid patient samples is challenging due to the relative scarcity of cfDNA, and in particular, disease-associated subfractions of cfDNA (e.g., ctDNA). This disclosure provides a method for substantially increasing the concentration of cfDNA in a patient. Surprisingly, by administering to a patient a lipid and/or polymer nanoparticles prior to collection of a sample comprising cfDNA, e.g., a sample of blood collected by liquid biopsy, major pathways for the degradation of cfDNA are temporarily blocked or otherwise inhibited, permitting transient accumulation of cfDNA. The methods may also include the administering of cfDNA binders and DNase inhibitors to block the degradation of cfDNA of interest. This approach resolves the underlying difficulties of cfDNA analysis by enhancing recovery of cfDNA upwards of 100-fold, dramatically enhancing the quality of detection achieved by downstream sequencing applications.

### Definitions

Unless otherwise defined herein, scientific and technical terms used in connection with the present disclosure shall have the meanings that are commonly understood by those of ordinary skill in the art (e.g., the skilled artisan). The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

General terminology in cell and molecular biology can be found in “The Merck Manual of Diagnosis and Therapy”, 19th Edition, published by Merck Research Laboratories, 2006 (ISBN 0-911910-19-0); Robert S. Porter et al. (Eds.), The Encyclopedia of Molecular Biology, published by Blackwell Science Ltd., 1994 (ISBN 0-632-02182-9). General terminology definitions in molecular biology are also given in Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

That the present disclosure may be more readily understood, select terms are defined below.

A an, and the

The singular forms “a”, “an”, and “the” include the plural unless the text clearly indicates otherwise. Similarly, the term “or” is intended to include “and” unless the text clearly indicates otherwise. Although methods and materials similar or equivalent to those described herein can be used in the practice or testing of the present disclosure, suitable methods and materials are described below. The abbreviation “e.g.,” is derived from the Latin phrase “exempli gratia” and is used herein to give a non-limiting example. For this reason, the abbreviation “for example (e.g.)” is synonymous with the term “for example”.

About

The terms “approximately” or “about,” as may be used interchangeably herein, and as applied to one or more values of interest, refer to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

Cancer

The term “cancer”, as may be used herein, refers to a cell or population of cells characterized by uncontrolled proliferation. The term “tumor”, as may be used herein, refers to a contiguous population of cancer cells. A cancer may be benign, meaning that it is localized to a single tissue, or malignant, meaning that it spreads to other parts of the body through the circulatory and/or lymphatic system. A cell or population of cells may be “pre-cancerous”, meaning that they share some characteristics of a cancer and risk developing into a cancer. Cells may become cancerous as a result of accumulated mutations in their genome. Examples of cancer include but are not limited to colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

Cell-Free DNA

The terms “cell-free DNA” or “cfDNA”, as may be used interchangeably herein, refer to deoxyribonucleic acid species that occur extracellularly. Cell-free DNA may originate from one or more cells. Cell-free DNA may originate from one or more cell types. Cell-free DNA may originate from healthy cells or diseased cells. Cell-free DNA may be single-stranded or double stranded. Cell-free DNA may interact with other species, such as histone proteins, to form higher order structures, such as nucleosomes. In some embodiments, cell-free DNA originates from the cells of a subject. In some embodiments, cell-free DNA originates from both healthy and diseased cells of a subject. In some embodiments, cell-free DNA encodes one or more genes belonging to the subject's genome. In some embodiments, cell-free DNA contains mutations that are indicative of a disease, such as a cancer.

Decrease

The terms “decrease”, “reduced”, “reduction”, or “inhibit” are all generally statistically significant herein. The terms are used to indicate a decrease in quantity, concentration, level, or the like. However, to prevent misunderstandings, “decreased”, “decreasing”, “decreasing”, or “inhibiting” is a reduction of at least 10% compared to the reference level, e.g., at least about 20% compared to the reference level. The % reduction may also be at least about 20%, or at least about 30%, or at least about 40%, or at least about 50%, or at least about 60%, or at least about 70%, or at least about 80%, or at least about 90%, or up to 100% reduction compared to the reference level. The reduction level may also be expressed in terms of fold-reduction, and includes at least a 2-fold reduction, or at least a 3-fold reduction, or at least a 3-fold reduction, or at least a 3-fold reduction, or at least a 3-fold reduction, or at least a 4-fold reduction, or at least a 5-fold reduction, or at least a 6-fold reduction, or at least a 7-fold reduction, or at least a 8-fold reduction, or at least a 9-fold reduction, or at least a 10-fold reduction, or at least a 11-fold reduction, or at least a 12-fold reduction, or at least a 13-fold reduction, or at least a 14-fold reduction, or at least a 15-fold reduction, or at least a 16-fold reduction, or at least a 17-fold reduction, or at least a 18-fold reduction, or at least a 19-fold reduction, or at least a 20-fold reduction, or at least a 25-fold reduction, or at least a 50-fold reduction, or more.

Increase

The terms “increased”, “increase”, “enhance” or “activate” are all generally statistically significant herein. The terms are used to indicate an increase in quantity, concentration, level, or the like. To prevent misunderstanding, the terms “increased”, “increase”, “enhance”, or “activate” is increase of at least 10% compared to the reference level, e.g., at least about 20% compared to the reference level. The % increase may also be at least about 20%, or at least about 30%, or at least about 40%, or at least about 50%, or at least about 60%, or at least about 70%, or at least about 80%, or at least about 90%, or up to 100% increase compared to the reference level. The increase level may also be expressed in terms of fold-increase, and includes at least a 2-fold increase, or at least a 3-fold increase, or at least a 3-fold increase, or at least a 3-fold increase, or at least a 3-fold increase, or at least a 4-fold increase, or at least a 5-fold increase, or at least a 6-fold increase, or at least a 7-fold increase, or at least a 8-fold increase, or at least a 9-fold increase, or at least a 10-fold increase, or at least a 11-fold increase, or at least a 12-fold increase, or at least a 13-fold increase, or at least a 14-fold increase, or at least a 15-fold increase, or at least a 16-fold increase, or at least a 17-fold increase, or at least a 18-fold increase, or at least a 19-fold increase, or at least a 20-fold increase, or at least a 25-fold increase, or at least a 50-fold increase, or more.

Isolated

The terms “isolated” or “partially purified” as used herein (e.g., in the context of isolated cfDNA) refers to a biological material (e.g., a cfDNA) that has been separated from other biological materials, e.g., from a biological sample of blood or other fluid.

Biological Sample

The term “biological sample” as used herein generally refers to a tissue or body fluid sample derived from a subject. Biological samples can be obtained directly from a subject. The biological sample can be or can comprise one or more nucleic acid molecules, such as deoxyribonucleic acid (DNA) or ribonucleic acid (RNA) molecules (e.g., cfDNA). The biological sample may be derived from any organ, tissue or biological fluid. The biological sample may comprise, for example, body fluids or solid tissue samples. An example of a solid tissue sample is a tumor sample, e.g., a solid tumor biopsy. Body fluids include, for example, blood, serum, plasma, tumor cells, saliva, urine, lymphatic fluid, synovial fluid, interstitial fluid, cerebrospinal fluid, prostate fluid, semen, sputum, mucus, gastric acid, bile, feces, tears, and derivatives thereof.

As used herein, the term “biological fluid” refers to any material occurring in a liquid phase that is naturally produced by cells of the subject and resides in one or more extracellular compartments. Biological fluids include, but are not limited to, blood, plasma, serum, lymph, synovial fluid, interstitial fluid, cerebrospinal fluid, urine, mucus, and saliva. A biological fluid may contain cells, as is the case for blood.

Liquid Biopsy

The term “liquid biopsy” as used herein generally refers to a non-invasive or minimally invasive laboratory test or assay (e.g., of a biological sample or of a cell-free DNA). Such a “liquid biopsy” assay may report a measurement of one or more tumor-associated marker genes (e.g., minor allele frequency, gene expression, or protein expression). For example, a circular tumor DNA test from Guardant Health, a Spotlight 59 oncology panel from Fluxion Biosciences, an Ultrasik from Agena Bioscience, such as the UltraSEEK lung cancer panel, the Foundation ACT fluid biopsy assay from Foundation Medicine, and the PlasmaSELECT assay from Personal Genome Diagnostics, are commercially available. Such assays may report a measure of the minor allele fraction (MAF) value for each set of genetic variants (e.g., single nucleotide variation (SNV), copy number variation (CNV), insertion/deletion (Indel), and/or fusion). The methods and compositions described herein for boosting the levels of cfDNA may be used in combination with a liquid biopsy to assay for the presence of a disease marker. In some embodiments, the liquid biopsy involves a non-invasive or minimally invasive laboratory test or assay on a sample of blood. In such cases, the liquid biopsy can be referred to as a “blood biopsy.”

Nanoparticle

As used herein, a “nanoparticle” is a small particle that ranges between about 1 to about 1000 nanometers in size. Undetectable by the human eye, nanoparticles can exhibit significantly different physical and chemical properties relative to their larger material counterparts. Nanoparticles can include, but are not limited to, liposomes, solid lipid nanoparticles, polymeric nanoparticles, dendrimer nanoparticles, silica nanoparticles, carbon nanoparticles, and magnetic nanoparticles. Nanoparticles may be constructed from naturally occurring materials, synthetic materials, or a combination of both.

Mutation

The term “mutation,” as may be used herein, refers to a change, alteration, or modification to a nucleotide in a nucleic acid (e.g., a cfDNA) as compared to its wild-type sequence. For example, without limitation, mutations may include substitutions, insertions, deletions, or any combination of the same in the cfDNA which is concentrated and then assayed by the methods described herein. In some embodiments, there is at least one mutation in cfDNA disclosed herein. In some embodiments, there is more than one mutation. In some embodiments, where there is more than one mutation, the mutations are distinct (e.g., not of the same type, e.g., substitutions, insertions, deletions). In some embodiments, where there is more than one mutation, the mutations are the same (e.g., the same type, e.g., substitutions, insertions, deletions). In some embodiments, the mutations result in a shifted reading frame (frameshift). In some embodiments, the mutations are indicative of a disease, such as a cancer. In other embodiments, the mutations are “disease-associated” mutations, which refers to mutations that predict that the subject in which the mutation exists has an increased chance or risk of having or developing a disease, e.g., cancer. Such disease-associate mutations may include point mutations wherein a single base is added, deleted or changed in a subject's DNA or RNA. The mutations may also be nonsense, missense, or silent mutations. A nonsense mutation occurs when one nucleotide is substituted, and this leads to the formation of a stop codon instead of a codon that encodes an amino acid. A stop codon is a certain sequence of bases (TAG, TAA, or TGA in DNA, and UAG, UAA, or UGA in RNA) that terminates the production of an amino acid chain during translation. Stop codons are always found at the end of a mRNA sequence when a protein is being produced, but if a substitution causes one to appear in another place, it will prematurely terminate the amino acid sequence and prevent the correct protein from being produced. Like a nonsense mutation, a missense mutation occurs when one nucleotide is substituted and a different codon is formed, but in this case the new codon encodes a different amino acid than was originally encoded. For example, if a missense substitution changes a codon from AAG to AGG, the amino acid arginine will be produced during translation instead of lysine. A mis sense mutation is considered conservative if the amino acid formed via the mutation has similar properties to the one that was originally encoded. It is non-conservative if the amino acid has different properties, which can disrupt the structure and function of a protein. A silent mutation is one in which a nucleotide is substituted but the mutation does not change the amino acid that a codon encodes. This can occur because multiple codons can code for the same amino acid. For example, AAG and AAA both code for lysine, so if the G is mutated to A, the same amino acid will be coded for during translation and the protein will not be affected. Even so, silent mutations can have deleterious effects, e.g., by altering mRNA stability. Disease-associated mutations may also include mutations that change the copy number of a gene by either duplicating or removing all or part of a gene in the genome. Disease-associated mutations may also include epigenetic modifications, such as changes in DNA methylation. DNA methylation refers to the addition of a methyl group to certain bases (C or A) in a nucleic acid molecule (e.g., changing C to 5-methylcytosine, mC). Changes in DNA methylation can occur within a coding portion of a gene (part of a gene that is transcribed and translated into protein) or within a non-coding portion of a gene (part of a gene that is transcribed and not translated into protein). Changes in DNA methylation can occur within the promotor sequence of a gene. Changes in the DNA methylation pattern of a gene can its expression level (i.e., cause it to be expressed at a higher or lower level than it is typically expressed).

Substantial)

The term “substantially,” as may be used herein, when used to describe the degree or abundance of an activity, generally refers to the value of the activity as being an amount which is achievable without undue effort. As can be appreciated, this amount may vary depending on the activity being performed, with simpler activities requiring a higher threshold and more complex activities requiring a lower threshold.

Subject

As used herein, “subject” means a human or animal. Usually, the animal is a vertebrate such as a primate, rodent, livestock animal, or hunting animal. Primates include chimpanzees, cynomolgus monkeys, spider monkeys, and macaques such as rhesus monkeys. Rodents include mice, rats, hamsters, rabbits, guinea pigs, squirrels, woodchucks, ferrets. Livestock and game animals include cattle, horses, pigs, deer, bison, buffalo, cat species such as domesticated cats, dog species such as domesticated dogs, foxes, wolves, birds such as chickens, turkeys, ducks, geese, emus, ostriches, and fish such as trout, catfish, and salmon. In some embodiments, the subject is a mammal, such as a primate, such as a human. The terms “individual”, “patient” and “subject” are used interchangeably herein. Preferably, the subject is a mammal. The mammal can be a human, non-human primate, mouse, rat, dog, cat, horse, or cow, but is not limited to these examples. Mammals other than humans can be conveniently used, for example, as subjects that represent animal models of cancer, e.g., a particular type of cancer, such as, lung cancer. The subject can be male or female. In various embodiments, the subject has or is at risk of having a disease state, such as cancer, and is in need of being evaluated, e.g., by a liquid biopsy, to test for the risk of having or developing a disease, e.g., cancer. In other embodiments, the subject has already been diagnosed or identified as having or being at risk of having a disease in need of treatment (e.g., cancer), or one or more complications associated with such diseases. In other embodiments, a subject has already been treated for a disease (e.g., cancer) or one or more complications associated with a disease, such as cancer. Alternatively, a subject can also be a subject that has not been previously diagnosed as having a disease (e.g., cancer) or one or more complications associated with the disease. For example, a subject can be a subject that exhibits one or more risk factors for a disease, or one or more complications associated with the disease (e.g., cancer), or a subject that does not exhibit a risk factor.

Subject in Need

A “subject in need” of a diagnosis and/or treatment for a particular condition (e.g., cancer) can be a subject who has a condition, has been diagnosed with a condition, or is at risk of developing the condition.

Therapeutically Effective Amount

A “therapeutically effective amount” refers to the amount of an agent (e.g., a nanoparticle or agent described herein) that is required to bring about an effect in a subject when administered to the subject. As used herein, the term “therapeutically effective amount” is synonymous with the term “therapeutically effective dose.” As used herein, a therapeutically effective amount does not require a clinically effective amount.

Nanoparticles and Methods of Making Same

The methods and kits described herein are based, at least in part, on the surprising discovery that nanoparticles were found to increase the overall concentration of cfDNA when administered to a subject.

In some embodiments, nanoparticles comprise particles that range between about 1 to about 1000 nanometers (i.e., 1 micrometer) in size. Nanoparticles embrace particles that can exhibit significantly different physical and chemical properties, including size, composition, charge, and shape. Nanoparticles can include, but are not limited to, liposomes, solid lipid nanoparticles, polymeric nanoparticles, dendrimer nanoparticles, silica nanoparticles, carbon nanoparticles, and magnetic nanoparticles. Nanoparticles may be constructed from naturally occurring materials, synthetic materials, or a combination of both.

In various embodiments, nanoparticles used herein may include any particle that measures no larger than 1000 nanometers (i.e., 1 micrometer) and no smaller than 1 nanometer across its longest cross-sectional dimension. Nanoparticles may be chemically homogenous, containing only a single distinct chemical species, or heterogenous, containing two or more distinct chemical species. Nanoparticles may be of any shape, including but not limited to spheres, ellipsoids, rods, tubes, cubes, and polyhedrons. Nanoparticles may be held together as stable entities either by covalent linkages between the chemical species of which they are composed, or by non-covalent forces, including but not limited to hydrophobic interactions. Nanoparticles may occur in a solid phase, meaning that they occur in a relatively stable arrangement of molecules, or in a fluid phase, in which the arrangement of molecules readily changes to some degree. Nanoparticles may be characterized by a particular zeta potential (i.e., electrokinetic potential).

In some embodiments, the nanoparticle has a longest cross-sectional dimension of less than 1 micrometer. For example, in some cases the nanoparticle can have a longest cross-sectional dimension of less than about 1 μm, less than about 900 nm, less than about 800 nm, less than about 700 nm, less than about 600 nm, less than about 500 nm, less than about 400 nm, less than about 300 nm, less than about 200 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm.

In some cases, a population of nanoparticles can be present. Various embodiments of the present disclosure are directed to such populations of nanoparticles. Populations of nanoparticles can be characterized as having a longest cross-sectional dimension that is on average less than about 1 μm, less than about 900 nm, less than about 800 nm, less than about 700 nm, less than about 600 nm, less than about 500 nm, less than about 400 nm, less than about 300 nm, less than about 200 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm. In some embodiments, a population of nanoparticles can be characterized as being substantially homogenous in terms of shape, size, and/or composition (i.e., “monodisperse”). In some embodiments, a population of nanoparticles can be distributed in the length of their longest cross-sectional dimension such that no more than about 10% or about 5% of nanoparticles have a longest cross-sectional dimension that is about 10% greater or less than the average characteristic dimension of the population of nanoparticles, and in some cases, such that no more than about 8%, about 5%, about 3%, about 1%, about 0.3%, about 0.1%, about 0.03%, or about 0.01% have a longest cross-sectional dimension that is about 10% greater or less than the average longest cross-sectional dimension of population of nanoparticles. The size distribution of a population of nanoparticles is readily determined by any method known in the art, such as dynamic light scattering. It is often desirable to produce a population of nanoparticles that is relatively uniform in terms of size, shape, and/or composition so that each nanoparticle has similar properties. In some embodiments, however, a population of nanoparticles can be heterogeneous with respect to size, shape, and/or composition.

In some embodiments, the nanoparticles are approximately spherical in shape, therefore the term “longest cross-sectional dimension” in the context of spherical nanoparticles is equivalent to the term “diameter”. In some embodiments, the diameter of no more than 25%, no more than 20%, no more than 15%, no more than 10%, or no more than 5% of nanoparticles in a population varies from the mean diameter of the population by more than 200%, 175%, 150%, 100%, 75%, 50%, 25%, 20%, 10%, or 5%.

In some embodiments, a population of nanoparticles can be characterized by their dispersity (i.e., polydispersity index). In some embodiments, a population of nanoparticles has a dispersity of 0.6 or less, 0.5 or less, 0.4 or less, 0.3 or less, 0.2 or less, 0.1 or less, or 0.05 or less.

In some embodiments, nanoparticles are composed of lipids, polymers, or a combination thereof. The term “lipid” or “lipids” as used herein refers to hydrophobic chemical species, including but not limited to oils, fats, fatty acids, fatty acid esters, waxes, sterols, and steroids. Lipids may be amphipathic, including but not limited to phospholipids. Lipids can be naturally occurring (i.e., found in nature) or synthetic (i.e., synthesized strictly through human processes). The term “polymer” refers to a chemical species that is composed of a defined set of one or more repeating chemical subunits that are covalently linked together as a single molecule. Repeating chemical subunits may be covalently linked in an order that is regularly repeating, in an order that is random, or in an order that is semi-random. Polymers may be naturally occurring (i.e., found in nature) or synthetic (i.e., synthesized strictly through human processes). In some embodiments, polymers are entirely or partially hydrophobic.

In some embodiments, a nanoparticle comprises at least one lipid and/or polymer layer that encloses an aqueous core. In some embodiments, a liposome has either a single lipid and/or polymer layer (a monolayer) or more than one lipid and/or polymer layer (a multilayer). In some embodiments, a liposome has two lipid and/or polymer layers (a bilayer). In some embodiments, the lipids and polymers from which one or more lipid and/or polymer layers are comprised are amphipathic.

A nanoparticle that is primarily composed of lipids, polymers, or a combination thereof, that comprises a bilayer enclosing an aqueous core is herein referred to as either a “lipid nanoparticle” or “liposome”. A nanoparticle that is primarily composed of lipids, polymers, or a combination thereof, that comprises a monolayer enclosing a solid lipophilic core is herein referred to as a “solid lipid nanoparticle”. A nanoparticle that is primarily composed of lipids, polymers, or a combination thereof, that comprises a monolayer without a core is herein referred to as a “micelle”.

In some embodiments, a nanoparticle comprises more than one distinct type of lipid or polymer. In some embodiments, a nanoparticle or liposome is composed of two lipids or polymers comprising a first lipid or polymer and a second lipid or polymer in at least a 1:1 ratio, at least a 1:2 ratio, at least a 1:3 ratio, at least a 1:4 ratio, at least a 1:5 ratio, at least a 1:6 ratio, at least a 1:7 ratio, at least a 1:8 ratio, at least a 1:9 ratio, at least a 1:10 ratio, at least a 1:15 ratio, at least a 1:20 ratio, at least a 1:25 ratio, at least a 1:50 ratio, at least a 1:100 ratio, at least a 1:250 ratio, at least a 1:500 ratio, or at least a 1:1000 ratio. In some embodiments, a nanoparticle is composed of three or more lipids or polymers wherein each possible pairing of a first lipid or polymer and a second lipid or polymer within the three or more lipids or polymers occur in at least a 1:1 ratio, at least a 1:2 ratio, at least a 1:3 ratio, at least a 1:4 ratio, at least a 1:5 ratio, at least a 1:6 ratio, at least a 1:7 ratio, at least a 1:8 ratio, at least a 1:9 ratio, at least a 1:10 ratio, at least a 1:15 ratio, at least a 1:20 ratio, at least a 1:25 ratio, at least a 1:50 ratio, at least a 1:100 ratio, at least a 1:250 ratio, at least a 1:500 ratio, or at least a 1:1000 ratio.

In some embodiments, a nanoparticle includes lipids selected from, but not limited to, one or more of the following classifications: fatty acids, glycerolipids, glycerophospholipids, sphingolipids, sterols, prenols, saccharolipids, polyketides.

In some embodiments, a nanoparticle comprises, though is not limited to, any of the following clinically relevant lipids: phosphatidylglycerol (PG), phosphatidylcholine (PC), phosphatidylethanolamine (PEA), phosphatidylserine (PS), heptatriaconta-6,9,28,31-tetraen-19-yl 4-(dimethylamino)butanoate (DLin-MC3-DMA), cholesterol, 1,2-distearoyl-sn-glycero-3-phosphocholine (DSPC), 1,2-Distearoyl-sn-glycero-3-phosphoglycerol (DSPG), 1,2-dioleoyl-sn-glycero-3-phosphocholine (DOPC), 1,2-dierucoyl-sn-glycero-3-phosphocholine (DEPC), 1,2-dipalmitoyl-sn-glycero-3-phosphoglycerol (DPPG), 1,2-dioleoyl-sn-glycero-3-phosphoethanolamine (DOPE), 3-ethylphosphocholines (EPC), esterified propoxylated glycerol (EPG), 1,2-dimyristoyl-sn-glycero-3-phosphocholine (DMPC), 1,2-dioleoyl-3-trimethylammonium-propane (DOTAP), hydrogenated soy phosphatidylcholine (HSPC), 1,2-dipalmitoyl-sn-glycero-3-phosphocholine (DPPC), or derivatives thereof.

In some embodiments, a nanoparticle includes polymers selected from, but not limited to, one or more of the following classifications: polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, and polyamines.

In some embodiments, a nanoparticle or liposome comprises, though is not limited to, any of the following clinically relevant polymers: polyethylene glycol (PEG), poly-ε-caprolactone (PCL), poly(lactide-co-glycolide) (PLG), poly-L-lysine, alginate, pollulans, and celluloses, or derivatives thereof.

In some embodiments, nanoparticles can be produced such that they are biodegradable. Biodegradable nanoparticles are composed of materials already approved by government regulatory agencies as such, and/or such that they result in a submicron size (e.g., 1 μm-10 nm, or other ranges, e.g., 500 nm-10 nm, 250 nm-25 nm, 100 nm-50 nm).

In some embodiments, the nanoparticle comprises a naturally occurring or synthetic polymer selected from the following: polyethylene glycol (PEG), poly-ε-caprolactone (PCL), poly(lactide-co-glycolide) (PLG), poly-L-lysine, alginate, a pollulan, and a cellulose, or combinations thereof.

In some embodiments, the nanoparticle encapsulates or is conjugated to an inert material. In certain embodiments, the inert material is colloidal gold, silver, or platinum.

In some embodiments, the nanoparticle is further conjugated to a deoxyribonuclease inhibitor. In certain embodiments, the deoxyribonuclease inhibitor is a DNase I inhibitor.

In some embodiments, the nanoparticle is further conjugated to a Kupffer cell-targeting moiety. In some embodiments, a Kupffer cell-targeting moiety is an antibody. In some embodiments, a Kupffer cell-targeting antibody is a full-length antibody, an antigen-binding fragments derived from an antibody (including but not limited to Fab, Fab′, F(ab′)2, Fv), a single chain antibody (scFv), a bispecific antibody, or a tetravalent antibody.

In some embodiments, the nanoparticle has a size distribution from 5 nanometers to 1000 nanometers in diameter.

In various embodiments, the nanoparticles comprise a population of nanoparticles having a size distribution that is homogenous. In other embodiments, the nanoparticles comprise a population of nanoparticles having a size distribution that is heterogenous. In various embodiments, at least 5% of the nanoparticles have approximately the same size diameter, at least 10% of the nanoparticles have approximately the same size diameter, at least 15% of the nanoparticles have approximately the same size diameter, at least 20% of the nanoparticles have approximately the same size diameter, at least 25% of the nanoparticles have approximately the same size diameter, or at least 30% of the nanoparticles have approximately the same size diameter, or at least 30% of the nanoparticles have approximately the same size diameter, or at least 35% of the nanoparticles have approximately the same size diameter, or at least 40% of the nanoparticles have approximately the same size diameter, or at least 45% of the nanoparticles have approximately the same size diameter, or at least 50% of the nanoparticles have approximately the same size diameter, or at least 55% of the nanoparticles have approximately the same size diameter, or at least 60% of the nanoparticles have approximately the same size diameter, or at least 65% of the nanoparticles have approximately the same size diameter, or at least 70% of the nanoparticles have approximately the same size diameter, or at least 75% of the nanoparticles have approximately the same size diameter, or at least 80% of the nanoparticles have approximately the same size diameter, or at least 85% of the nanoparticles have approximately the same size diameter, or at least 80% of the nanoparticles have approximately the same size diameter, or at least 95% of the nanoparticles have approximately the same size diameter, or at least 96% of the nanoparticles have approximately the same size diameter, or at least 97% of the nanoparticles have approximately the same size diameter, or at least 98% of the nanoparticles have approximately the same size diameter, or at least 99% of the nanoparticles have approximately the same size diameter, or up to 100% of the nanoparticles have approximately the same size diameter.

In some embodiments, the nanoparticles have a circulatory half-life of greater than 10 minutes. In other embodiments, the nanoparticles have a circulatory half-life of at least 2 minutes, or at least 3 minutes, or at least 4 minutes, or at least 5 minutes, or at least 6 minutes, or at least 7 minutes, or at least 8 minutes, or at least 9 minutes, or at least 10 minutes, or at least 15 minutes, or at least 20 minutes, or at least 25 minutes, or at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes, or at least 95 minutes, or at least 100 minutes, or at least 200 minutes, or at least 300 minutes, or at least 400 minutes, or at least 500 minutes. In some embodiments, the nanoparticles have a circulatory half-life of not more than about 50, 150, 200, 250, 300, 350, 400, 450, 500, 550, 600, 650, 700, 750, 800, 850, 900, 950, 1000, 1250, 1500, 1750, 2000, 2250, 2500, 2750, 3000, 3250, 3500, 2750, 4000, 4250, 4500, 4750, or 5000 minutes.

In some embodiments, the nanoparticle has a zeta potential from −70 mV to +70 mV. In some embodiments, the nanoparticle has a zeta potential from −60 mV to +60 mV. In various embodiments, the zeta potential is from about −200 mV to about +200 mV, or from about −200 mV to about +175 mV, or about −200 mV to about +150 mV, or about −200 mV to about +100 mV, or about −200 mV to about +75 mV, or about −200 mV to about +50 mV, or about −200 mV to about +25 mV, or about −200 mV to about +0 mV, or from about −175 mV to about +200 mV, or about −150 mV to about +200 mV, or about −125 mV to about +200 mV, or about −100 mV to about +200 mV, or about −75 mV to about +200 mV, or about −50 mV to about +200 mV, or about −25 mV to about +200 mV, or about 0 my to about +200 mV.

Nanoparticles may be produced by any method known in the art, such as ultrasonification and solvent emulsification.

In some embodiments, a nanoparticle can be functionalized by joining it with one or more additional chemical moieties. A nanoparticle may be functionalized by either encapsulating or being conjugated to an additional chemical moiety. A nanoparticle may be conjugated to an additional chemical moiety through either a covalent linkage or a non-covalent linkage. Examples of additional chemical moieties include nucleic acids, proteins, peptides, small molecules, and any other species known in the art to be suitable for encapsulation or conjugation to nanoparticles. In some embodiments, the nanoparticle contains an aqueous core that can efficiently encapsulate hydrophilic moieties, e.g., nucleic acids, proteins, peptides, or small molecules. In some embodiments, the additional chemical moiety is an inhibitory compound. In some embodiments, an inhibitory compound permanently or reversibly inhibits the function of an enzyme. In some embodiments, a chemical moiety targets the nanoparticle to a specific cell type. In some embodiments, a chemical moiety is an inert material that does not readily undergo chemical transformations in a biological setting. Examples of inert materials include colloidal gold, silver, platinum, carbon nanostructures such as or derived from fullerene, nanotubes, and graphene, iron oxide, silicon dioxide, and any other inert material known in the art to be suitable for encapsulation by or conjugation to nanoparticles. Nanoparticles can be functionalized by either encapsulating or being conjugated to a suitable moiety by any method that is known in the art.

The nanoparticle contemplated herein may be functionalized by any method known in the art. See U.S. Pat. Nos. 9,649,391, 9,770,709, Mout, Rubul et al. (2012) “Surface functionalization of nanoparticles for nanomedicine.” Chem Soc Rev vol. 41(7): 2539-44, and Pelaz, Beatriz et al. (2017) “Diverse Applications of Nanomedicine.”ACS Nano 11(3): 2313-2381, which are hereby incorporated by reference.

In some embodiments, the nanoparticles may comprise a detectable label. The label may be an element located in the core of the nanoparticle or a ligand. The label may be detectable because of an intrinsic property of that element of the nanoparticle or by being linked, conjugated or associated with a further moiety that is detectable. Preferred examples of labels include a label which is a fluorescent group, a radionuclide, a magnetic label or a dye. Fluorescent groups include fluorescein, rhodamine or tetramethyl rhodamine, Texas-Red, Cy3, Cy5, etc., and may be detected by excitation of the fluorescent label and detection of the emitted light using Raman scattering spectroscopy (Y. C. Cao, R. Jin, C. A. Mirkin, Science 2002, 297: 1536-1539).

In various other embodiments, the nanoparticles may comprise a radionuclide for use in detecting the nanoparticle using the radioactivity emitted by the radionuclide, e.g., by using PET, SPECT. Examples of radionuclides commonly used in the art that could be readily adapted for use in the present invention include 99mTc, which exists in a variety of oxidation states although the most stable is ³²P; ³⁷Co; ⁵⁹Fe; ⁶⁷Cu which is often used as Cu^{2h} salts. The general use of radionuclides as labels and tracers is well known in the art and could readily be adapted by the skilled person for use in the aspects of the present invention. The radionuclides may be employed most easily by doping the cores of the nanoparticles or including them as labels present as part of ligands immobilized on the nanoparticles.

At various points in the methods of the disclosure, it may be important to detect the nanoparticles. The nanoparticles of the present disclosure can be detected using a number of techniques well known in the art using a label associated with the nanoparticle or by employing a detectable property of the nanoparticles. These methods of detecting nanoparticles can range from detecting the aggregation that results when the nanoparticles bind to another species, e.g., by simple visual inspection or by using light scattering (transmittance of a solution containing the nanoparticles), to using sophisticated techniques such as transmission electron microscopy (TEM) or atomic force microscopy (AFM) to visualize the nanoparticles. A further method of detecting metal particles is to employ plasmon resonance that is the excitation of electrons at the surface of a metal, usually caused by optical radiation. In some embodiments, the nanoparticles may also comprise a targeting moiety which facilitates the localization and/or association of the nanoparticles at their intended site of action, including targeting moieties that target the Kupffer cells of the liver, target the liver in general, or target DNases in the blood. Such target moieties, and methods for coupling them to nanoparticles, will be well-known in the art. Such targeting moieties may enhance the boosting effect of the nanoparticles through their increase localization to DNases in the blood and Kupffer cells in the liver.

Nanoparticle Formulations

The nanoparticles of the disclosure may be formulated as pharmaceutical compositions that may be in any suitable form, e.g., liquid compositions, solid compositions, gel compositions. Other nanoparticle compositions are also embraced, including that may delivered by transdermal patches, or inhaled as aerosols, dry powder aerosols, emulsions, foams, granules, implants, pellets, pills, sprays, suppositories, suspensions, tablets, and the like, so long as the nanoparticles may be delivered and operate to boost the cfDNA levels in a biological tissue or biological liquid. Such compositions will generally comprise a carrier of some sort, for example a solid carrier or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt. % of the compound. Such formulations are well known in the art.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g., in physiological saline, a dispersion prepared with glycerol, liquid polyethylene glycol, or oils. The nanoparticle compositions can comprise one or more of a pharmaceutically acceptable excipients, carriers, buffers, stabilizers, isotonicising agents, preservatives or anti-oxidants, or other materials well known to those skilled in the art. Such materials should be non-toxic and should not interfere with the efficacy of the active ingredient. The precise nature of the carrier or other material may depend on the route of administration, e.g., intravenously, orally or parenterally.

Liquid pharmaceutical compositions comprising nanoparticle are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

The nanoparticle compositions may also include preservatives. Preservatives are generally included in pharmaceutical compositions to retard microbial growth, extending the shelf life of the compositions and allowing multiple use packaging. Examples of preservatives include phenol, meta-cresol, benzyl alcohol, parahydroxybenzoic acid and its esters, methyl paraben, propyl paraben, benzalconium chloride and benzethonium chloride. Preservatives are typically employed in the range of about 0.1 to 1.0% (w/v).

Preferably, the nanoparticle compositions are given to an individual in a therapeutically effective amount, this being sufficient to show benefit to the individual (i.e., the boosting of cfDNA levels in a biological fluid). Examples of the techniques and protocols mentioned above can be found in Handbook of Pharmaceutical Additives, 2nd Edition (eds. M. Ash and I. Ash), 2001 (Synapse Information Resources, Inc., Endicott, New York, USA); Remington's Pharmaceutical Sciences, 20th Edition, 2000, pub. Lippincott, Williams & Wilkins; and Handbook of Pharmaceutical Excipients, 2nd edition, 1994. By way of example, and the compositions are preferably administered to patients in dosages of between about 0.01 and 100 mg of nanoparticles per kg of body weight, and more preferably between about 0.5 and 1 mg/kg of body weight.

Use of Nanoparticles to Boost cfDNA

In one aspect, the present disclosure relates to the administration to a subject of a therapeutically effective amount of a nanoparticle that results in an increased concentration of cell-free DNA (cfDNA) in one or more biological fluids of the subject.

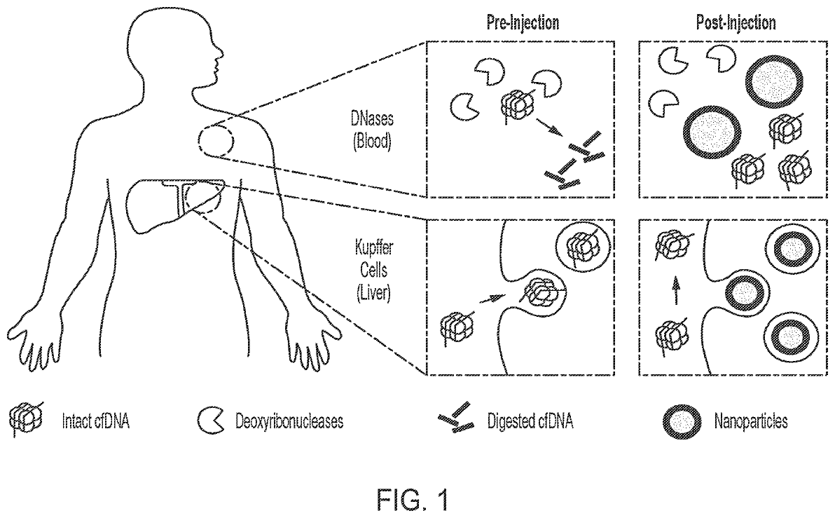

Without desiring to be bound by theory, nanoparticles administered to a subject may transiently increase the concentration of cfDNA found in the biological fluids of a subject, particularly blood, through two distinct mechanisms (FIG. 1). First, nanoparticles that are administered to the bloodstream of a subject (e.g., through intravenous injection) travel to the liver where they are taken up by Kupffer cells. Kupffer cells are liver-resident macrophages that constitutively phagocytose micro- and nanoscale materials found in the blood. Although Kupffer cells utilize phagocytosis to absorb and destroy potentially pathogenic foreign entities circulating in the blood (e.g., bacteria), this is also a primary route by which cellular debris is cleared. Cellular debris consists of any part of a cell that is left over after it dies or is damaged and includes cfDNA. Kupffer cells readily absorb nanoparticles as if they were cellular debris until they reach a point of saturation where they become incapable of effectively phagocytosing and degrading cfDNA. As a result, cfDNA accumulates at a substantially higher concentration in the bloodstream, as compared to when nanoparticles are not administered. Second, nanoparticles administered to the bloodstream of a subject reduce the activity of deoxyribonucleases (DNases) found in the blood. Typically, these extracellular DNases act upon cfDNA, degrading it into progressively shorter fragments. Administered nanoparticles however limit DNase activity in the blood, thereby keeping cfDNA intact. Together, these mechanisms increase the concentration of cfDNA in one or more biological fluids of the subject.

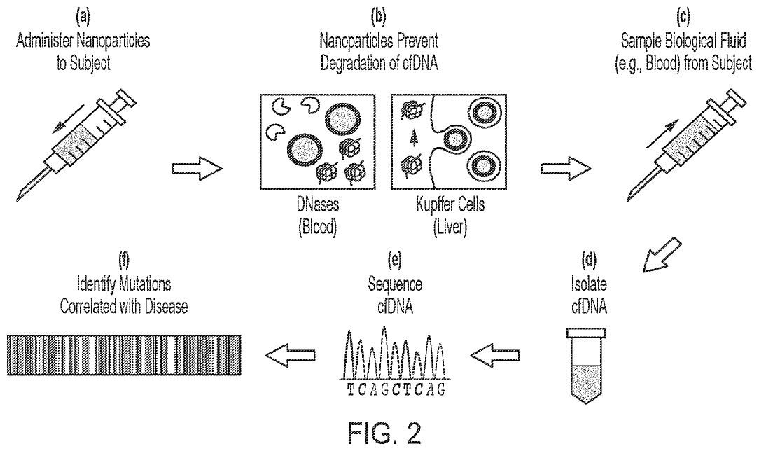

FIG. 2 also provides a schematic indicating one configuration in which nanoparticles enhance the amount of cfDNA recovered by a liquid biopsy (e.g., a blood sample) for use in downstream applications such as sequencing. By recovering an increased amount of cfDNA, sequencing more readily detects mutations in the subject which are indicative of disease. In step (a), the nanoparticles are administered in an effective amount to a subject. The disclosure contemplates the use of any suitable nanoparticles as described herein, formulated in any suitable way, and administered by any suitable means (e.g., by injection). In one embodiment, the nanoparticles are administered in an effective amount to the blood. The dosing can be in accordance with any suitable dosing scheme, as described herein. In addition, the administering of the nanoparticles can be performed as a single dose or multiple doses over a period of time. Step (b) represents the effect of the nanoparticles on the body. In particular, the nanoparticles prevent the degradation of cfDNA. Without being bound by theory, the nanoparticles prevent or otherwise reduce the DNase-dependent degradation of cfDNA in the blood. In addition, the nanoparticles prevent or otherwise reduce the degradation or otherwise removal of cfDNA by the Kupffer cells in the liver. Thus, step (b) represents a period of time whereby, due to the effects of the nanoparticles, the levels of cfDNA become boosted in the blood or other biological fluid. This “boosting” step can be allowed to progress for any suitable length of time, for instance, on the order of minutes to on the order of hours, days, or weeks. Next, once the levels of cfDNA are boosted in the biological fluid (e.g., blood), the biological fluid may be sampled, e.g., by withdrawing a small volume of blood using a needle, as shown in step (c). The sample can then be processed by any known means to isolate the cfDNA from the sample (e.g., any commercially available cfDNA isolation kit), as shown in step (d). The cfDNA is then sequenced in step (e) using any known means for sequencing (e.g., next-generation sequencing (NGS)) to obtained sequence data. The sequence data is then analyzed (e.g., computationally) to identify clinically relevant information, such as mutation information which correlate, predict, or otherwise which are associated with the prediction of a disease condition, or the risk of developing a disease condition. The steps of FIG. 2 are not intended to limit other embodiments of the disclosure.

In some embodiments, administration of a therapeutically effective amount of a nanoparticle results in a concentration of cfDNA that is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, or more than 1000-fold relative to the concentration of cfDNA before the nanoparticle is administered.

In some embodiments, a nominal quantity of nanoparticles is administered to the subject. In some embodiments, between 0.1 quadrillion and 10 quadrillion nanoparticles are administered to the subject. In some embodiments, the number of nanoparticles administered to the subject is determined based on the body weight of the subject. In some embodiments, 0.02 quadrillion nanoparticles are administered per kilogram body weight of the subject.

In some embodiments, the therapeutically effective amount of administered nanoparticles is characterized by the degree to which it limits biological processes that normally result in the removal of cfDNA from one or more biological fluids. In some embodiments, the effective amount of administered nanoparticles inhibits the activity of deoxyribonucleases in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% relative to the activity of the deoxyribonucleases in the absence of the nanoparticle. In some embodiments, the effective amount of administered nanoparticles prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% relative to the uptake of cfDNA by Kupffer cells in the absence of the nanoparticle.

In some embodiments, the administered nanoparticle comprises naturally occurring lipids, synthetic lipids, naturally occurring polymers, or synthetic polymers, or a combination thereof.

In some embodiments, the administered nanoparticle further encapsulates one or more inert materials. In some embodiments, the administered nanoparticle is further conjugated to one or more inert materials. In some embodiments, the inert material is colloidal gold, silver, or platinum.

In some embodiments, the administered nanoparticle further encapsulates a functional moiety. In some embodiments, the administered nanoparticle is further conjugated to a functional moiety. In some embodiments, the functional moiety is a deoxyribonuclease inhibitor. In some embodiments, the inhibitor is a DNase I inhibitor. In some embodiments, the functional moiety is a moiety that targets the administered nanoparticle to Kupffer cells.

In some embodiments, the administered nanoparticle has a size distribution between 5 nanometers and 1000 nanometers in diameter.

In some embodiments, the administered nanoparticle circulates in the bloodstream of the subject with a circulatory half-life of at least 10 minutes. In some embodiments, the administered nanoparticle circulates in the bloodstream of the subject with a circulatory half-life of up to 1 hour, up to 2 hours, up to 3 hours, up to 4 hours, up to 5 hours, up to 6 hours, up to 7 hours, up to 8 hours, up to 9 hours, up to 12 hours, up to 24 hours, up to 36 hours, up to 48 hours, up to 72 hours, or more than 72 hours.

In some embodiments, the administered nanoparticle has a zeta potential from −70 mV to +70 mV. In some embodiments, the administered nanoparticle has a zeta potential from −60 mV to +60 mV.

In some embodiments, the nanoparticle is administered intravenously. In some embodiments, the nanoparticle is administered as an intravenous bolus or infusion.

In some embodiments, the subject is a human patient. In some embodiments, the subject is a human patient that has, is suspected of having, or is at risk of having a disease associated with the presence of cfDNA in one or more biological fluids. In some embodiments, the subject is a human patient that has, is suspected of having, or is at risk of having cancer.

The nanoparticles and compositions of the disclosure may be administered to patients by any number of different routes, including enteral or parenteral routes. Enteral administration includes administration by the following routes: oral, sublingual, and rectal routes. Parenteral administration includes administration by the following routes: intravenous, cutaneous or subcutaneous, nasal, intramuscular, intraocular, transepithelial, intraperitoneal, topical (including dermal, ocular, rectal, nasal, inhalation and aerosol (i.e., pulmonary), and rectal systemic routes.

Administration can be performed e.g., by injection, or ballistically using a delivery gun to accelerate their transdermal passage through the outer layer of the epidermis. The nanoparticles may also be delivered in aerosols. This is made possible by the small size of the nanoparticles.

A nanoparticle may be co-administered with one or more other agents disclosed by this disclosure, such as, but not limited to, an agent capable of binding to cfDNA, and agent capable of inhibiting one or more deoxyribonucleases, or another nanoparticle.

Use of Agents to Bind cfDNA

In another aspect, the present disclosure relates to the administration to a subject of a therapeutically effective amount of one or more agents capable of binding cell-free DNA (cfDNA) that result in an increased concentration of cfDNA in one or more biological fluids of the subject.

Without desiring to be bound by theory, agents capable of binding cfDNA administered to a subject may transiently increase the concentration of cfDNA found in the biological fluids of a subject, particularly blood or urine, by interfering with any combination of endogenous processes for the removal of cfDNA (FIG. 1). First, agents capable of binding cfDNA that are administered to the bloodstream of a subject bind to cfDNA in the subject's blood. By binding to an agent, uptake of cfDNA by Kupffer cells through phagocytosis may be reduced. Accordingly, levels of cfDNA in the bloodstream are transiently increased. Second, binding to an agent may reduce the susceptibility of cfDNA to deoxyribonucleases (DNases) found in one or more biological fluids, such as blood. Extracellular DNases normally act upon cfDNA, degrading it into progressively shorter fragments; however, binding of cfDNA from an agent may protect it from degradation. By either or both of these mechanisms, agents that bind cfDNA may increase the concentration of cfDNA in one or more biological fluids of the subject. Separately, an agent capable of binding cfDNA may increase renal clearance of cfDNA from the bloodstream through the glomerulus membrane of the kidneys, thereby increasing the level of cfDNA that may be collected in urine. By any combination of these mechanisms, an agent capable of binding cfDNA may enhance the amount of cfDNA subsequently recovered by a liquid biopsy (e.g., a blood sample or a urine sample) for use in downstream applications such as sequencing.

In some embodiments, administration of a therapeutically effective amount of an agent capable of binding cfDNA results in a concentration of cfDNA that is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, or more than 1000-fold relative to the concentration of cfDNA before the agent capable of binding cfDNA is administered.

In some embodiments, the effective amount of administered agent capable of binding cfDNA is characterized by the degree to which it limits biological processes that normally result in the removal of cfDNA from one or more biological fluids. In some embodiments, the effective amount of administered agent capable of binding cfDNA reduces the activity of deoxyribonucleases upon cfDNA in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% relative to the activity of the deoxyribonucleases in the absence of the agent. In some embodiments, the effective amount of administered agent capable of binding cfDNA prevent uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% relative to the uptake of cfDNA by Kupffer cells in the absence of the agent.

Ideally, an agent capable of binding cfDNA to be administered to a subject is small, such that it is able to travel through the bloodstream and infiltrate tumors, as well as being non-toxic and non-immunogenic. In some embodiments, the administered agent capable of binding cfDNA is a protein. In some embodiments, the administered agent capable of binding cfDNA is an antibody specific for double stranded DNA (dsDNA). In some embodiments, an antibody that is specific for dsDNA binds to dsDNA irrespective of the dsDNA sequence. In some embodiments, an antibody that is specific for dsDNA is a single domain antibody, also referred to as a nanobody. In some embodiments, the administered agent capable of binding cfDNA is a protein that is not an antibody.

In some embodiments, the administered agent capable of binding cfDNA is of a relatively small size, such that it is capable of binding cfDNA in the bloodstream and then traveling into the urine. In some embodiments, an agent capable of binding cfDNA is 50 kDa in size or smaller, or is more preferably 30 kDa in size or smaller. In some embodiments, an agent capable of binding cfDNA is 20 kDa in size or smaller. In some embodiments, an agent capable of binding cfDNA is 15 kDa in size or smaller. In some embodiments, an agent capable of binding cfDNA is 10 kDa in size or smaller. In some embodiments, an agent capable of binding cfDNA is 5 kDa in size or smaller. In some embodiments, the agent capable of binding cfDNA is a protein that is 20 kDa in size or smaller, 15 kDa in size or smaller, 10 kDa in size or smaller, or 5 kDa in size or smaller.

In some embodiments, the administered agent may only partially prevent degradation of cfDNA by deoxyribonucleases located in a biological fluid (e.g., blood). In such embodiments, cfDNA that is bound by the administered agent is partially protected from the activity of DNases, such that the ends of the cfDNA molecule are degraded, but the region of the cfDNA molecule that is in contact with the agent are protected. In such embodiments, a sufficiently long cfDNA molecule may be preserved (e.g., approximately 50 base pairs in length), such that it may be collected from one or more biological fluids, sequenced, and analyzed (e.g., mapped to a particular region of the subject's genome).

In some embodiments, the administered agent capable of binding cfDNA comprises protamine. In humans, protamine is encoded by either of two genes, PRM1 and PRM2, the product of which are also referred to as P1 and P2. Endogenous protamine is highly cationic and binds to DNA for packaging during spermatogenesis. Use of administered protamine to reverse the anticoagulant effect of heparin during heparin overdose, uncontrolled blood loss, or following cardiovascular surgical procedures is well known in the art (see, e.g., Boer C, et al. “Anticoagulant and side-effects of protamine in cardiac surgery: a narrative review.” Br J Anaesth. 2018 May; 120(5):914-927, which is incorporated by reference herein). Protamine is typically administered to humans as a stable salt of protamine sulphate.

The amino acid sequence of human (Homo sapiens) protamine (PRM1) is as follows (NCBI Reference Sequence: NP_002752.1):

(SEQ ID NO: 1)
MARYRCCRSQSRSRYYRQRQRSRRRRRRSCQTRRRAMRCCRPRYRPRCR
RH.

Human (H. sapiens) protamine (PRM1) is encoded by the following genetic sequence (NCBI Reference Sequence: NM_002761.3):

(SEQ ID NO: 2)
ACAGCCCACAGAGTTCCACCTGCTCACAGGTTGGCTGGCTCAGCCAAGG
TGGTGCCCTGCTCTGAGCATTCAGGCCAAGCCCATCCTGCACCATGGCC
AGGTACAGATGCTGTCGCAGCCAGAGCCGGAGCAGATATTACCGCCAGA
GACAAAGAAGTCGCAGACGAAGGAGGCGGAGCTGCCAGACACGGAGGAG
AGCCATGAGGTGCTGCCGCCCCAGGTACAGACCGCGATGTAGAAGACAC
TAATTGCACAAAATAGCACATCCACCAAACTCCTGCCTGAGAATGTTAC
CAGACTTCAAGATCCTCTTGCCACATCTTGAAAATGCCACCATCCAATA
AAAATCAGGAGCCTGCTAAGGAACAATGCCGCCTGTCAATAAATGTTGA
AAAGTCA.

In some embodiments, the administered agent is a functional variant of protamine, such as, for example, a functional variant of protamine that is at least 70%, at least 80%, at least 90%, at least 95%, or at least 99% identical to the sequence of human (Homo sapiens) protamine (PRM1) provided in SEQ ID NO: 1.

In some embodiments, the administered agent capable of binding cfDNA comprises a DNA-binding protein 7d (Sso7d) homolog, such as that encoded by certain Crenarchaeota species. Sso7d is also referred to as 7 kDa DNA-binding protein d. In some embodiments, the administered agent capable of binding cfDNA is a recombinantly expressed Sso7d from Sulfolobus solfataricus (also known as Saccharolobus solfataricus and Saccharolobus caldissimus). In some embodiments, the administered agent capable of binding cfDNA is a recombinantly expressed Sso7d from another Crenarcheota species, such as Sulfolobus acidocaldarius. Endogenous Sso7d acts as a hyperthermostable histone-like protein in Crenarcheota and is thought to perform several functions related to maintaining archaeal genomes at high temperature, such as stimulating cleavage of Holliday junctions (see, e.g., Guagliardi A, et al., “The Sso7d protein of Sulfolobus solfataricus: in vitro relationship among different activities.” Archaea (Vancouver, B.C.) vol. 1,2 (2002): 87-93; and Kalichuk V, et al. “The archaeal ‘7 kDa DNA-binding’ proteins: extended characterization of an old gifted family.” Scientific Reports (2016) 6, 37274, which is incorporated by reference herein).

The amino acid sequence of S. solfataricus Sso7d is as follows (UniProtKB/Swiss-Prot: P39476.2):

(SEQ ID NO: 3)
MATVKFKYKGEEKEVDISKIKKVWRVGKMISFTYDEGGGKTGRGAVSEK
DAPKELLQMLEKQKK.

S. solfataricus Sso7d is encoded by the following genetic sequence (Gene ID: 44128276):

(SEQ ID NO: 4)
ATGGCAACAGTAAAGTTCAAGTACAAGGGAGAAGAGAAGGAAGTAGATA
TAAGTAAGATAAAGAAGGTATGGAGAGTAGGCAAAATGATAAGTTTCAC
CTATGATGAGGGTGGAGGAAAGACTGGTAGAGGAGCTGTAAGCGAGAAA
GACGCTCCAAAAGAACTACTACAAATGTTAGAAAAGCAAAAGAAATAA.

In some embodiments, the administered agent is a functional variant of Sso7d, such as, for example, a functional variant of Sso7d that is at least 70%, at least 80%, at least 90%, at least 95%, or at least 99% identical to the sequence of S. solfataricus Sso7d provided in SEQ ID NO: 3.

In some embodiments, the administered agent capable of binding cfDNA comprises a DNA binding protein or DNA binding protein domain that does not comprise protamine or Sso7d. In some embodiments, the administered agent capable of binding cfDNA comprises a DNA-binding protein or a DNA-binding protein domain thereof that is specific for double stranded DNA (dsDNA), such as, but not limited to, a catalytically inactive Cas9 endonuclease (i.e., dCas9, e.g., a Staphylococcus aureus dCas9 (UniProtKB: A0A3861RG9)), a catalytically inactive restriction enzyme (e.g., a catalytically inactive type I restriction enzyme (EC 3.1.21.3), a catalytically inactive type II restriction enzyme (EC 3.1.21.4), or a catalytically inactive type III restriction enzyme (EC 3.1.21.5)), a catalytically inactive zinc finger nuclease (ZFN), a zinc finger domain (ZFD), a catalytically inactive transcription activator-like effector nuclease (TALEN), a transcription activator-like effector (TALE), a transcription factor (e.g., heat shock factor (HSF), c-Myc, nuclear factor 1 (NF1)), a leucine zipper domain, a high mobility group (HMG)-box domain, a protein specific for methylated DNA (e.g., a catalytically inactive Escherichia coli Mrr restriction system protein), or an antibody or antibody fragment that is specific for dsDNA. In some embodiments, an agent specific for dsDNA is specific for a general feature of dsDNA, such as the major groove or minor groove of dsDNA. In some embodiments, an agent specific for dsDNA is specific feature of dsDNA, such as a specific nucleotide sequence or a specific nucleic acid modification pattern, such as DNA methylation (e.g., conversion of cytosine to 5-methylcytosine). In some embodiments, an agent specific for dsDNA is a DNA-binding domain of a DNA-binding protein. In some embodiments, an agent specific for dsDNA is a protein or protein domain that is homologous to a known DNA-binding protein or DNA-binding protein domain. In some embodiments, an agent specific for dsDNA is a protein or protein domain that comprises an amino acid sequence that is at least 70%, at least 75%, at least 80%, at least 85%, at least 90%, at least 95%, or at least 99% identical to the amino acid sequence of a known DNA-binding protein or DNA-binding protein domain.

In some embodiments, the administered agent capable of binding cfDNA comprises a non-protein polymer that is specific for dsDNA, such as, but not limited to, a polyamide, such as those comprising, N-methylimidazole (Im), N-methylpyrrole (Py), and N-methyl-3-hydroxypyrrole (Hp), which bind to the minor groove of dsDNA between specific DNA nucleobase pairs. Principles for designing a polyamide to specifically bind to a desired sequence are generally known in the art, such as, for example, in Hidaka T, and Sugiyama H. “Chemical Approaches to the Development of Artificial Transcription Factors Based on Pyrrole-Imidazole Polyamides.” Chem Rec. 2021 June; 21(6):1374-1384, Blackledge MS, and Melander C. “Programmable DNA-binding small molecules.” Bioorg Med Chem. 2013 Oct. 15; 21(20):6101-14; and Dervan P B. “Molecular recognition of DNA by small molecules.” Bioorg Med Chem. 2001 September; 9(9):2215-35, each of which are incorporated by reference herein.

In some embodiments, the administered agent capable of binding cfDNA comprises an antibody that is specific for dsDNA. In some embodiments, the administered agent capable of binding cfDNA comprises an immunoglobulin G (IgG) comprising a heavy chain amino acid sequence and a light chain amino acid sequence that is specific for dsDNA. In some embodiments, an IgG specific for dsDNA is a subtype 1 IgG (IgG1) or subtype 2 IgG (IgG2). Examples of monoclonal antibodies specific for dsDNA and means for their production are well known in the art, such as, for instance, disclosed in Sun K H, et al. “Monoclonal anti-double-stranded DNA autoantibody stimulates the expression and release of IL-1beta, IL-6, IL-8, IL-10 and TNF-alpha from normal human mononuclear cells involving in the lupus pathogenesis.” Immunology vol. 99.3 (2000): 352-60; Hsieh S C, et al. “Monoclonal anti-double stranded DNA antibody is a leucocyte-binding protein to up-regulate interleukin-8 gene expression and elicit apoptosis of normal human polymorphonuclear neutrophils.” Rheumatology (Oxford). 2001 August; 40(8):851-8; Kubota T, et al. “A monoclonal anti-double stranded DNA antibody from an autoimmune MRL/Mp-lpr/lpr mouse: specificity and idiotype in serum immunoglobulins.” Immunol Lett. 1986 Nov. 17; 14(1):53-8; Jang Y J, at al. “Heavy chain dominance in the binding of DNA by a lupus mouse monoclonal autoantibody.” Mol Immunol. 1996 February; 33(2):197-210; Wellmann U, et al. “The evolution of human anti-double-stranded DNA autoantibodies.” Proc Natl Acad Sci USA. 2005 Jun. 28; 102(26):9258-63; Radic M Z, et al. “Structural patterns in anti-DNA antibodies from MRL/lpr mice.” Cold Spring Harb Symp Quant Biol. 1989; 54 Pt 2:933-46; Behar S M and Scharff M D. “Somatic diversification of the S107 (T15) VH11 germ-line gene that encodes the heavy-chain variable region of antibodies to double-stranded DNA in (NZB×NZW) F1 mice.” Proc Natl Acad Sci USA. 1988 June; 85(11):3970-4; Smith R G and Voss E W Jr. “Variable region primary structures of monoclonal anti-DNA autoantibodies from NZB/NZW F1 mice.” Mol Immunol. 1990 May; 27(5):463-70; and Shlomchik M J, et al. “Structure and function of anti-DNA autoantibodies derived from a single autoimmune mouse.” Proc Natl Acad Sci USA. 1987 December; 84(24):9150-4, each of which are incorporated by reference herein. Various monoclonal antibodies specific for dsDNA are commercially available (e.g., Abcam #ab27156; Creative Diagnostics #DMAB-Z7789; Creative Diagnostics #DMABT-Z60446; Millipore Sigma #MAB1293).

In some embodiments, an antibody capable of binding dsDNA is an IgG2 specific for dsDNA, such as, for example, monoclonal antibody clone 3519, which is commercially available from several sources (e.g., Abcam #ab27156, Santa Cruz Biotechnology #sc-58749, Creative Biolabs #MOR-A1358-YJ) and comprises the following heavy chain and light chain amino acid sequences:

Anti-dsDNA monoclonal antibody 35I9 heavy chain:
(SEQ ID NO: 5)
QVQLQQSEAELARPGASVKMSCKASGYTFTRYWMQWVKQRPGQALDWIG
AIYPGNSNTNYNQKFKDKAKLTAVTSASTAYMELSSLTSEDSAVYYCAR
RHYYANNYAMDYWGQGTSVTVSSAKTTAPSVYPLAPVCGDTTGSSVTLG
CLVKGYFPEPVTLTWNSGSLSSGVHTFPAVLQSDLYTLSSSVTVTSSTW
PSQSITCNVAHPASSTKVDKKIEPRGPTIKPCPPCKCPAPNLLGGPSVF
IFPPKIKDVLMISLSPMVTCVVVDVSEDDPDVQISWFVNNVEVLTAQTQ
THREDYNSTLRVVSALPIQHQDWMSGKEFKCKVNNKDLPAPIERTISKP
KGSVRAPQVYVLPPPEEEMTKKQVTLTCMVTDFMPEDIYVEWTNNGKTE
LNYKNTEPVLDSDGSYFMYSKLRVEKKNWVERNSYSCSVVHEGLHNHHT
TKSFSRTPGK.
Anti-dsDNA monoclonal antibody 35I9 light chain:
(SEQ ID NO: 6)
QIVLTQSPAIMSASPGEKVTMTCSASSSVSYMHWYQQKSGTSPKRWIYD
TSKLASGVPARFSGSGSGTSYSLTISSMEAEDSATYYCQQWSSNPPTFG
AGTKLELKRADAAPTVSIFPPSSEQLTSGGASVVCFLNNFYPKDINVKW
KIDGSERQNGVLNSWTDQDSKDSTYSMSSTLTLTKDEYERHNSYTCEAT
HKTSTSPIVKSFNRNEC.

In some embodiments, an antibody capable of binding dsDNA is a full-length antibody. In some embodiments, an antibody capable of binding dsDNA is an antibody fragment, such as that generated from the sequence of a known full-length antibody (e.g., an IgG). For example, an antibody fragment specific for dsDNA may comprise an antibody fragment that is in the format of, though not limited to, a F(ab) fragment, a F(ab′) 2 fragment, a ScFv fragment, a ScFv-Fc fragment, or another antibody fragment format that is generally known in the art. An antibody fragment specific for dsDNA may comprise, for example, a F(ab) fragment, such as that described in Stanfield R L and Eilat D. “Crystal structure determination of anti-DNA Fab A52.” Proteins. 2014 August; 82(8):1674-8; Barbas S M, et al. “Human autoantibody recognition of DNA.” Proc Natl Acad Sci USA. 1995 Mar. 28; 92(7):2529-33; and Kowal C, et al. “Molecular mimicry between bacterial and self antigen in a patient with systemic lupus erythematosus.” Eur J Immunol. 1999 June; 29(6):1901-11, each of which are incorporated by reference herein. Alternatively, an antibody fragment specific for dsDNA may comprise, for example, a scFv fragment, such as that described in Kim Y R, et al. “Heavy and light chain variable single domains of an anti-DNA binding antibody hydrolyze both double- and single-stranded DNAs without sequence specificity.” J Biol Chem. 2006 Jun. 2; 281(22):15287-95, which is incorporated by reference herein.

In some embodiments, an antibody capable of binding dsDNA is a single-domain antibody (i.e., a nanobody, VHH, or sdAb), such as that which is generated from the sequence of a known IgG antibody. Means for recombinantly generating single-domain antibodies, including those derived from a known IgG, are readily available in the art (see, e.g., Muyldermans S. “A guide to: generation and design of nanobodies.” FEBS J. 2021 April; 288(7):2084-2102, which is incorporated by reference herein). Methods for producing single-domain antibodies suitable for use in human subjects are also well known in the art (see, e.g., Crowell L E, et al. “Development of a platform process for the production and purification of single-domain antibodies.” Biotechnol Bioeng. 2021 Feb. 24. doi: 10.1002/bit.27724. Epub ahead of print, which is incorporated by reference herein.

In embodiments where an agent capable of binding cfDNA is a protein (e.g., an antibody or another protein) or nucleic acid (e.g., DNA or RNA), the agent may comprise one or more mutations. A mutation is defined as an insertion, a deletion, or a substitution occurring at a specific position in an amino acid or nucleic acid sequence. In some embodiments, the agent comprises one or more mutations relative to a reference sequence, such as a wild-type (“native”) sequence (e.g., a wild-type amino acid sequence). The similarity between two amino acid sequences or two nucleic acid sequences (e.g., the sequence of a protein agent capable of binding cfDNA and the sequence of a wild-type protein) may be characterized by the percent of residues (e.g., amino acids or nucleotides) that are identical between the two sequences (i.e., “% identity”). In some embodiments, an agent capable of binding cfDNA is at least 60% identical, at least 65% identical, at least 70% identical, at least 75% identical, at least 80% identical, at least 85% identical, at least 90% identical, at least 95% identical, or at least 99% identical to a reference sequence, such as a wild-type sequence. In some embodiments, the presence of one or more mutations in the sequence of an agent capable of binding cfDNA enhances the binding affinity between the agent and cfDNA. In some embodiments, the presence of one or more mutations in the sequence of an agent capable of binding cfDNA increases the level of cfDNA in one or more biological fluids of a subject when the agent is administered, relative to when the same agent without the one or more mutations is administered to the subject.

In embodiments where an agent capable of binding cfDNA is an antibody specific for dsDNA, the antibody is a recombinantly produced full-length antibody or antibody fragment thereof (e.g., a single-domain antibody) that has reduced binding to (i.e., decreased affinity for) one or more endogenously expressed receptor proteins which contribute to receptor-mediated clearance of DNA immune complexes, such as, for example fragment crystallizable (Fc) receptors, e.g., Fc gamma (γ) receptor. Such an antibody or antibody fragment that is specific for dsDNA may comprise, for example, one or more amino acid insertions, deletions, or substitutions in its Fc region which reduce binding to Fc γ receptor, such as, but not limited to, L234A/L235A/P329G substitutions, a D265A substitution, or L234A/L235A/N297A substitutions, relative to a Homo sapiens Fc region, such as a H. sapiens IgG Fc region. An amino acid insertion, deletion, or substitution in the Fc region may prevent a posttranslational modification from occurring in the Fc region, such as, for example, an N297A substitution, which prevents N-linked glycosylation of the Fc region at N297. An antibody or antibody fragment specific for dsDNA may alternatively or additionally comprise one or more amino acid substitutions, additions, or deletions which enhance the circulatory half-life of the antibody or antibody fragment by changing its isotype to an antibody isotype with a relatively long circulatory half-life (e.g., IgG4). An antibody or antibody fragment specific for dsDNA may lack an Fc region.

In embodiments where an agent capable of binding cfDNA is an antibody specific for dsDNA, the antibody is a recombinantly produced full-length antibody or antibody fragment thereof (e.g., a single-domain antibody) that has enhanced binding to one or more endogenously expressed receptor proteins which impede receptor-mediated clearance of DNA immune complexes, such as, for example, the neonatal Fc receptor (FcRn). Such an antibody or antibody fragment that is specific for dsDNA may comprise, for example, one or more amino acid insertions, deletions, or substitutions in its Fc region which enhance binding to FcRn, such as, but not limited to, M252Y/S254T/T256E substitutions, or M428L/N434S substitutions, relative to a Homo sapiens Fc region, such as a H. sapiens IgG Fc region.

In some embodiments, the administered agent capable of binding cfDNA circulates in the bloodstream of the subject with a circulatory half-life of at least 10 minutes. In some embodiments, the administered agent capable of binding cfDNA circulates in the bloodstream of the subject with a circulatory half-life of up to 1 hour, up to 2 hours, up to 3 hours, up to 4 hours, up to 5 hours, up to 6 hours, up to 7 hours, up to 8 hours, up to 9 hours, up to 12 hours, up to 24 hours, up to 36 hours, up to 48 hours, up to 72 hours, or more than 72 hours.

In some embodiments, the agent capable of binding cfDNA is administered intravenously. In some embodiments, the agent capable of binding cfDNA is administered as an intravenous bolus or infusion.

In some embodiments, the subject is a human patient. In some embodiments, the subject is a human patient that has, is suspected of having, or is at risk of having a disease associated with the presence of cfDNA in one or more biological fluids. In some embodiments, the subject is a human patient that has, is suspected of having, or is at risk of having cancer.

The agents capable of binding cfDNA and compositions of the disclosure may be administered to patients by any number of different routes, including enteral or parenteral routes. Enteral administration includes administration by the following routes: oral, sublingual, and rectal routes. Parenteral administration includes administration by the following routes: intravenous, cutaneous or subcutaneous, nasal, intramuscular, intraocular, transepithelial, intraperitoneal, topical (including dermal, ocular, rectal, nasal, inhalation and aerosol (i.e., pulmonary)), and rectal systemic routes.

Administration can be performed e.g., by injection, or ballistically using a delivery gun to accelerate their transdermal passage through the outer layer of the epidermis. Agents capable of binding cfDNA may also be delivered in aerosols. This is made possible by the small size of the agents.

An agent capable of binding cfDNA may be co-administered with one or more other agents disclosed by this disclosure, such as, but not limited to, a nanoparticle, an agent capable of inhibiting one or more deoxyribonucleases, or another agent capable of binding cfDNA.

Use of Agents to Inhibit Deoxyribonucleases

In another aspect, the present disclosure relates to the administration to a subject of a therapeutically effective amount of one or more agents capable of inhibiting deoxyribonucleases that result in an increased concentration of cell-free DNA (cfDNA) in one or more biological fluids of the subject.

Without desiring to be bound by theory, agents capable of inhibiting deoxyribonucleases (DNases) when administered to a subject may transiently increase the concentration of cfDNA found in the biological fluids of a subject, particularly blood or urine, by interfering with the activity of DNases located in one or more biological fluids of the subject. In particular, such agents may inhibit the activity of one or more extracellular DNases located in the bloodstream which typically act upon cfDNA, degrading it into progressively shorter fragments. Accordingly, an agent capable of inhibiting DNases (a DNase inhibitor) may enhance the amount of cfDNA subsequently recovered by a liquid biopsy (e.g., a blood sample or a urine sample) for use in downstream applications such as sequencing.

In some embodiments, administration of a therapeutically effective amount of an agent capable of inhibiting one or more DNases results in a concentration of cfDNA that is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, or more than 1000-fold relative to the concentration of cfDNA before the agent capable of inhibiting one or more DNases is administered.

In some embodiments, the effective amount of administered agent capable of inhibiting one or more DNases is characterized by the degree to which it limits biological processes that normally result in the removal of cfDNA from one or more biological fluids. In some embodiments, the effective amount of administered agent capable of inhibiting one or more DNases the activity of DNases upon cfDNA in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% relative to the activity of the deoxyribonucleases in the absence of the agent.

In some embodiments, an agent capable of inhibiting one or more DNases is an inhibitor of one or more endogenous DNases of the subject. In some embodiments, an agent capable of inhibiting one or more DNases comprises an inhibitor of DNase I, which is encoded by the human gene DNASE1 (also known as DNL1 or DRNI; Gene ID: 1773) and typically found in the bloodstream where it degrades chromatin from apoptotic cells. Agents capable of inhibiting DNaseI and derivatives thereof are well known in the art, as described in Kolarevic A, et al. “Deoxyribonuclease inhibitors.” Eur J Med Chem. 2014 Dec. 17; 88:101-11, which is incorporated herein by reference.

In some embodiments, the administered agent capable of inhibiting one or more DNases circulates in the bloodstream of the subject with a circulatory half-life of at least 10 minutes. In some embodiments, the administered agent capable of inhibiting one or more DNases circulates in the bloodstream of the subject with a circulatory half-life of up to 1 hour, up to 2 hours, up to 3 hours, up to 4 hours, up to 5 hours, up to 6 hours, up to 7 hours, up to 8 hours, up to 9 hours, up to 12 hours, up to 24 hours, up to 36 hours, up to 48 hours, up to 72 hours, or more than 72 hours.

In some embodiments, the agent capable of inhibiting one or more DNases is administered intravenously. In some embodiments, the agent capable of inhibiting one or more DNases is administered as an intravenous bolus or infusion

In some embodiments, the subject is a human patient. In some embodiments, the subject is a human patient that has, is suspected of having, or is at risk of having a disease associated with the presence of cfDNA in one or more biological fluids. In some embodiments, the subject is a human patient that has, is suspected of having, or is at risk of having cancer.

The agents capable of inhibiting one or more DNases and compositions of the disclosure may be administered to patients by any number of different routes, including enteral or parenteral routes. Enteral administration includes administration by the following routes: oral, sublingual, and rectal routes. Parenteral administration includes administration by the following routes: intravenous, cutaneous or subcutaneous, nasal, intramuscular, intraocular, transepithelial, intraperitoneal, topical (including dermal, ocular, rectal, nasal, inhalation and aerosol (i.e., pulmonary)), and rectal systemic routes.

Administration can be performed e.g., by injection, or ballistically using a delivery gun to accelerate their transdermal passage through the outer layer of the epidermis. Agents capable of binding cfDNA may also be delivered in aerosols. This is made possible by the small size of the agents.

An agent capable of inhibiting one or more DNases may be co-administered with one or more other agents disclosed by this disclosure, such as, but not limited to, a nanoparticle, an agent capable of binding to cfDNA, or another agent capable of inhibiting one or more DNases.

cfDNA Assays

In another aspect, the method of administering to a subject a therapeutically effective amount of a nanoparticle, an agent capable of binding to cfDNA, or an agent capable of inhibiting deoxyribonucleases that results in an increased concentration of cell-free DNA (cfDNA) in one or more biological fluids of the subject can be applied to a method of increasing the concentration of cfDNA in one or more biological fluids of a subject and then assaying the cfDNA (FIG. 2). The cfDNA may be assayed by any means known in the art, such as but not limited to spectrophotometry, binding of fluorescent probes, polymerase chain reaction (PCR), real-time PCR, DNA damage assays, DNA repair assays, and DNA sequencing. In some embodiments, cfDNA is isolated from the biological fluid prior to assaying. In some embodiments, cfDNA is assayed in the biological fluid.

Method for Isolating cfDNA

The methods and systems disclosed herein are suitable for increasing the concentration of cfDNA in a biological fluid, including, but not limited to, blood, plasma, and urine. It will be appreciated, however, that cfDNA may be obtained from any suitable biological sample or specimen, such as a biological fluid (e.g., but blood, plasma, blood fractions, urine, saliva, cerebral spinal fluid, cord blood, placental fluid, fluids from cytology, etc.), a tissue (e.g., a tissue obtained from surgery, biopsy, transplant, autopsy, etc., including any bodily tissue, including any epithelium tissue, muscle tissue, connective tissue, or nervous tissue), or an organ sample (e.g., an organ of the digestion system (e.g., esophagus, stomach, liver, pancreas, small intestine, large intestine, rectum, anus), an organ of the respiratory system (e.g., lung, nose, trachea, bronchi), an organ of the excretion system (e.g., kidney, urinary bladder, urethra), an organ of the circulatory system (e.g., heart/cardiac, blood vessels, spleen), or an organ of the nervous system (e.g., brain, spinal cord), or any organ of any of the known organ system of the body, including the integumentary system, skeletal system, muscular system, lymphatic system, respiratory system, digestive system, nervous system, endocrine system, cardiovascular system, urinary system, and reproductive system)).

The biological sample containing cfDNA may also be obtained using any available biopsy technique, including a needle-biopsy technique (including techniques such as fine-needle aspiration, core needle biopsy, vacuum-assisted biopsy, or image-guided biopsy), an endoscopic biopsy, a skin biopsy (e.g., a shave biopsy, punch biopsy, incisional biopsy, or excisional biopsy), a bone marrow biopsy, or a surgical biopsy.

In various embodiments, the methods and systems disclosed herein for increasing the amount of cfDNA may involve the use of a liquid biopsy.

A liquid biopsy is a simple and non-invasive alternative to surgical biopsies which enables doctors to discover a range of information about a tissue (e.g., a tumor) through a simple blood sample. Liquid biopsy is a type of technique for sampling and analyzing of non-solid biological tissues, mainly used in disease diagnosis. Traces of disease-related DNA (e.g., tumor-related DNA) in the blood can give clues about which treatments are most likely to work for that patient. In certain embodiments, liquid biopsies provide an opportunity for detecting, analyzing and monitoring cancer in various body effluents such as blood or urine instead of a fragment of cancer tissue. It is composed of different biological matrices such as circulating tumor cells (CTCs), cell free nucleic acids (e.g., cfDNA), exosomes or tumors. In addition to representing a non- or minimally invasive procedure, liquid biopsy may represent a better view of tumor heterogeneity and allows for real-time monitoring of cancer evolution and treatment. Recent technological and molecular advances, greatly facilitated by the use of microfluidics in many cases, have permitted large progresses both in our ability to purify and analyze liquid biopsy components. The present disclosure may be used with any liquid biopsy approach known in the art, such as those described in the following references, each of which are incorporated in their entireties by reference:
Yadav et al., “Detection of circulating tumour cells in colorectal cancer: Emerging techniques and clinical implications,” World J Clin Oncol. 2021 Dec. 24; 12(12):1169-1181;
Bunduc et al., “Exosomes as prognostic biomarkers in pancreatic ductal adenocarcinoma a systematic review and meta-analysis,” Transl Res. 2022 Jan. 20:S1931-5244;
Takami H, et al., “Advances in Molecular Profiling and Developing Clinical Trials of CNS Germ Cell Tumors: Present and Future Directions,” Curr Oncol Rep. 2022 Jan. 20; and
Li et al., “Liquid biopsy in lung cancer: significance in diagnostics, prediction, and treatment monitoring,” Mol Cancer. 2022 Jan. 20; 21(1):25;
Underwood et al., “Liquid biopsy for cancer: review and implications for the radiologist,” Radiology, Nov. 19, 2019

To assay cfDNA in accordance with the disclosure herein, it may be desirable to first isolate the cfDNA from a biological sample, such as blood.

In some embodiments, the biological sample can be freshly collected. In some embodiments, the biological sample can be stored and then used in the methods and compositions described herein. In some embodiments, the sample is an untreated sample. As used herein, “untreated sample” means a biological sample that has not been previously treated in any way, except for dilution and/or suspension in solution. In some embodiments, a biological sample can be utilized in the methods and compositions described herein after it is obtained from a subject and stored or processed. As a non-limiting example, the sample can be embedded, refrigerated, or frozen in paraffin wax. After thawing, a frozen sample can be assessed for the presence of nucleic acid according to the methods and compositions described herein. In some embodiments, the sample can be a processed or unprocessed sample. Exemplary methods for processing a biological sample include centrifugation, filtration, sonication, homogenization, heating, freeze-thawing, contact with a preservative (e.g., an anticoagulant or nuclease inhibitor), and the like. Arbitrary combinations may be mentioned, but not limited to these. In some embodiments, the biological sample can be treated with chemical and/or biological reagents. Chemical and/or biological reagents can be used to protect and/or maintain the stability of the sample or the nucleic acid contained in the sample (e.g., cfDNA) during processing and/or storage. In addition, or alternatively, chemical and/or biological reagents can be used to release nucleic acids (e.g., cfDNA) from other components of the sample (e.g., from protein aggregates). As a non-limiting example, a blood sample can be used in the methods and compositions described herein after being treated with an anticoagulant. Those skilled in the art are well aware of methods and processes for processing and storing samples for nucleic acid (e.g., cfDNA) analysis. In some embodiments, the sample can be a liquid sample that has been clarified, e.g., by centrifugation. In some embodiments, the sample can be clarified by low speed centrifugation (e.g., 3,000×g or less) and the supernatant containing a clear liquid sample can be collected. In some embodiments, the cfDNA can first be amplified by PCR to amplify the material to be analyzed. The isolated cfDNA and/or amplification product thereof can be isolated from the enzyme, primer, or buffer components before sequencing or further analysis. Methods for isolating nucleic acids are well known in the art.

In some embodiments, the one or more biological fluids includes blood. In some embodiments, the one or more biological fluids include a processed or partially processed fluid that is obtained from a biological fluid initially obtained from the subject, such as serum or plasma.

In some embodiments, the cfDNA is assayed at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours after the therapeutically effective amount of nanoparticles is administered to the subject. In some embodiments, the cfDNA is assayed not longer than 25 hours after the therapeutically effective amount of nanoparticles is administered to the subject.

In some embodiments, assaying the cfDNA includes sequencing (e.g., next-generation sequencing methods), real-time PCR, digital droplet PCR, and other known PCR and sequencing methods of analyzing DNA. In some embodiments, sequencing the cfDNA further includes identifying the presence of mutations in the cfDNA which are indicative of a disease. In some embodiments, the subject is identified as having a disease when sequencing of cfDNA results in the detection of mutations that are indicative of the disease.

In some embodiments, the subject is a human patient who has, is suspected of having, or is at risk for a disease (e.g., cancer) associated with the presence of cfDNA in one or more biological fluids. In some embodiments, the subject is a human patient who has, is suspected of having, or is at risk for particular disease-causing mutations. In some embodiments, the disease is a cancer. In some embodiments, the cancer may include, though is not limited to, colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma.

Accordingly, in some embodiments a method of treatment for cancer is provided which comprises administering to a human patient having, suspected of having, or at risk for cancer an effective amount of nanoparticles sufficient for increasing the concentration of cfDNA in one or more biological fluids of the patient, optionally collecting a sample of biological fluid from the patient (e.g. a blood biopsy), optionally isolating cfDNA from the sample, sequencing the cfDNA, identifying mutations in the cfDNA that are associated with cancer, and administering to the patient an one or more therapies for treatment of the cancer. In some embodiments, therapies are selected from an administration of an effective amount of a chemotherapeutic compound, an effective amount of an immunotherapeutic compound, and/or an effective amount of a hormone therapeutic compound to kill cancer cells or limit their proliferation. In some embodiments, a targeted therapy is selected that is intended to specifically kill or limit the proliferation of cancer cells by interfering with one or more biological process required by those cells for survival. In some embodiments, therapies are selected from radiation therapy, radiofrequency ablation, cryoablation, and/or surgical intervention to remove cancerous or pre-cancerous tissue. In some embodiments, selected therapies include transplantation of healthy donor cells and/or organs to the patient, such as to replace diseased cells and/or organs.

In some embodiments, a method of screening for cancer is provided which comprises administering to a human subject who has previously had or is at risk for cancer an effective amount of nanoparticles sufficient for increasing the concentration of cfDNA in one or more biological fluids of the patient, optionally collecting a sample of biological fluid from the patient (e.g. a blood biopsy), optionally isolating cfDNA from the sample, sequencing the cfDNA, and identifying mutations in the cfDNA that are associated with cancer. In some embodiments, a subject who has previously had or is at risk for cancer is found to have mutations associated with cancer, wherein the method may further comprise administering to the patient one or more therapies for treatment of the cancer.

Sequencing of cfDNA

In various embodiments, assaying the cfDNA can involve obtaining the sequence of the cfDNA or a portion thereof. This can include obtaining the sequence of an amplified product of the cfDNA.

In some embodiments, the isolated cfDNA can be sequenced by next generation sequencing methods. In some embodiments, the next generation sequencing method comprises a method selected from the group consisting of Ion Torrent, Illumina, SOLiD, 454; Massively Parallel Signature Sequencing, solid phase reversible dye terminator sequencing; and DNA nanoball sequencing. As used herein, “next generation sequencing” refers to the speeds that were not possible with conventional sequencing methods (e.g., Sanger sequencing) by reading thousands of millions of sequencing reactions simultaneously.

Next generation sequencing techniques and sequencing primer designs are well known in the art (e.g., Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp 133-141; Su, et al., “Next-generation sequencing and its applications in molecular diagnostics,” Expert Rev Mol Diagn, 2011, 11 (3): 333-43; Zhang et al., “The impact of next-generation sequencing on genomics,” J Genet Genomics, 2011, 38 (3): 95-109; (Nyren, P. et al. Anal Biochem 208: 17175 (1993): Bentley, DR Curr Opin Genet Dev 16: 545-52 (2006); Strausberg, R L, et al. Drug Disc Today 13: 569-77 (2008); U.S. Pat. Nos. 7,282,337; 7,279,563; 7,226,720; 7,220,549; 7,169,560; see and No. 20070070349); U.S. Pat. Nos. 6,818,395; 6,911,345; U.S. Patent Application Publication No. 2006/0252077; No. 2007/0070349. The entire contents of these general references on next-generation sequencing are incorporated herein by reference.

cfDNA Amplification

Analysis of the isolated cfDNA boosted by the methods and composition described herein may involve an amplification step. Amplification may be carried out by PCR. PCR requires the use of a nucleic acid polymerase. As used herein, the phrase “nucleic acid polymerase” refers to an enzyme that catalyzes the template-dependent polymerization of nucleoside triphosphates to form a primer extension product that is complementary to the template nucleic acid sequence. The nucleic acid polymerase enzyme begins synthesis at the 3 ‘ end of the annealed primer and proceeds in the direction toward the 5’ end of the template. A number of nucleic acid polymerases are known in the art and are commercially available. One group of preferred nucleic acid polymerases is thermostable, i.e., even after they have been subjected to a temperature sufficient to denature the annealed strand of complementary nucleic acids, e.g., 94° C. or sometimes higher.

As understood in the art, PCR requires a cycle that includes a strand separation step that typically involves heating the reaction mixture. As used herein, the term “strand separation” or “separate strands” refers to the separation of a complementary double-stranded molecule into two single strands that can be utilized to anneal to an oligonucleotide primer. More particularly, strand separation according to the methods described herein is performed by heating a nucleic acid sample above its T m (i.e., melting temperature). In general, heating to 94° C. is sufficient to perform strand separation on samples containing nucleic acid molecules in a buffer suitable for nucleic acid polymerase. Exemplary buffers include 50 mM KCl, 10 mM Tris-HCl (pH 8.8 at 25° C.), 0.5 to 3 mM MgCl2, and 0.1% BSA.

Similarly, as is understood in the art, PCR requires the step of annealing a primer to a template nucleic acid. Since a template nucleic acid is defined as a single-stranded nucleic acid that a given primer specifically anneals, any strand of a target nucleic acid can be a template nucleic acid. As used herein, “anneal” refers to the step of hybridizing two complementary or substantially complementary nucleic acid strands, and more particularly a template-dependent polymerase when used in the context of PCR. It means the step of hybridizing to form a primer extension substrate for the enzyme. Conditions for primer-target nucleic acid annealing vary with primer length and sequence and are based on the T m calculated for the primer. In general, the annealing step in an amplification regimen involves reducing the temperature after the strand separation step to a temperature based on the T m calculated for the primer sequence for a period sufficient to allow such annealing. T m is a number of any widely available algorithms (e.g., OLIGO™ (Molecular Biology Insights Inc. Colorado) primer design software and VENTRO NTI™ (Invitrogen, Inc. California) primer design software, and Primer3, Including Oligo Calculator, and NetPrimer (available for free on Premier Biosoft; Palo Alto, CA; and the World Wide Web at www.premierbiosoft.com/netprimer/netprlaunch/Help/xnetprlaunch.html).

As will be appreciated, PCR requires the use of amplification primers or oligonucleotides. Methods for obtaining, synthesizing, or otherwise making and designing PCR primers is well known in the art. Primers according to the methods and compositions described herein have a length of less than or equal to 300 nucleotides, such as a length of 300, or 250, or 200, or 150, or 100, or 90, or 80, or 70, or 60, or 50, or less than or equal to 40 nucleotides, or less than or equal 30 nucleotides, or less than or equal 20 nucleotides, or less than or equal to 15 nucleotides, but can be at least 10 nucleotides. Methods for making primers are well known in the art, and numerous companies are able to provide oligonucleotide synthesis services suitable for providing primers according to the methods and compositions described herein, such as INVITROGEN™ Custom DNA oligos; Life Technologies, Grand Island, NY, or IDT custom DNA oligos; Coralville, IA.

Formulations for Increasing the Concentration of cfDNA

In another aspect, the present disclosure provides formulations that may be used to increase the concentration of cfDNA in one or more biological fluids of a subject, comprising one or more agents capable of increasing the concentration of cfDNA in a subject. In some embodiments, such a formulation may contain any of the nanoparticles, agents for binding cfDNA, or agents for inhibiting deoxyribonucleases that are described herein, or any combination of these agents thereof.

In some embodiments, a formulation comprising various agents for increasing the concentration of cfDNA in one or more biological fluids of a subject also comprises one or more of a pharmaceutically acceptable excipients, carriers, buffers, stabilizers, isotonicising agents, preservatives or antioxidants, or other materials well known to those skilled in the art. Such materials should be non-toxic and should not interfere with the efficacy of the active ingredient. The precise nature of the carrier or other material may depend on the route by which the formulation is to be administered to a subject, e.g., intravenously, orally or parenterally.

In various embodiments, the nanoparticles, agents for binding cfDNA, or agents for inhibiting deoxyribonucleases may be formulated together as a single composition.

In other embodiments, the nanoparticles, agents for binding cfDNA, or agents for inhibiting deoxyribonucleases may be formulated as separate compositions.

In still other embodiments, the nanoparticles may be formulated with agents for binding cfDNA or agents for inhibiting deoxyribonucleases.

In other embodiments, agents for binding cfDNA may be formulated with the nanoparticles or agents for inhibiting deoxyribonucleases.

In yet other embodiments, the agents for inhibiting deoxyribonucleases may be formulated together with the nanoparticles or the agents for binding cfDNA.

In certain aspects, one of ordinary skill in the art can deliver one or more of the herein disclosed cfDNA-boosting agents (e.g., the nanoparticles, agents for binding cfDNA, or agents for inhibiting deoxyribonucleases) locally, i.e., proximal to the source of the cfDNA. For example, such local delivery may be achievable by injection or microneedle patch, thereby protecting the cfDNA in the location it is generated, e.g., in a tumor microenvironment. In certain other aspects, one of ordinary skill in the art can specifically target delivery of one or more of the herein disclosed cfDNA-boosting agents (e.g., the nanoparticles, agents for binding cfDNA, or agents for inhibiting deoxyribonucleases) to the bodily location generating the cfDNA, e.g., a tumor microenvironment, a particular cell, tissue, or organ that is producing the cfDNA, by further modifying one or more of the cfDNA-boosting agents (e.g., the nanoparticles, agents for binding cfDNA, or agents for inhibiting deoxyribonucleases) with a targeting moiety which targets the agent to a specific cell, tissue, or bodily location (e.g., to a tumor or tumor microenvironment). Such targeting moieties are well known in the art.

In some embodiments, agents for binding cfDNA, i.e., cfDNA-binding agents may be delivered to a target cell, tissue, or bodily site (e.g., to a tumor site) that is generating cfDNA (e.g., a tumor microenvironment) by using a bispecific antibody strategy. Such a strategy may involve, in some embodiments, a bispecific antibody that is designed comprising two different antigen binding regions, wherein one antigen binding region binds to a tumor target and the other antigen binding region comprising a cfDNA-binding moiety. In this way, the bispecific antibody may be designed such that it targets a tumor cell or tissue of interest, thereby causing the co-delivery of the cfDNA-binding moiety to the tumor or tumor microenvironment, whereby it may bind and protect the cfDNA in the vicinity, thereby providing an increased half-life of the cfDNA of interest. The bispecific antibodies may target any suitable cellular marker of interest (e.g., a tumor marker), which may include, but are not limited to EGFR (cancer marker associated with solid tumors), ANG2 (cancer marker associated with solid tumors), EpCAM (cancer marker associate with malignant ascites), HER2 (breast cancer marker), Trop2 (triple negative breast cancer), Nectin4 (urothelial cancer marker), CEA (gastrointestinal adenocarcinoma marker and solid tumor marker), DLL3 (small cell lung cancer marker), c-MET (non-small cell lung cancer), LGR5 (colorectal cancer marker), PSMA (prostate cancer marker), or EFGRvIII (glioblastoma marker). As contemplated herein, a suitable bispecific antibody would target one of these target-specific antigens (or another target-specific antigen known in the art) to direct the cf-DNA-binding moiety to bind to cfDNA in the vicinity of the tumor microenvironment, and in particular, tumor-derived cfDNA. In other embodiments, the bispecific antibodies could be further modified with disease protease-specific masking moieties which block the binding function of a bispecific antibodies until and unless a disease-specific protease cleaves the masking moiety, thereby activating the bispecific antibody. Such disease-specific proteases may including, but are not limited to cancer-associated enzymes, such as, but not limited to, MMP, kallikreins, cathepsins, plasminogen activator, ADAM (A disintegrin and metalloprotease), MMP-7, ADAMS, ADAM15, matriptase, kallikrein 3, ADAM 15, kallikrein 6, cathepsin B, peptidase IV, and seperase, as well as cardiovascular disease proteases (e.g., angiotensin converting enzyme), atherosclerosis (e.g., cathepsin K, L, S), and arthritis-associated enzymes (MMP-1), as well as protease such as uPA, matriptase, tPA, MMP2, or MMP9.

Kits for Increasing the Concentration of cfDNA

In another aspect, the present disclosure relates to kits for the use of nanoparticles, agents capable of binding to cfDNA, or agents capable of inhibiting deoxyribonucleases to increase the concentration of cfDNA in one or more biological fluids of a subject. In some embodiments, a kit comprises nanoparticles sufficient for increasing the concentration of cfDNA in a subject, an agent capable of binding cfDNA sufficient for increasing the concentration of cfDNA in a subject, and/or an agent capable of inhibiting the activity of deoxyribonucleases sufficient for increasing the concentration of cfDNA in a subject. In some embodiments, a kit disclosed herein further comprises a pharmacologically acceptable fluid in which the nanoparticles are stored, and/or instructions for administration of the kit. Such a kit may be utilized in the course of determining whether a subject has a disease associated with cfDNA in one or more biological fluids. In particular instances, such a kit may be utilized in the course of determining whether a human subject possesses mutations in cfDNA that are indicative of cancer.

In some embodiments, the nanoparticles of the kit are a population of nanoparticles, as described herein, that are sufficient for increasing the concentration of cfDNA in one or more biological fluids when administered to a subject. In some embodiments, the nanoparticles are liposomes. In some embodiments, the nanoparticles comprise one or more lipids, one or more polymers, or a combination thereof. In some embodiments, the nanoparticles are further functionalized by encapsulating or conjugation to an additional moiety. In some embodiments, nanoparticles may be characterized by their shape, size, size distribution, dispersity, circulatory half-life, and zeta potential, as described herein. In some embodiments, nanoparticles can be characterized as being relatively homogenous or heterogeneous with respect to size, shape, and/or composition.

In some embodiments, the agents capable of binding to cfDNA of the kit are sufficient for increasing the concentration of cfDNA in one or more biological fluids when administered to a subject. In some embodiments, the agents capable of binding to cfDNA comprise one or more proteins. In some embodiments, the agents capable of binding to cfDNA are characterized according to their size. In some embodiments, the agents capable of binding to cfDNA are of a sufficiently small size that they are capable of binding to cfDNA in one biological fluid of a subject and then traveling to another, such as binding cfDNA in the blood of a subject and then traveling into the urine. In some embodiments, the agents capable of binding to cfDNA comprise protamine, a Sso7d homolog, or an antibody specific for double stranded DNA (dsDNA). In some embodiments, the protamine is in the form of protamine sulphate. In some embodiments, the Sso7d homolog is Sulfolobus solfataricus Sso7d. In some embodiments the antibody specific for dsDNA is a monoclonal antibody, such as a single domain antibody.

In some embodiments, the nanoparticle kits can comprise one or more of a pharmaceutically acceptable excipients, carriers, buffers, stabilizers, isotonicising agents, preservatives or antioxidants, or other materials well known to those skilled in the art. Such materials should be non-toxic and should not interfere with the efficacy of the active ingredient. The precise nature of the carrier or other material may depend on the route of administration, e.g., intravenously, orally or parenterally.

In some embodiments, the kit is to be stored below 50° C., below 40° C., below 30° C., below 20° C., below 10° C., below 0° C., below −10° C., or below −20° C. such that the nanoparticles are relatively stable over time.

In some embodiments, the nanoparticles of the kit are administered intravenously. In some embodiments, the nanoparticles of the kit and the pharmacologically acceptable fluid in which they are stored are contained within a glass vial or equivalent container that maintains the sterility and stability of the nanoparticles prior to administration. In some embodiments, the nanoparticles are retrieved from the glass vial or equivalent container and intravenously delivered to the subject by means of a syringe fitted with a hypodermic needle, the use of which is well known in the art.

In some embodiments, the kit is in the format of a liquid biopsy kit. In embodiments where the kit is utilized as a liquid biopsy kit, the kit can be used to boost the level of cfDNA in a biological fluid (e.g., blood) and optionally then to isolate and/or analyze the biological fluid from the subject, e.g., to detect one or more mutations in the cfDNA. In some embodiments, collection of a biological sample is conducted at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours, but not longer than 25 hours, after using the kit to boost the level of cfDNA in a biological sample of a subject. In some embodiments, the kit is in the format of a blood biopsy kit, wherein use of the kit to boost cfDNA levels is followed by collection and analysis of blood from the subject. In some embodiments, analysis of cfDNA is conducted directly in the biological fluid (e.g., blood). In some embodiments, analysis of cfDNA is conducted after isolating the cfDNA from the biological fluid (e.g., blood). In some embodiments, analysis of cfDNA involves sequencing of cfDNA and identification of one or more disease-causing mutations present in the cfDNA. In some embodiments, the disease-causing mutations in question are associated with a cancer, such as colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, or soft tissue sarcoma.

### EXAMPLES

### Example 1—Nanoparticles Increase cfDNA Recovered by Blood Biopsies and Enhance Cancer Detection

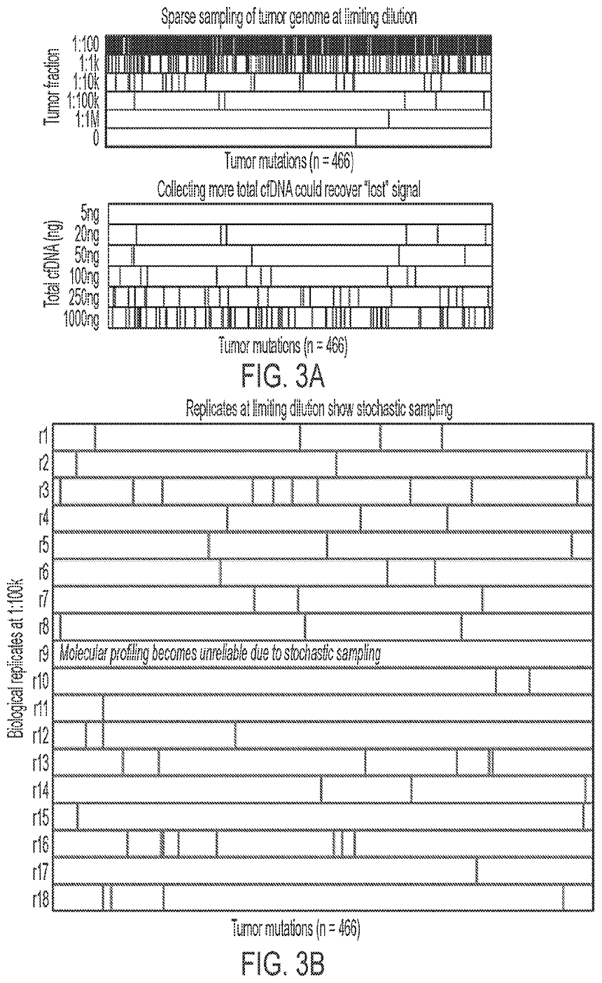

Liquid biopsies have garnered tremendous interest for their ability to detect and profile cancer genomes from the “cell-free” DNA in a liquid sample, such as a blood draw. Major efforts are underway pursuing liquid biopsies for earlier detection of cancer, monitoring of treatment response, detection of minimal residual disease, tracking of cancer evolution, and selection of precision cancer therapy. Yet, a major challenge remains: most individuals have very little cell-free DNA (cfDNA) in their blood and most of it comes from healthy cells. Thus, when the fraction of tumor-derived cfDNA fragments is very low relative to total cfDNA fragments, there will be very sparse sampling of the cancer genome in, for instance, a blood draw (FIG. 3A,B). This is what referred to as “limiting dilution” and it is a major challenge for liquid biopsy testing. While most efforts to date have focused on increasing the analytical sensitivity of liquid biopsies (such as deeply sequencing more of the genome via error correction methods), no amount of sequencing can overcome physical absence of tumor DNA.

A method to increase the total amount of cfDNA sampled without appreciably changing its tumor fraction could shift the balance from “limiting dilution” to more complete representation of the cancer genome in each blood draw, as has been established in 1:100k dilutions (FIG. 3C). This could have a profound impact on everything from enabling the detection of clinically actionable mutations from patients with inoperable tumors or whose tumors may have evolved over time; to detecting minimal residual disease after cancer therapy and determining which patients need further treatment; to ultimately enhancing the sensitivity of early cancer detection tests. In this sense, the disclosed method sits ‘upstream’ of most blood biopsy tests and stands to directly improve their performance, much like how contrast agents are used to enhance imaging scans.

Here a nanoparticle has been developed for cfDNA recovery that can quantitatively boost the overall quantity of cfDNA in circulation. This is the first time that a nanoparticle that increases the input molecules recovered for analysis from a blood biopsy has been described. This nanoparticle could increase the sensitivity of cfDNA, overcoming the primary limitation of this analyte.

The nanoparticle consists of lipid nanoparticles (liposomes) that are delivered about 1-hour prior to blood sampling and function by increasing the circulation time of cfDNA in blood. This nanoparticle could be directly perturbing two of the fundamental driving forces in cfDNA clearance—Kupffer cells (KCs) and extracellular nucleases. KCs in the liver uptake and degrade cfDNA including nucleosomes (biological nanoparticles of DNA wrapped around histone proteins)³. On the other hand, extracellular nucleases degrade cfDNA in circulation⁴. Liposomes may be transiently saturating uptake routes in KCs, and hence attenuating cfDNA clearance from the circulation. In parallel, liposomes may be protecting cfDNA from degradation by circulating DNases through interaction with nucleosomes or Dnases, hindering cfDNA degradation. As such, tuning the nanoparticle by modulating nanoparticle size, concentration and/or composition to i) increase their ability to saturate KCs and/or ii) further protect cfDNA from degradation by nucleases may further enhance cfDNA recovery.

Results

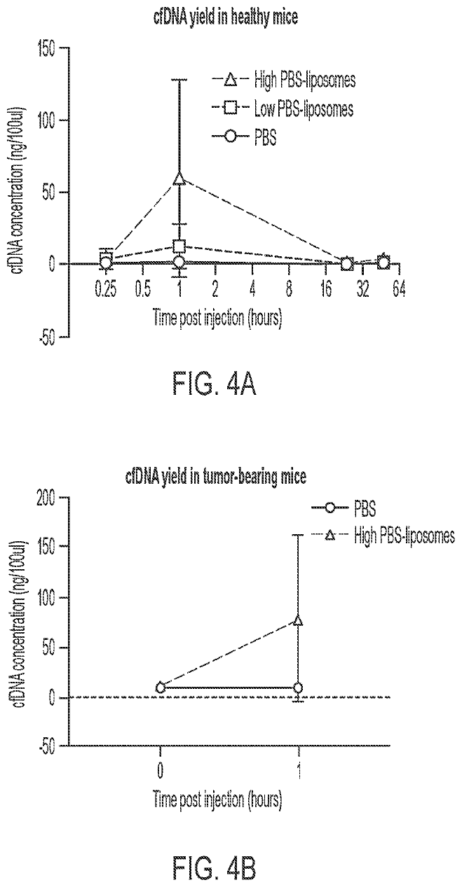

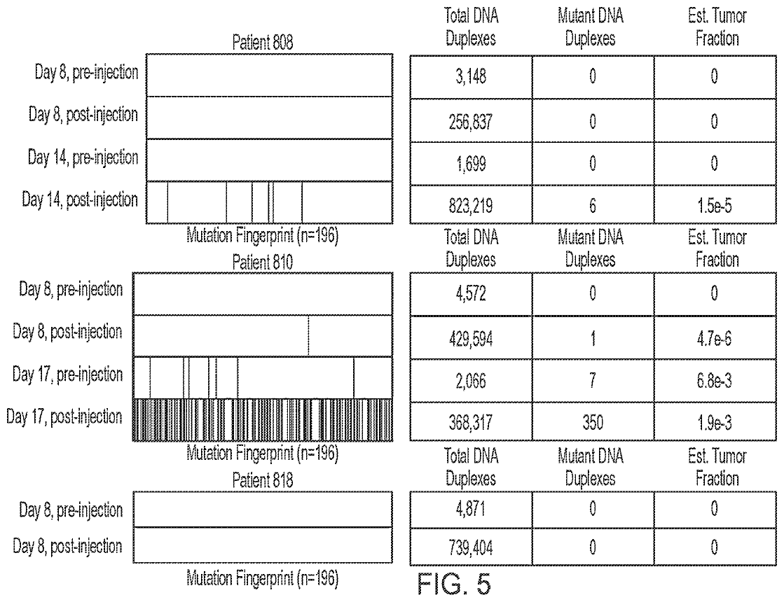

Liposomes at two concentrations were delivered to healthy BALB/c mice and total cfDNA yield from plasma was tracked over time. The liposome injection led to a dose-dependent spike in total cfDNA yield 1-hour post injection (FIG. 4A). Subsequently, treatment with a high liposome dose was tested on tumor-bearing mice (colorectal cancer flank-tumor model). Consistent with previous findings, the liposome injection led to an increase in overall cfDNA yield 1-hour post injection (6-fold on average) in tumor-bearing mice as well (FIG. 4B). By testing for 200 genome-wide tumor mutations that were known to be present in the cancer cell line used for tumor establishment, it was confirmed that substantially more DNA molecules could be recovered after injection compared with before, without appreciably changing tumor fraction (FIG. 5). Accordingly, with more tumor DNA in the sample, a much stronger genome-wide tumor mutation fingerprint could be detected at 1-hour post-injection compared to before (e.g., 350 mutant duplexes versus 7). These data indicate that more of the cancer genome can be recovered using the disclosed nanoparticle.

This proof-of-principle sets the foundation for the development of a cfDNA priming agent, which could have profound implications for cancer management; from early detection to effective therapy selection, treatment response monitoring and disease recurrence prevention, providing invaluable insights at critical junctures in patient care. In addition, as liquid and blood biopsies grow in the clinical area, this approach to boost cfDNA could improve the performance of other existing analytes and may help resolve situations for which a standard blood draw yields borderline or inconclusive results.

### References for Example 1

[1] Bettegowda, C. et al., Sci. Transl. Med 2014, 6: 224ra24
[2] Heitzer, E. et al., Nat Rev Genet. 2019, 20(2):71-88
[3] Gauthier, V. J. et al., J. Immunol. 1996, 156(3):1151-6
[4] Vancevska, A. et al., Laboratory Medicine 2013, 44(2), 125-128

### Example 2—Contrast Agent for Blood Biopsy

Blood biopsies could enable early detection and molecular profiling of cancer but, like imaging scans, have limited signal-to-noise (s/n) ratio and signal intensity in most patients. The s/n ratio informs the detection modality but the signal intensity determines whether the test stands to be useful at all. For instance, blood biopsies have shown the greatest promise for patients with Stage IV cancer whose tumors shed ample cell-free DNA (cfDNA) into the bloodstream—enough to reconstruct the cancer genome from a blood draw¹. Yet, blood biopsies have been much less useful for patients with Stage 0-III cancer. Part of the reason is that sequencing remains too expensive and inaccurate to apply when tumor fraction (or s/n ratio) is low. However, the more fundamental limitation is that most patients shed little tumor cfDNA into blood, which means that signal intensity can be weak or absent in many blood samples—and no amount of sequencing can overcome that.

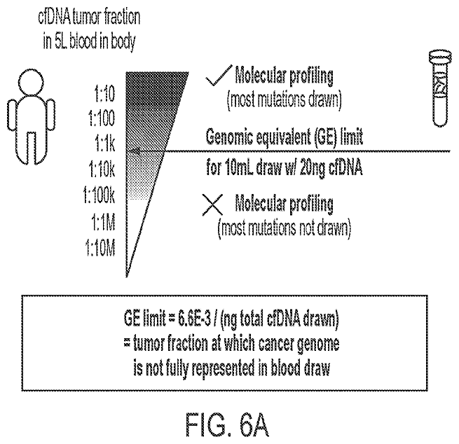

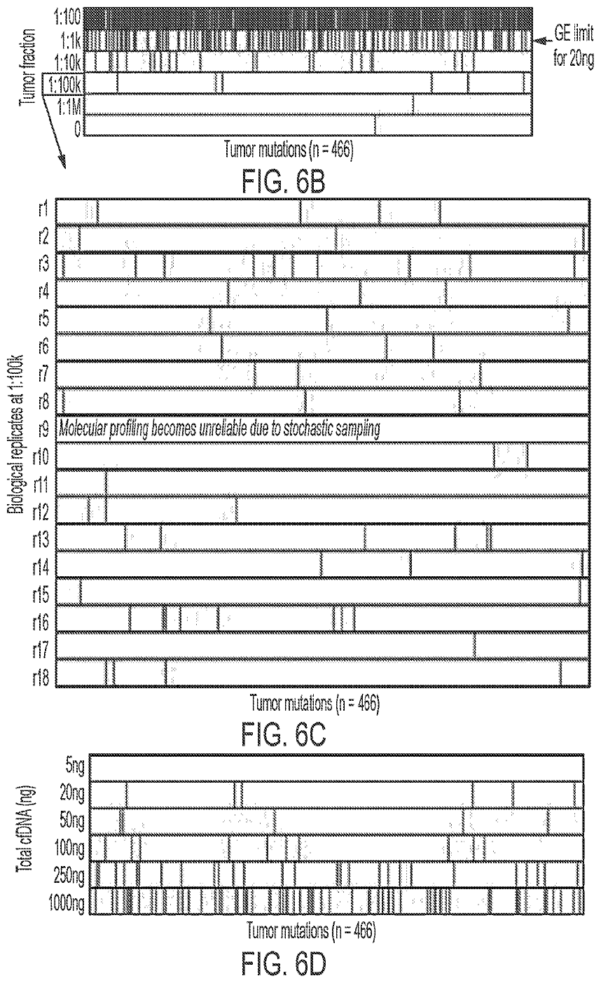

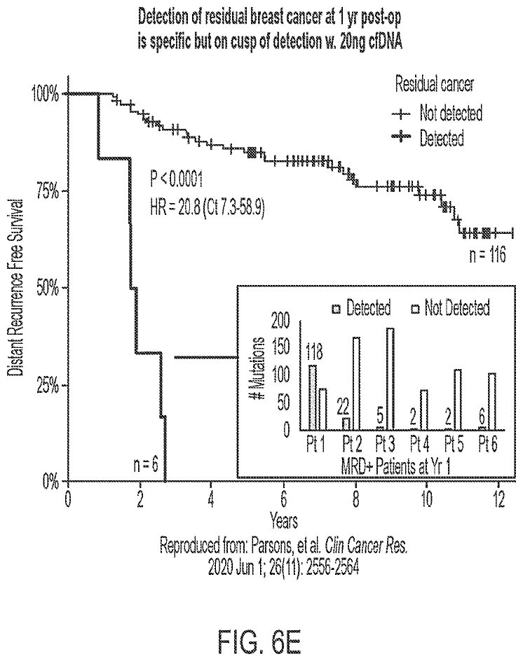

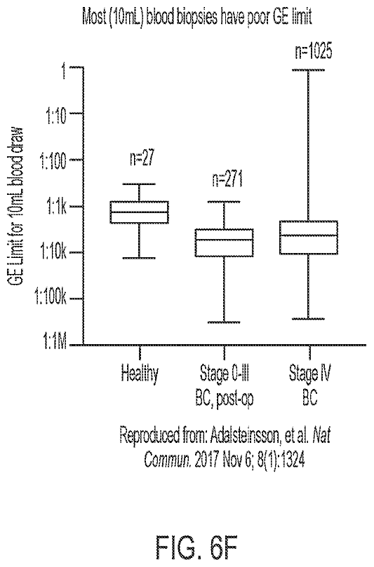

The typical 10 mL blood draw contains only 20 ng of cfDNA, or a few thousand haploid genome equivalents, assuming 6.6 pg per haploid genome. When the tumor fraction of cfDNA in the bloodstream is less than the inverse of this number—termed the genomic equivalent (GE) limit—there will be incomplete representation of the cancer genome in a blood draw (FIG. 6A). Results have confirmed this (FIGS. 6B-6C), and it has serious implications for both cancer detection and molecular profiling. For cancer detection, it means that large swaths of the genome need to be scanned with sufficient accuracy so as to rely on any subset to detect cancer. For molecular profiling, it means that it will not be possible to discern whether mutations were not detected because they were not present in the tumor, or because they were not sampled in the blood draw (FIG. 6C). These challenges manifest for the many patients whose cfDNA tumor fractions are 0.1% or lower, with only ˜20 ng of cfDNA in their blood draws.

The GE limit can be improved by increasing the mass of cfDNA sampled. For instance, the tumor signal that is lost in a 20 ng cfDNA sample at 1:100k tumor fraction can be partially restored by increasing the total cfDNA sampled (FIG. 6D). Yet, that's typically the amount of cfDNA in 500 mL of blood, and it's not safe to draw more than that at once. By tracking many individualized mutations, the most can be made out of the little cfDNA (˜20 ng) in a blood draw. It was found that minimal residual disease (MRD) could be detected after curative intent treatment in breast cancer patients who later experienced metastatic recurrence (FIG. 6E). However, sensitivity at one year post-surgery was limited, and samples which tested positive for MRD were on the cusp of detection: in several, just 2-6 of >100 mutations tracked were detected (FIG. 6E, inset). It was also found that GE limits (based on mass of cfDNA per 10 mL blood) in 1,323 blood samples to be in a similar range of 1/1k-1/8k (FIG. 6F). Such values are likely insufficient for cancer detection and molecular profiling in most patients, which is why signal enhancement is sought.

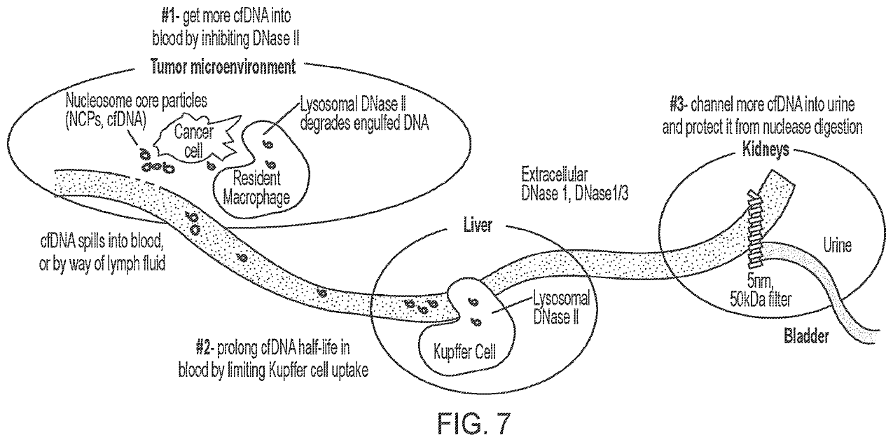

Sampling of cfDNA could be dramatically improved: fifty billion cells die in the human body each day, but only one million cells' worth of cfDNA is found in the 5 L of blood, and the typical 10 mL blood draw samples just 0.2% of that. Exact mechanisms remain elusive 3 but likely involve (1) disequilibrium between rates of cell turnover and efferocytosis when cells are rapidly dividing, (2) a combination of intra- and extracellular nucleases, and (3) Kupffer cells in the liver (FIG. 7). The first two may affect how much cfDNA makes it into the bloodstream, with the intracellular nuclease DNase II eliminating most DNA taken up by macrophages, and spatial confinement likely plays a role too. Meanwhile, Kupffer cells are thought to be a major driving force in the elimination of cfDNA from blood. For instance, studies have shown that 71.0-84.7% of injected nucleosomes (i.e., cfDNA particles) are cleared by Kupffer cells in the liver within 10 min 4, whereas knockout of extracellular nucleases has had little impact on cfDNA yield s, and most cfDNA is too large for renal excretion 6. Some cfDNA is found in urine though, where nucleases become a driving force in cfDNA clearance, having 100-fold greater activity than in plasma.

Interestingly, these challenges have been addressed through different lenses. For instance, Kupffer cells present a major obstacle in nanotherapeutics, leading to poor half-life of nanoparticles, and transient depletion with liposomal clodronate 8 and saturation with empty liposomes 9 have improved half-life in circulation. Nanoparticles have even been engineered to hide from Kupffer cells 10. Yet, nobody has explored the effects on cancer detection of increasing cfDNA yield. Additionally, while the focus in nanomedicine has been on how to get more material into tumors, similar concepts may inform how to get more out from tumors. Previous developments have included protease-cleavable nanosensors, which involve recovering peptides generated deep within tumors, in the blood and then urine¹¹. Meanwhile, previous studies have looked at autoimmune conditions resulting from defective nucleases, and the role of DNA as an antigen. Importantly, it is believed that nucleases such as DNase II can be transiently inhibited in a safe and effective manner, without provoking autoimmunity, and have ongoing efforts to develop a DNase II inhibitor to boost antitumor immune responses. Additionally, previous studies have developed ways to overcome poor s/n ratio of blood biopsies, which refocuses the challenge on how to improve molecular recovery of cfDNA, such that most tumor mutations are sampled in a blood draw. Additionally, the advanced capabilities for antibody engineering which could be used to bind and protect cfDNA from nuclease attack.

Presented herein is the development of a cfDNA contrast agent, to boost the quantitative recovery of tumor-derived cfDNA. Three major driving forces in cfDNA clearance are explored in this Example—the intracellular nuclease DNase II, Kupffer cells, and extracellular nucleases—to mobilize more cfDNA into the bloodstream, prolong its half-life in circulation, and channel more into the urine for concentration and collection (FIG. 7). The impact of each on cancer detection in tumor-bearing mice is also explored herein.

DNase II knockout draws more tumor-derived cfDNA into circulation. Yield and tumor fraction of cfDNA were quantified in blood and urine, before and after inducible knockout of DNase II, in tumor-bearing mice. Without being bound by theory, DNase II knockout mobilizes more cfDNA into circulation because tissue resident macrophages are unable to degrade engulfed DNA.

cfDNA uptake by Kupffer cells prolongs half-life in circulation. Liposomal clodronate was used to deplete Kupffer cells in tumor-bearing mice and quantify the yield and tumor fraction of cfDNA in blood and urine, before and after treatment. The tumor fraction of cfDNA remained constant but the total yield increased, leading to greater absolute recovery of tumor cfDNA. Next, less invasive ways to limit cfDNA uptake by Kupffer cells, without depleting them, were pursued. An in vitro system was developed to study cfDNA uptake and test specific methods for attenuation, such as use of ssDNA, dsDNA, liposomes and polystyrene beads.

Stabilization of cfDNA channeled into urine. A divalent nanobody against dsDNA capable of (i) displacing nucleosome-bound cfDNA, (ii) protecting it from nuclease digestion within a 30-50 bp window, and (iii) channeling more cfDNA into urine and protecting it from further degradation in urine was developed. The yield and size distribution of cfDNA resulting from exposure to the endogenous nucleases was determined in fresh plasma and urine from healthy donors, in the presence or absence of anti-dsDNA nanobodies. Candidates for future in vivo testing were nominated in healthy and tumor-bearing mice.

Most cfDNA is degraded before it reaches the bloodstream, and that which does is rapidly cleared. This has serious implications for detecting low levels of cancer from body via a blood biopsy. By garnering a deeper understanding of the driving forces of cfDNA clearance, and exploring how to modulate them, tumor signal in cfDNA can be boosted (FIG. 6E) and blood biopsies can be made feasible for more patients. The development of a cfDNA contrast agent, could have a profound impact on diagnosing cancer at an early stage, determining whether treatment has worked, and obtaining real-time molecular profiles at critical junctures in patient care. Creating a contrast agent counters the ‘non-invasive’ appeal of blood biopsies but may be necessary (as it has been for medical imaging, FIG. 6A) to enable cancer detection and molecular profiling at critical junctures in care. For instance, millions of individuals have indeterminate nodules on imaging scans each year which could lead to invasive biopsy procedures; millions of patients remain uncertain as to whether or not they have been cured by surgery and are over- or under-treated as a result; surgery could potentially be averted for patients who exhibit complete response to neoadjuvant therapy but that's not currently feasible to assess from blood; and millions are given cancer therapy without a real-time molecular profile of their tumor. In addition, as blood biopsy testing grows in the clinical arena, this approach to boost cfDNA yield could improve the performance of most existing tests, and may help to resolve situations for which a standard blood draw yields a borderline or inconclusive result.

DNase II knockout draws more tumor-derived cfDNA into circulation.

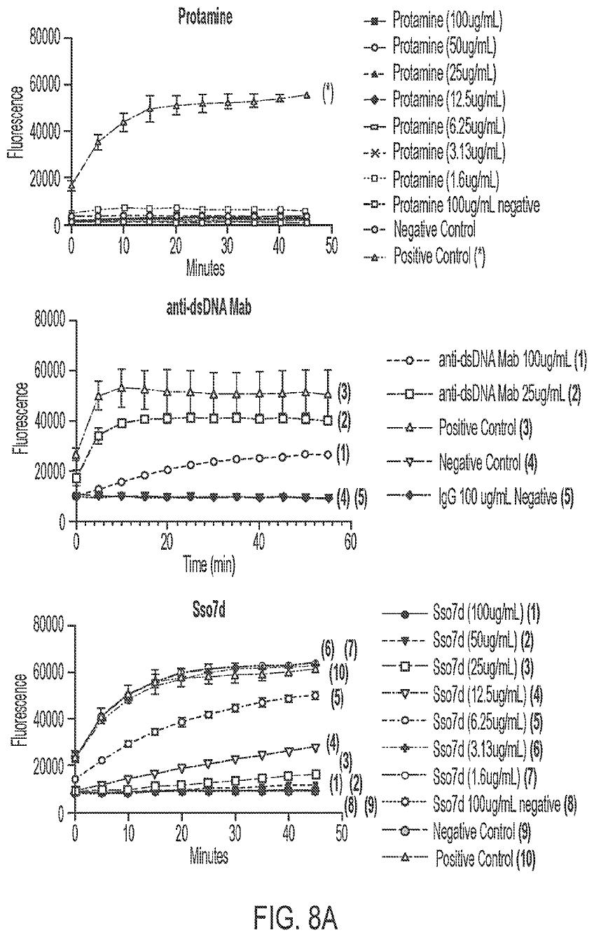

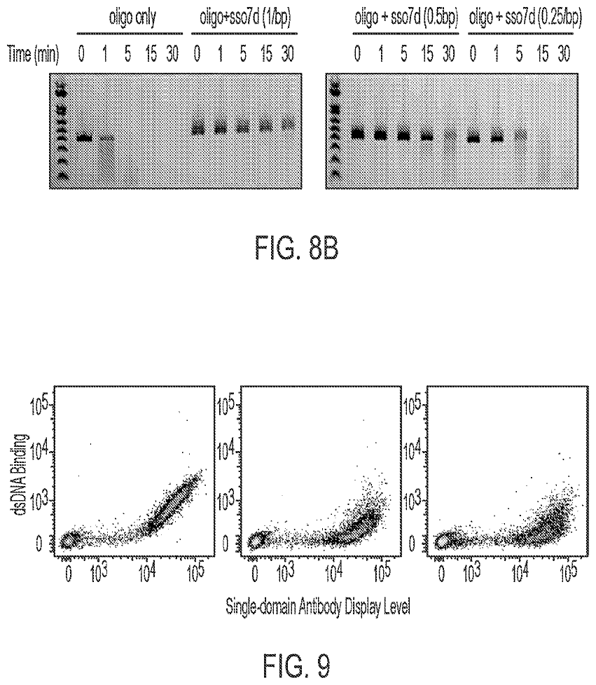

The first major barrier to cancer detection is that most DNA is degraded before it reaches the bloodstream. DNase II is the nuclease responsible for most DNA degradation in the body: it functions in the lysosomes of phagocytes such as macrophages which clear dead cells and debris¹⁴. It also serves to break down DNA which is exported from the nucleus in the context of DNA damage, as commonly occurs when cancer cells replicate¹⁵. It is hypothesized that knocking out DNase II will inhibit DNA clearance in the tumor microenvironment, leading to greater release of cfDNA into lymph and blood vessels. It may also impair the ability of Kupffer cells to clear cfDNA from circulation. Indeed, previous work has shown that 4-6-week-old DNase II^{-/-} IFN-IR^{-/-} mice harbor 10 ug/mL serum¹⁶, which is 1000-fold higher than is typically observed in mice (FIGS. 8A-8B). However, the impact of DNase II knockout on the yield and tumor fraction of cfDNA in tumor-bearing mice has not yet been evaluated. Presented herein is the systemic knockout of DNase II to improve cancer detection.

Experimental approach. cfDNA was collected before and after induction of DNase II deletion in tumor-bearing mice. Using the DnaseII-flox/− model (which uses MX1-Cre to induce gene deletion), 10⁶ B16 melanoma (or MC38 colorectal) cells were subcutaneously transplanted to DnaseII-flox/− mice before induction. When tumors reached 10 mm across (normally by day 9), DNase II knockout was induced by injecting poly-IC intraperitoneally (i.p.), which takes effect in <3 days. Blood (50 ul) and urine were collected every 3 days for 12 days (when tumors in non-induced mice bear tumors >20 mm across to be euthanized) to test sensitivity of detecting cfDNA at different tumor sizes. In addition, control or DNaseII-deficient tumor cells (by CRISPRcas9 editing) were transplanted to wild type mice and sequence and yield of tumor cfDNA was examined. Sensitivity for detecting cfDNA at different tumor sizes (comparing to reference sequence of each tumor) was tested using previously established methods (FIGS. 6C-6E). As each molecule of cfDNA was labeled with a unique DNA barcode, the absolute number of tumor and normal cfDNA fragments in each sample was able to be counted, before and after DNase II knockout. Then, the quantitative yield of tumor cfDNA was correlated with the size of the tumor.

cfDNA Uptake by Kupffer Cells Prolongs Half-Life in Circulation.

The second major barrier to cancer detection is that most cfDNA which makes it into the bloodstream is rapidly eliminated by Kupffer cells in the liver^{4,13}. Studies have shown that Kupffer cells can be transiently depleted for several days and that this increases the circulatory half-life of injected nucleosomes in mice. Yet, nobody has explored how this affects cancer detection in tumor-bearing mice. It is hypothesized that depleting Kupffer cells will improve cancer detection by enabling integrated sampling of more total cfDNA over longer time scales (FIG. 6E). This was not expected to change the tumor fraction in cfDNA, unless resident macrophages in other tissues are depleted too. More cfDNA was expected in urine though, as more accumulates in the bloodstream. Initial efforts focused on Kupffer cell depletion, and subsequently less invasive ways to limit cfDNA uptake by Kupffer cells were explored. Specifically, primary and immortalized macrophage cell lines were used to develop these further as systems to study cfDNA uptake and test ways to attenuate it.

Experimental approach. The findings of Tavares et al⁸, that dosing 0.05 mg of liposomal clodronate per gram weight achieves depletion of Kupffer cells two days after administration in Balb/c mice, were confirmed. Then, the CT26 murine colorectal cancer cell line was used to initiate tumors. This tumor model has been previously shown to shed sufficient cfDNA to enable detection of late stage tumors in mice¹⁷. In addition, the complete genome of the CT26 cell line has been mapped¹⁸, which enabled the tracking of a large fingerprint of mutations specific to this cancer model, using the cfDNA analysis method described above. Yield and tumor fraction of cfDNA in blood and urine were evaluated, before and after Kupffer cell depletion, in tumor-bearing mice. Healthy mice as well as empty liposomes-treated mice were used as control experiments. An in vitro system to study the attenuation of cfDNA uptake and clearance by macrophages was also developed. Previous work developed an in vitro co-culture platform that consists of primary hepatocytes and macrophages and recapitulates the liver microenvironment. This platform was leveraged to screen a library of compounds including ssDNA, dsDNA, liposomes and polystyrene beads, to investigate cfDNA uptake saturation mechanisms. For said screens, the Kupffer cells were treated with the saturating agent and subsequently the cultures were incubated with fluorescently labelled chromatin to measure its uptake. Successful saturating agents that abolish cfDNA uptake by Kupffer cells were then tested in vivo in mice. This in vitro platform can be tailored to murine or human cell lines, making it relevant to both in vivo mice experiments and to clinical translation.

Stabilization of cfDNA channeled into urine. Two major advantages of sampling cfDNA from the urine are (1) it is more accessible than blood, and (2) it provides an integrated sampling of cfDNA from the entire blood volume (e.g., the kidneys produce 180 L of filtrate per day, with 178.5 L of fluid reabsorbed 19). While most cfDNA is too large to pass through the kidneys, a fraction of it can be found in urine: tumor mutations have been found in urine of cancer patients²⁰, and fetal cfDNA has been identified in urine during pregnancy²¹. The transrenal cfDNA in urine is much shorter than plasma cfDNA²¹, consistent with the expected size which can make it through the pores of the kidney. However, the transrenal cfDNA in urine is rapidly degraded by extracellular nucleases such as DNase I, which are 100-fold more active in urine than plasma. It was hypothesized that protecting the cfDNA in urine from nuclease attack could improve cancer detection and monitoring. Generating shorter cfDNA enabled channeling of more cfDNA for renal excretion, rather than elimination by Kupffer cells.

Studies have shown that patients with lupus produce autoantibodies against dsDNA, which are associated with shortening of cfDNA in plasma, due to the strong protective effective against extracellular nucleases²². Interestingly, the protective window against nuclease activity (30-50 bp) 23 coincides with the size distribution of cfDNA which can pass into urine²¹. Full length dsDNA antibodies are too large to pass through the pores of the kidneys and provoke autoimmune disorders such as lupus, when IgG-dsDNA complexes deposit in organs and activate the complement²⁴. However, antibody fragments are small enough to be rapidly filtered into the urine (as has been shown in drug delivery and medical imaging applications 2526), do not lead to complement activation²⁷, and can (in divalent form) confer protection against extracellular nucleases²³. Antibody fragments have also shown increased resistance to proteases and stability at extreme pH²⁸. Presented herein is the creation of a divalent nanobody which serves two purposes: (1) functions in concert with extracellular nucleases to produce more total cfDNA of small enough size to pass into urine, and (2) protects cfDNA in urine from further degradation by nucleases. Some fraction of the injected dose was rapidly excreted into the urine whereby it conferred the latter effect. This alone could improve cancer detection. However, it was reasoned that diverting more total cfDNA for renal excretion into urine would enable more complete sampling of cfDNA, from the entire blood volume, over longer durations of time. Additionally, a small antibody fragment, which can diffuse rapidly and deeply into organ tissue^{25,26}, would enable deeper sampling of cfDNA from regions that may ordinarily not spill cfDNA into the blood.

Experimental Approach. A llama was immunized with cfDNA to elicit a VHH response and candidate sequences directed to the cfDNA were recovered. The camelid antibodies were small, single-chain proteins of ˜15 kDa when produced as nanobodies. These elements are small by comparison to Fab or mAbs²⁷. The candidate nanobodies were expressed to confirm binding to dsDNA in yeast using existing methods for expression at high titers (>0.5 g/L). These nanobodies were expressed for their ability to confer nuclease resistance in a manner similar to that reported by Emlen et al²³. To evaluate the protective effect against nucleases, free and nucleosome-bound dsDNA were isolated from cell lines using micrococcal nuclease treatment to isolate mononucleosomes²⁹, and further proteinase-K treatment and DNA extraction was used to isolate free dsDNA¹. Then, a large volume of healthy donor plasma and urine was obtained, without DNase inhibitors such as EDTA, and was introduced to both free and nucleosome-bound dsDNA. The yield and size distribution of dsDNA resulting from exposure to plasma and urine was measured in a time course experiment in the presence of the candidate nanobodies, using methods previously described l. Additionally, previous work has identified several anti-dsDNA antibodies for detecting dsDNA. Lupus patient plasma that contains natural anti-dsDNA antibodies, and was tested for protection against nucleases. If a patient had protective antibodies, the heavy and light chain genes from collected B cells bound to labeled cfDNA were cloned from these patients. Mono- and divalent F(ab)s was generated from candidate antibodies. With appropriate candidate binders, divalent structures were created with linkers for the nanobodies or other constructs to test in vivo in healthy and tumor-bearing mice.

### References for Example 2

1. Adalsteins son, V. A. et al. Scalable whole-exome sequencing of cell-free DNA reveals high concordance with metastatic tumors. Nat. Commun. 8, 1324 (2017).
2. Bettegowda, C. et al. Detection of circulating tumor DNA in early- and late-stage human malignancies. Sci. Transl. Med. 6, 224ra24 (2014).
3. Kustanovich, A., Schwartz, R., Peretz, T. & Grinshpun, A. Life and death of circulating cell-free DNA. Cancer Biol. Ther. 20, 1057-1067 (2019).
4. Gauthier, V. J., Tyler, L. N. & Mannik, M. Blood clearance kinetics and liver uptake of mononucleosomes in mice. J. Immunol. 156, 1151-1156 (1996).
5. Cheng, T. H. T. et al. DNaseI Does Not Appear to Play a Major Role in the Fragmentation of Plasma DNA in a Knockout Mouse Model. Clinical Chemistry 64, 406-408 (2018).
6. Cutter, A. R. & Hayes, J. J. A brief review of nucleosome structure. FEBS Lett. 589, 2914-2922 (2015).
7. Nadano, D., Yasuda, T. & Kishi, K. Measurement of deoxyribonuclease I activity in human tissues and body fluids by a single radial enzyme-diffusion method. Clin. Chem. 39, 448-452 (1993).
8. Tavares, A. J. et al. Effect of removing Kupffer cells on nanoparticle tumor delivery. Proc. Natl. Acad. Sci. U.S.A 114, E10871-E10880 (2017).
9. Germain, M. et al. Priming the body to receive the therapeutic agent to redefine treatment benefit/risk profile. Sci. Rep. 8, 4797 (2018).
10. Qie, Y. et al. Surface modification of nanoparticles enables selective evasion of phagocytic clearance by distinct macrophage phenotypes. Sci. Rep. 6, 26269 (2016).
11. Kwon, E. J., Dudani, J. S. & Bhatia, S. N. Ultrasensitive tumour-penetrating nanosensors of protease activity. Nat Biomed Eng 1, (2017).
12. Chao, Y., Karmali, P. P. & Simberg, D. Role of carbohydrate receptors in the macrophage uptake of dextran-coated iron oxide nanoparticles. Adv. Exp. Med. Biol. 733, 115-123 (2012).
13. Du Clos, T. W. et al. Chromatin clearance in C57B1/10 mice: interaction with heparan sulphate proteoglycans and receptors on Kupffer cells. Clin. Exp. Immunol. 117, 403-411 (1999).
14. Chan, M. P. et al. DNase II-dependent DNA digestion is required for DNA sensing by TLR9. Nat. Commun. 6, 5853 (2015).
15. Lan, Y. Y., Londoilo, D., Bouley, R., Rooney, M. S. & Hacohen, N. Dnase2a deficiency uncovers lysosomal clearance of damaged nuclear DNA via autophagy. Cell Rep. 9, 180-192 (2014).
16. Kawane, K. et al. Chronic polyarthritis caused by mammalian DNA that escapes from degradation in macrophages. Nature 443, 998-1002 (2006).
17. Zhao, X., Li, L., Starr, T. K. & Subramanian, S. Tumor location impacts immune response in mouse models of colon cancer. Oncotarget 8, 54775-54787 (2017).
18. Castle, J. C. et al. Immunomic, genomic and transcriptomic characterization of CT26 colorectal carcinoma. BMC Genomics 15, 190 (2014).
19. Kaufman, D. P., Basit, H. & Knohl, S. J. Physiology, Glomerular Filtration Rate (GFR). in StatPearls (StatPearls Publishing, 2019).
20. Lu, T. & Li, J. Clinical applications of urinary cell-free DNA in cancer: current insights and promising future. Am. J. Cancer Res. 7, 2318-2332 (2017).
21. Tsui, N. B. Y. et al. High resolution size analysis of fetal DNA in the urine of pregnant women by paired-end massively parallel sequencing. PLoS One 7, e48319 (2012).
22. Chan, R. W. Y. et al. Plasma DNA aberrations in systemic lupus erythematosus revealed by genomic and methylomic sequencing. Proc. Natl. Acad. Sci. U.S.A 111, E5302-11 (2014).
23. Emlen, W., Ansari, R. & Burdick, G. DNA-anti-DNA immune complexes. Antibody protection of a discrete DNA fragment from DNase digestion in vitro. Journal of Clinical Investigation 74, 185-190 (1984).
24. Walport, M. J. Complement and systemic lupus erythematosus. Arthritis Res. 4 Suppl 3, S279-93 (2002).
25. Robinson, M. K. et al. Quantitative immuno-positron emission tomography imaging of HER2-positive tumor xenografts with an iodine-124 labeled anti-HER2 diabody. Cancer Res. 65, 1471-1478 (2005).
26. Cheng, Z. et al. 64Cu-labeled affibody molecules for imaging of HER2 expressing tumors. Mol. Imaging Biol. 12, 316-324 (2010).
27. Bannas, P., Hambach, J. & Koch-Nolte, F. Nanobodies and Nanobody-Based Human Heavy Chain Antibodies As Antitumor Therapeutics. Front. Immunol. 8, 1603 (2017).
28. Dekempeneer, Y. et al. Targeted alpha therapy using short-lived alpha-particles and the promise of nanobodies as targeting vehicle. Expert Opin. Biol. Ther. 16, 1035-1047 (2016).
29. Muthurajan, U. et al. In Vitro Chromatin Assembly. Methods Enzymol. 573, 3-41 (2016).

### Example 3—Use of Binders to Double-Stranded DNA (dsDNA) to Improve the Recovery of Cell-Free DNA (cfDNA)

Circulating tumor DNA (ctDNA) is a promising biomarker for precision oncology. It can be obtained noninvasively via a blood draw, provides information on clinically-actionable genetic variants, allows tumor DNA profiling from multiple metastatic sites to capture genetic heterogeneity, and can be performed serially to monitor response to treatment. It remains challenging to sample a sufficient quantity of ctDNA in a single blood draw, however, due to the often exceedingly low levels of tumor DNA in circulation, typically <100 pg/mL plasma.

The sensitivity of ctDNA testing depends on two factors: 1) the tumor fraction (TF), which is the fraction of ctDNA found in cell-free DNA (cfDNA), and 2) the cumulative mass of cfDNA sampled. The mass of cfDNA per 1 mL of plasma is around 10 ng, or the equivalent of 1,500 diploid genomes. In a typical 10 mL blood draw, 4 mL plasma or approximately 6,000 diploid genomes are recovered. When TF is below the reciprocal of this number (˜0.01-0.1%), the tumor genome may not be completely sampled in a blood draw. This sparsity makes it difficult to sensitively detect a given tumor mutation, and is especially relevant when testing in early stage disease or after definitive therapy, where initial TF is often well below 0.1%. At such low TF's, tumor mutation profiling becomes stochastic and unreliable, even when tracking hundreds of patient-specific tumor mutations genome-wide. It becomes challenging in such scenarios to distinguish whether absence of a mutation in a cfDNA sample indicates absence of cancer or simply lack of sampling of tumor DNA by chance.

Efforts to improve performance of ctDNA testing to date have focused on improving technical performance once a sample has been obtained, including integrating ctDNA features such as methylation patterns, fragment length and mutational signatures, and reducing sequencing errors. Such efforts have vastly improved the accuracy of ctDNA testing. At low TF's, however, tumor DNA may not even be sampled in a single blood draw (by partitioning) to be detected in current platforms. This fundamental limitation, due to the absence of tumor DNA molecules in the sample, cannot be overcome with deeper or more accurate sequencing.

A higher mass of cfDNA must be recovered to push beyond current ctDNA detection limits. More cfDNA enables detection at lower TF with the same sensitivity in sequencing. For example, recovery of a ten-fold higher mass of cfDNA (e.g., 200 ng vs 20 ng) would provide the same mass of ctDNA at 1% TF that 20 ng of cfDNA would provide at 10% TF (2 ng). A higher ctDNA mass would enable profiling of more of the tumor genome at higher depth. This would improve current ctDNA detection limits by making it possible to reliably profile more mutations, including those specific to individual patients, at lower TF.

This example addresses this fundamental limitation through use of a binder to double-stranded DNA (dsDNA), either nucleosome-bound or free dsDNA, and thereby improve the recovery of cfDNA.

The following mechanisms could be used in this Example: protection of cfDNA from digestion by nucleases in circulation and in tissue; shielding of cfDNA from uptake and clearance by macrophages; protecting a short length of dsDNA (a “footprint”) from nuclease digestion, and enabling these short fragments to be cleared into urine; improve the clearance of cfDNA into urine through the binding mechanism, for example by neutralizing the negative charge on dsDNA or displacing cfDNA from histones and condensing it.

By recovering a higher mass cfDNA, the performance of liquid biopsy tests for detection of ctDNA can be improved. These binders can be used in vivo to improve cfDNA recovery from biological fluids. They could also be used to manipulate the circulation or delivery of exogenous DNA in vivo (e.g., in gene therapy applications) or in vitro (e.g., in cell culture systems)

Several different compositions were considered for binders. These include engineered single-domain antibody dsDNA binders; anti-dsDNA antibodies; known dsDNA-binding proteins including members of the Sso7d family of proteins (a small, archaeal histone) and protamine sulfate.

Modifications of these binders were also considered, including divalent or multivalent constructs, fusion to Fc or other carrier proteins to improve circulation half-life, PEGylation and/or PASylation, and fusion to binders against cell-surface antigens on circulating cells (RBC's, WBC's) to concentrate cfDNA on cell surfaces as a compartment from which it can be recovered in a blood draw.

Protein binders can bind to dsDNA and protect it from digestion by nucleases. Such binders include antibodies, Sso7d, and protamine sulfate (FIGS. 8A-8B). Novel dsDNA-binding proteins were generated through directed evolution using a yeast display system. A single-domain antibody (“nanobody”) yeast display library was generated and a novel single-domain antibody binder with higher affinity to dsDNA was isolated that can be utilized for downstream applications (FIG. 9)

### Example 4—High-Sensitivity Liquid Biopsy Through Enhanced Cell-Free DNA Recovery

To enable precision oncology, prognostic and predictive biomarkers of disease recurrence and treatment response are needed. Circulating tumor DNA (ctDNA) is a promising biomarker for this purpose. 1-3 It can be obtained noninvasively via a blood draw, provides information on clinically-actionable genetic variants, allows tumor DNA profiling from multiple metastatic sites to capture genetic heterogeneity, and can be performed serially to monitor response to treatment. 2 It remains challenging to sample a sufficient quantity of ctDNA in a single blood draw, however, due to the often exceedingly low levels of tumor DNA in circulation, typically <100 pg/mL plasma.

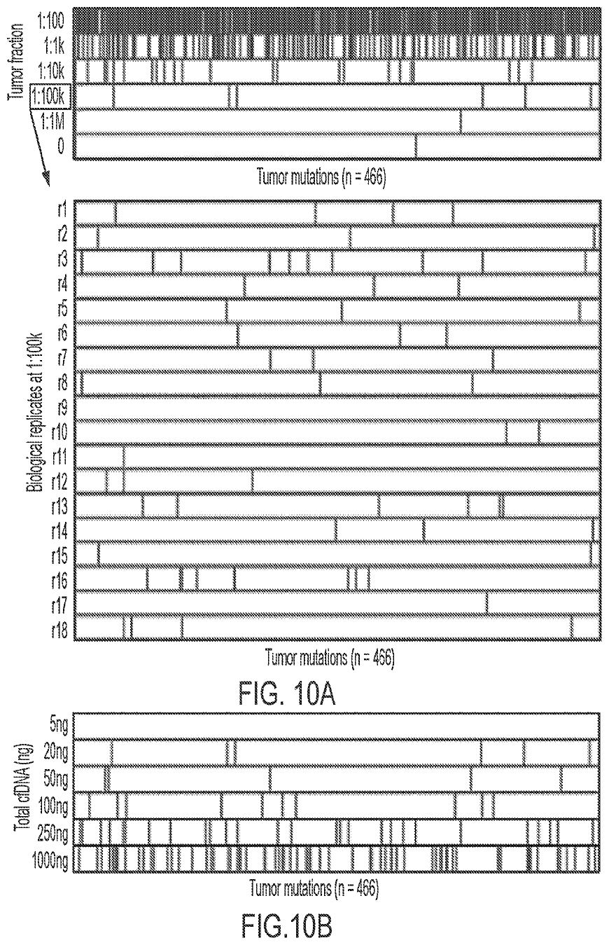

The sensitivity of ctDNA testing depends on two factors: 1) the tumor fraction (TF), which is the fraction of ctDNA found in cell-free DNA (cfDNA), and 2) the cumulative mass of cfDNA sampled. The mass of cfDNA per 1 mL of plasma is around 10 ng, or the equivalent of 1,500 diploid genomes. In a typical 10 mL blood draw, 4 mL plasma or approximately 6,000 diploid genomes are recovered.^{3,4} When TF is below the reciprocal of this number (˜0.01-0.1%), the tumor genome may not be completely sampled in a blood draw. This sparsity makes it difficult to sensitively detect a given tumor mutation, and is especially relevant when testing in early stage disease or after definitive therapy, where initial TF is often well below 0.1%.⁵ At such low TF's, tumor mutation profiling becomes stochastic and unreliable, even when tracking hundreds of patient-specific tumor mutations genome-wide (FIGS. 10A-10B). It becomes challenging in such scenarios to distinguish whether absence of a mutation in a cfDNA sample indicates absence of cancer or simply lack of sampling of tumor DNA by chance.

Efforts to improve performance of ctDNA testing to date have focused on improving technical performance once a sample has been obtained, including integrating ctDNA features such as methylation patterns, fragment length and mutational signatures, and reducing sequencing errors. 4-12 Such efforts have vastly improved the accuracy of ctDNA testing. At low TF's, however, tumor DNA may not even be sampled in a single blood draw (by partitioning) to be detected in current platforms. 3 This fundamental limitation, due to the absence of tumor DNA molecules in the sample, cannot be overcome with deeper or more accurate sequencing.

A higher mass of cfDNA must be recovered to push beyond current ctDNA detection limits.

More cfDNA enables detection at lower TF with the same sensitivity in sequencing. For example, recovery of a ten-fold higher mass of cfDNA (e.g., 200 ng vs 20 ng) would provide the same mass of ctDNA at 1% TF that 20 ng of cfDNA would provide at 10% TF (2 ng). A higher ctDNA mass would enable profiling of more of the tumor genome at higher depth. This would improve current ctDNA detection limits by making it possible to reliably profile more mutations, including those specific to individual patients, at lower TF (FIG. 10B).

This Example addresses this fundamental limitation through development of an intravenous molecular agent that can improve cfDNA recovery while preserving the convenience of a blood draw. Such an agent could vastly expand the use of ctDNA testing for cancer patients and help enable precision oncology.

cfDNA undergoes a complex life-cycle from release via apoptosis or necrosis, through circulation, cellular uptake and clearance. 13 In circulation, most cfDNA is nucleosome-bound, and is cleared by Kupffer cells^{14,15} or damaged and digested by circulating nucleases including DNase 1 and DNase 1L3.¹⁶ As a result of these processes, the half-life of cfDNA is only 1-2 hours in circulation. 13 It was hypothesized that inhibiting clearance mechanisms would extend the half-life of cfDNA and hence its cumulative levels in circulation. An increase in cfDNA levels would enable a higher mass to be recovered in a blood draw or urine sample, and in turn, mean higher absolute quantities of tumor DNA for the same TF, enabling more sensitive detection.

The performance of ctDNA assays was improved through development of methods to increase the amount of ctDNA recovered from biological fluids. Presented herein is the development of a molecular agent that can bind cfDNA and protect it from clearance. This agent increases the level of cfDNA in circulation. By increasing cfDNA levels while preserving the same TF, more ctDNA is recovered in a sample. This approach includes: 1) in vitro testing of dsDNA binders for their ability to protect DNA from nuclease digestion and cellular uptake, 2) development of novel dsDNA binders based on single-domain antibody scaffolds which have superior stability and manufacturability for in vivo use, and 3) in vivo testing of candidate binders to demonstrate increased cfDNA recovery and improved ctDNA detection. Through development of an agent that can improve cfDNA recovery, the performance of ctDNA tests is significantly improved.

Characterization of dsDNA binders for nuclease protection and inhibition of cellular uptake. dsDNA binders were characterized in vitro to establish whether they could protect dsDNA from nuclease digestion and from cellular uptake. By virtue of binding to dsDNA, some binders are able to block nucleases from digesting DNA and prevent cellular uptake through inhibition of association of free and mononucleosome-bound DNA with cell surface proteoglycans.

Engineering and production of novel dsDNA binding proteins using a single-domain antibody scaffold. Yeast surface display was used to engineer novel high-affinity binders to dsDNA. Multivalent and fusion constructs based on candidate binders were also generated. Binders for dsDNA were generated using scaffolds such as nanobodies which have desirable properties for in vivo use including temperature- and pH-stability.

Testing of candidate dsDNA binders in vivo in mouse xenograft models. The top candidates were tested in mouse xenograft models. The amount of cfDNA recovered, the TF, and the ability to extract genomic information from ctDNA with and without this agent were evaluated, while closely monitoring for any potential toxicity. The candidate binders led to an increase in cfDNA levels in circulation and improved the limit of detection for ctDNA.

Presented herein is a molecular agent that can be administered intravenously to bind and protect cfDNA, thereby increasing the total mass of cfDNA recovered in a sample. The agent described herein could be used in clinical settings where sensitive disease profiling is needed, such as in follow-up appointments after definitive cancer therapy or for molecular profiling at time of progression, in a similar way to the current use of intravenous iodine- and gadolinium-contrast agents for imaging studies in these scenarios.

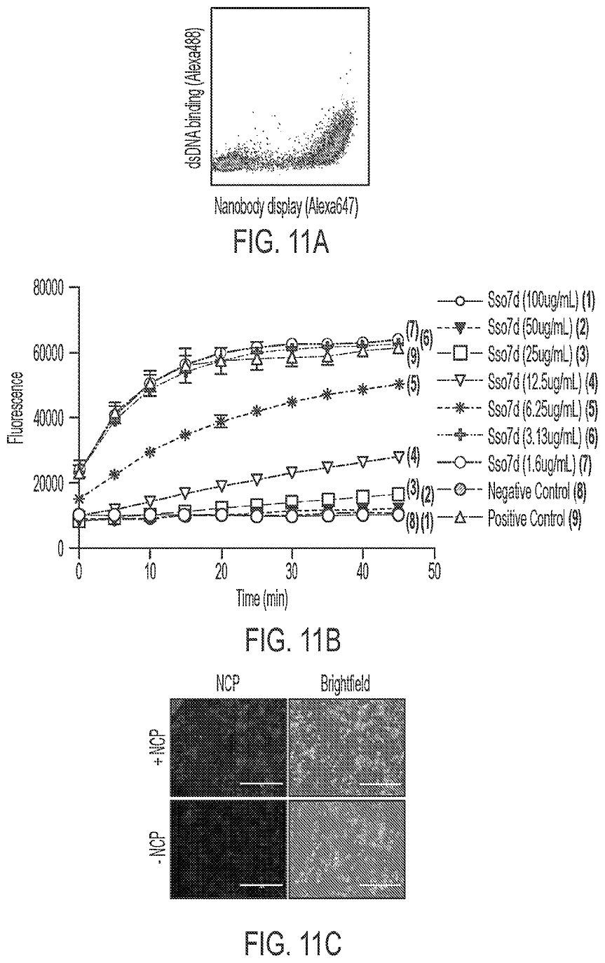

Establishment of a yeast surface display system for engineering binders to dsDNA. A yeast surface display system was established for engineering protein binders to dsDNA. 17-19 Yeast surface display offers several advantages, including a eukaryotic system for protein expression and the precision and sensitivity of fluorescence-activated cell sorting (FACS) for library screening. Display libraries were generated by introducing random mutations in known and novel DNA-binding proteins (DBPs), dsDNA binding was detected, and these libraries were screened for enhanced binding to dsDNA (FIG. 11A).

An in vitro system for nuclease protection testing. An in vitro system was established using a fluorescently tagged dsDNA probe with a quencher to detect the ability of candidate DBPs to protect dsDNA from nuclease digestion in real-time (FIG. 11B). This assay was benchmarked against a digestion time series of dsDNA oligonucleotides with recombinant DNase I and demonstrated close agreement. Using this system, numerous candidates were screened in a parallel fashion for their ability to protect dsDNA.

An in vitro system for dsDNA and nucleosome uptake. To test the ability of a candidate binder to inhibit cellular uptake of free and nucleosome-bound dsDNA in vitro, a cell culture system was established using the RAW264.7 macrophage cell line (FIG. 11C). Cellular uptake of biotin-tagged mononucleosomes and dsDNA was detected using epifluorescence microscopy and was also quantified on a fluorescent plate reader, enabling screening for reagents that inhibit uptake.

Mouse xenograft models with ctDNA fingerprints. A tumor fingerprint assay was developed for the CT26 colorectal carcinoma cell line, which was used to test for ctDNA in plasma and urine from CT26 mouse xenograft models.

Characterization of dsDNA binders for nuclease protection and inhibition of cellular uptake.

DBPs that can protect cfDNA in vivo protect cfDNA from nuclease digestion and from cellular uptake. Screening assays in vitro were used to test candidates for these properties prior to in vivo testing.

The ability of DBP's—both known proteins and novel ones generated above—to protect dsDNA from nuclease digestion using a previously-established plate-based fluorescence assay was evaluated Known DBP's that can be tested included anti-dsDNA antibodies (some commercially available, e.g. Abcam ab27156),^{20,21} members of the protamine family of DBP's, 22 and a family of small DNA-binding archaeal proteins including Sso7d.²³⁻²⁵ A commercially available dsDNA probe conjugated to a HEX fluorophore and a quencher was incubated with recombinant DNase 1 and DNase 1L3 (the predominant nucleases in circulation). 16 Successful digestion of the linking dsDNA separated fluorophore and quencher, leading to a fluorescent signal that was measured on a plate reader. This assay can screen numerous candidate binders over a range of concentrations in a high-throughput format on 96-well plates, allowing for rapid characterization of nuclease protection ability. The results were verified with a nuclease digestion time-series of oligonucleotides. The size profile of protected DNA fragments was also characterized. DNA fragments up to 50-bp are small enough to be filtered into and concentrated in urine, which is another source of cfDNA.²⁶

A RAW264.7 macrophage cell culture system was used to test the ability of binders to inhibit cellular uptake of dsDNA and mononucleosomes. Cells were activated with lipopolysaccharide (LPS) at 100 ng/mL for 2 hours and then incubated with candidate binders and biotin-tagged free- or nucleosome bound-dsDNA (EpiCypher) for 2 hours. Cells were washed, fixed, and labelled with streptavidin-fluorophore conjugates. Fluorescence microscopy was used to quantify signal within cells. This Example was benchmarked in 96-well plate format with signal measurement using a plate reader to enable high-throughput evaluation of binders.

These assays were used to prioritize binders for advancement to in vivo testing. The criteria for advancement were: 1) binders that protect dsDNA from nuclease digestion without promoting cellular uptake, 2) binders that inhibit cellular uptake with or without a protective effect on nuclease digestion, and 3) binders that protect DNA in a ˜50 bp window, enabling filtration into urine.

Engineering and production of novel dsDNA binding proteins using a single-domain antibody framework.

The ideal DBP is small (to penetrate deeply within tissue), stable, with a high affinity to dsDNA but with little sequence specificity. Multiple DBP's have already been identified without all of the properties desired for a cfDNA protector. In addition to testing known DBP's, novel DBP's were engineered with the desired properties.

Yeast surface display was used to generate novel DBP's. A single domain nanobody (VHH) framework was used, which has several desirable properties including small size of 12-15 kDa, stability in a range of pH and temperatures, low immunogenicity, and ease of manufacturing in prokaryotic and yeast systems. 27 A VHH display library of putative dsDNA binders was constructed. A display library was also developed based on the framework of the highly thermostable archaeal DBP Sso7d 25 that was screened in parallel. Multiple rounds of FACS sorting against biotinylated dsDNA oligonucleotides of varying sequence and GC content were used to isolate the highest-affinity binders that are relatively sequence agnostic. High affinity (K_{d}<50 nM) binders were isolated via affinity maturation through mutagenesis and library screening. High affinity binders were expressed in Pichia pastoris, a host organism used for rapid production and purification of heterologous proteins at high titer.²⁸⁻³⁰ Expressed product was purified from culture supernatant using fast-protein liquid chromatography (FPLC) and characterized with mass spectrometry (MS). A c-myc tag was incorporated on all products to enable detection in in vivo samples below. DNA-binding activity of products will be determined qualitatively with electrophoretic mobility shift assays (EMSA) and measured quantitatively with biolayer interferometry (BLI). Bivalent constructs and Fc-fusion products were also generated from binders identified in this screen to improve half-life and affinity for in vivo testing. Products were screened for desired properties using in vitro assays above.

Testing of candidate dsDNA binders in vivo in mouse xenograft models.

DBPs were tested in mouse xenograft models to determine whether if they can boost cfDNA recovery in vivo, and whether that boost translates to higher ctDNA detection sensitivity.

Flank xenograft models were used to test candidate binders. The top candidate constructs from above were expressed at high titer and purified and characterized to ensure sufficient quality for in vivo testing. Bi-flank models were established by injecting 5×10⁶-1×10⁷ tumor cells subcutaneously and allowing tumors to grow until approximately 1500-2000 mm³ in size. Blood was drawn in 70 μL aliquots at baseline and in a time series after injection of DBP or PBS control (i.e., at 10 min., 30 min., 1 hr., and 2 hr. post-injection). Urine was collected over 1 hr. to assess for any differences in urinary cfDNA. For a subset of mice, a terminal bleed sample was collected via cardiac puncture, and liver, kidneys and spleen were harvested for fixation and histology to detect any immediate toxic effects and assess for organ-specific uptake of DBP. Mice that were not sacrificed were monitored for development of toxicities, primarily renal and hepatic.

cfDNA was extracted from blood and urine samples and quantified. For those samples with sufficient cfDNA, a library was constructed, hybridization captured using CT26 probe panels, and next generation sequencing was performed to quantify ctDNA yield and TF using established protocols. 5 A small aliquot of plasma and urine was set aside for ELISA assays to quantify amount of binder and confirm binding to dsDNA. Development of an immune reaction to the candidate agent was assessed through ELISA assays of mouse plasma following exposure.

### References for Example 4

1. Wan, J. C. M. et al. Liquid biopsies come of age: Towards implementation of circulating tumour DNA. Nature Reviews Cancer 17, 223-238 (2017).
2. Cescon, D. W., Bratman, S. V., Chan, S. M. & Siu, L. L. Circulating tumor DNA and liquid biopsy in oncology. Nat Cancer 1, 276-290 (2020).
3. Heitzer, E., Hague, I. S., S Roberts, C. E. & Speicher, M. R. Current and future perspectives of liquid biopsies in genomics-driven oncology. Nature Reviews Genetics (2019) doi:10.1038/s41576-018-0071-5.
4. Newman, A. M. et al. Integrated digital error suppression for improved detection of circulating tumor DNA. Nat Biotechnol 34, 547-555 (2016).
5. Parsons, H. A. et al. Sensitive Detection of Minimal Residual Disease in Patients Treated for Early-Stage Breast Cancer. Clin Cancer Res (2020) doi:10.1158/1078-0432.CCR-19-3005.
6. Bettegowda, C. et al. Detection of Circulating Tumor DNA in Early- and Late-Stage Human Malignancies. Sci Transl Med 6, 224ra24 (2014).
7. Chabon, J. J. et al. Integrating genomic features for non-invasive early lung cancer detection. Nature 1-7 (2020) doi:10.1038/s41586-020-2140-0.
8. Liu, M. C. et al. Sensitive and specific multi-cancer detection and localization using methylation signatures in cell-free DNA. Annals of Oncology 0, (2020).
9. Wan, J. C. M. et al. ctDNA monitoring using patient-specific sequencing and integration of variant reads. Science Translational Medicine 12, (2020).
10. Cristiano, S. et al. Genome-wide cell-free DNA fragmentation in patients with cancer. Nature 570, 385-389 (2019).
11. Newman, A. M. et al. An ultrasensitive method for quantitating circulating tumor DNA with broad patient coverage. Nature Medicine 20, 548-554 (2014).
12. Zviran, A. et al. Genome-wide cell-free DNA mutational integration enables ultrasensitive cancer monitoring. Nat Med (2020) doi:10.1038/s41591-020-0915-3.
13. Kustanovich, A., Schwartz, R., Peretz, T. & Grinshpun, A. Life and death of circulating cell-free DNA. Cancer Biol Ther 20, 1057-1067 (2019).
14. Du Clos, T. W. et al. Chromatin clearance in C57B1/10 mice: interaction with heparan sulphate proteoglycans and receptors on Kupffer cells. Clin Exp Immunol 117, 403-411 (1999).
15. Gauthier, V. J., Tyler, L. N. & Mannik, M. Blood clearance kinetics and liver uptake of mononucleosomes in mice. The Journal of Immunology 156, 1151-1156 (1996).
16. Han, D. S. C. et al. The Biology of Cell-free DNA Fragmentation and the Roles of DNASE1, DNASE1L3, and DFFB. The American Journal of Human Genetics 106, 202-214 (2020).
17. Boder, E. T. & Wittrup, K. D. Yeast surface display for screening conlbinatorial polypeptide libraries. 15, 5 (1997).
18. Chao, G. et al. Isolating and engineering human antibodies using yeast surface display. Nat Protoc 1, 755-768 (2006).
19. Van Deventer, J. A. & Wittrup, K. D. Yeast Surface Display for Antibody Isolation: Library Construction, Library Screening, and Affinity Maturation. in Monoclonal Antibodies: Methods and Protocols (eds. Ossipow, V. & Fischer, N.) 151-181 (Humana Press, 2014). doi:10.1007/978-1-62703-992-5_10.
20. Barbas, S. M. et al. Human autoantibody recognition of DNA. Proc Natl Acad Sci USA 92, 2529-2533 (1995).
21. Wellmann, U. et al. The evolution of human anti-double-stranded DNA autoantibodies. PNAS 102, 9258-9263 (2005).
22. Oliva, R. & Mezquita, C. Marked differences in the ability of distinct protamines to disassemble nucleosomal core particles in vitro. Biochemistry 25, 6508-6511 (1986).
23. Agback, P., Baumann, H., Knapp, S., Ladenstein, R. & Hard, T. Architecture of non specific protein-DANN interactions in the Sso7d-DANN complex. Nature structural biology 5, 579-84 (1998).
24. Shehi, E. et al. Thermal Stability and DNA Binding Activity of a Variant Form of the Sso7d Protein from the Archeon Sulfolobus solfataricus Truncated at Leucine 54^{†}. Biochemistry 42, 8362-8368 (2003).
25. Kalichuk, V. et al. The archaeal “7 kDa DNA-binding” proteins: extended characterization of an old gifted family. Scientific Reports 6, 37274 (2016).
26. Markus, H. et al. Analysis of recurrently protected genomic regions in cell-free DNA found in urine. Science Translational Medicine 13, (2021).
27. Muyldermans, S. Nanobodies: Natural Single-Domain Antibodies. Annu. Rev. Biochem. 82, 775-797 (2013).
28. Crowell, L. E. et al. On-demand manufacturing of clinical-quality biopharmaceuticals. Nat Biotechnol 36, 988— 995 (2018).

### Example 5—Liposomal Contrast Agents Increase Circulating Tumor DNA Recovery and Enhance Sensitivity of Liquid Biopsies

Circulating tumor DNA (ctDNA) shed by cancer cells is a promising non-invasive biomarker for cancer diagnostics as it is easily accessible via a blood draw and carries valuable signatures of disease^{4,5}. As such, ctDNA could have broad applications across all stages of cancer management, from cancer screening to monitoring to detection of recurrence after curative-intent therapy. However, current limits of detection (LOD) often preclude detection of ultra-low levels of ctDNA, such as in early stage disease or after definitive treatment. As a result, sensitivity of screening tests remains low for early-stage disease and a significant fraction of patients who test negative for minimal residual disease (MRD) still experience recurrence. For example, tumor-informed tests using ctDNA mutations can only detect 47% of stage I tumors and tumor-agnostic methylation tests only have 18% sensitivity for stage I tumors^{18,19}, while 25% of MRD-negative patients still experience recurrence^{12,20}. To enable application of ctDNA assays in these clinical scenarios, improvements in sensitivity are needed.

Current limitations in sensitivity are driven by the error rate of next generation sequencing and the low quantity of tumor DNA in a typical blood sample. On one hand, sequencing errors make it challenging to distinguish true mutations from sequencing noise as ctDNA levels decrease. On the other hand, low tumor DNA quantity introduces significant stochasticity which compromises sensitivity. As the tumor fraction (TF), which is the fraction of tumor-derived DNA in a cell-free DNA (cfDNA) sample, decreases, a given tracked mutation is less likely to be represented in the cfDNA fragments sampled in a blood draw, and is therefore less likely to be detected, and no degree of sequencing accuracy can overcome this fundamental limitation.

Methods to improve the sensitivity of ctDNA detection to date have been focused downstream of blood sampling and include suppressing error rates, tracking multiple somatic variants, or tracking other features such as methylation and fragmentation patterns. Although these methods have led to improvements in LOD, they remain hampered by the limitation of low input ctDNA. As such, they rely on tumors having sufficiently high mutation rates or a specific distribution of mutations to enable high detection accuracy, and are difficult to generalize to all patients. Furthermore, the potential LOD improvement through current methods has a ceiling set by the finite number of trackable variants in the genome¹². To push beyond current detection limits, other approaches to sample more ctDNA safely are needed.

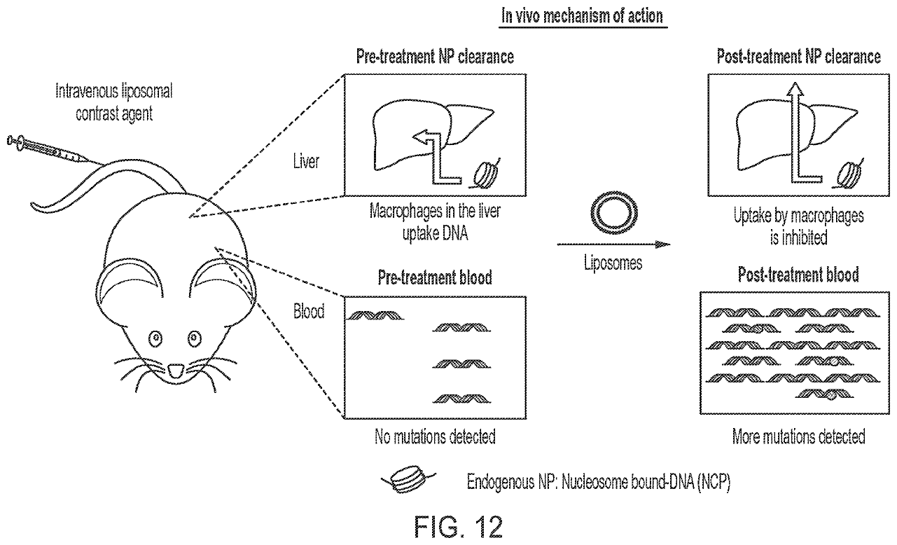

Therefore, a means to address the fundamental limitation of low input ctDNA was addressed by intervening directly on the clearance of cfDNA, upstream of blood sampling, in order to transiently increase cfDNA levels. Although plasma cfDNA consists of a mixture of DNA fragments, a large proportion of cfDNA molecules circulate wrapped around histone proteins and form so-called nucleosome core particles (NCPs). NCPs are approximately 11 nm in diameter²³ and as such are subject to similar clearance routes to other exogenously administered nanoparticles and are primarily cleared by liver-resident macrophages called Kupffer cells^{10,21}. Conceptualizing mononucleosomes as endogenous, circulating nanoparticles, a method has been developed to temporarily increase the amount of cfDNA that can be recovered from a blood draw by using a decoy liposomal nanoparticle to inhibit the clearance of cfDNA from the circulation (FIG. 12). This agent is first demonstrated to saturate the uptake pathways of macrophages and decreases the uptake of mononucleosomes in vitro. Treatment with this agent is then shown to increase the half-life of exogenous mononucleosomes in vivo. Finally, this approach is tested in a mouse tumor model to demonstrate that it enables significantly higher recovery of ctDNA from a blood draw.

In Vitro NCP Uptake Inhibition with Liposomes

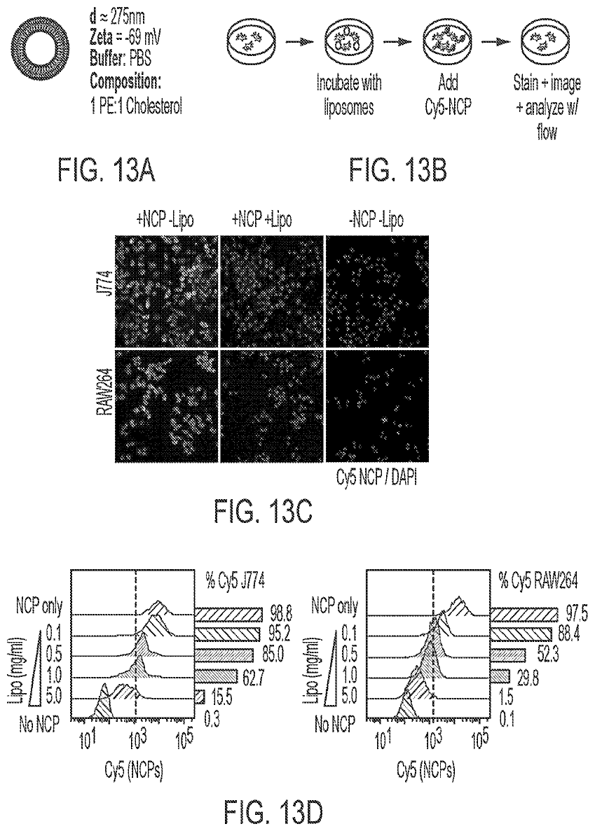

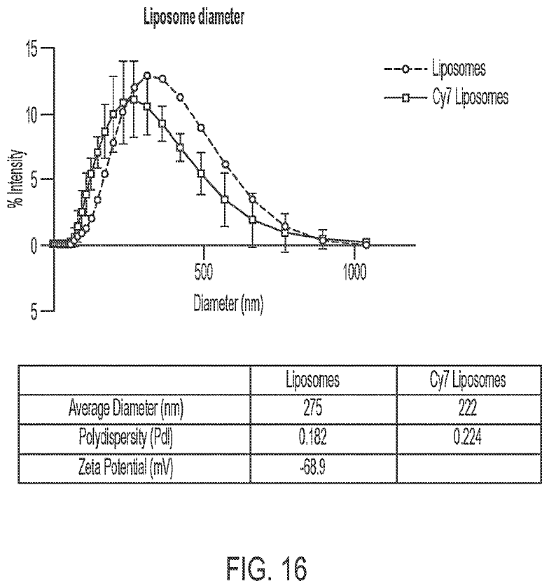

Liposomes comprising phosphoethanolaminein (PE) and cholesterol were first synthesized in PBS buffer (FIG. 13A, FIG. 16) by modifying previously reported methods¹³. This formulation was selected as its physico-chemical attributes had been optimized to preferentially occupy Kupffer cells (KCs), responsible for the majority of NCP clearance, without signs of acute toxicity¹⁴. By administering liposomes prior to a range of nanomedicines, others have previously shown that liposomes transiently occupy KCs and limit hepatic accumulation of administered nanomedicines, increasing their blood bioavailability^{13,14}.

It was hypothesized that the same decoy liposome formulation might also prevent KC uptake of NCPs and thus increase their blood bioavailability. To test this, an in vitro assay was designed to assess if pre-treatment of two different macrophage cell lines with the decoy liposome particle would affect NCP uptake. Both RAW264 and J774 cells were tested, as these are two of the most extensively used macrophage cell lines for investigating nanoparticle uptake by the reticuloendothelial system.

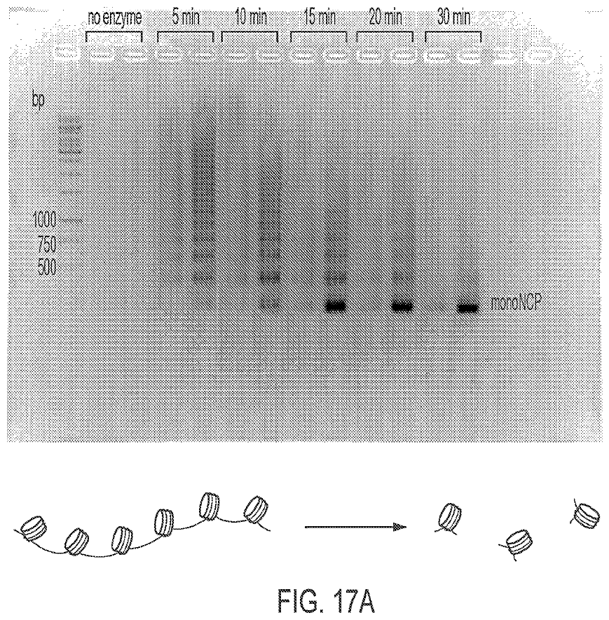

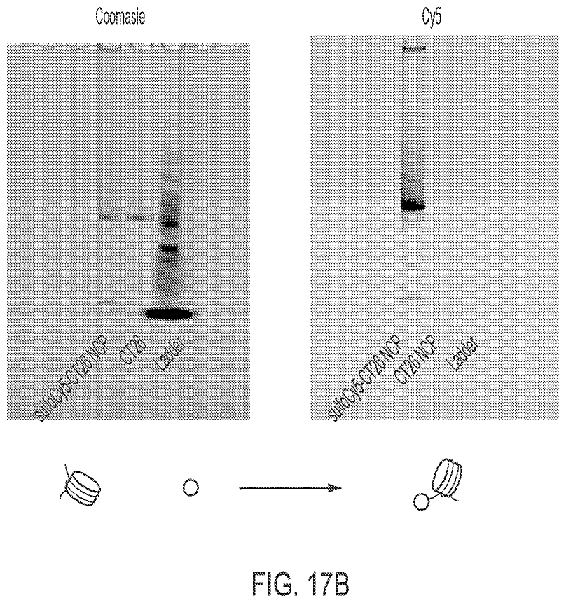

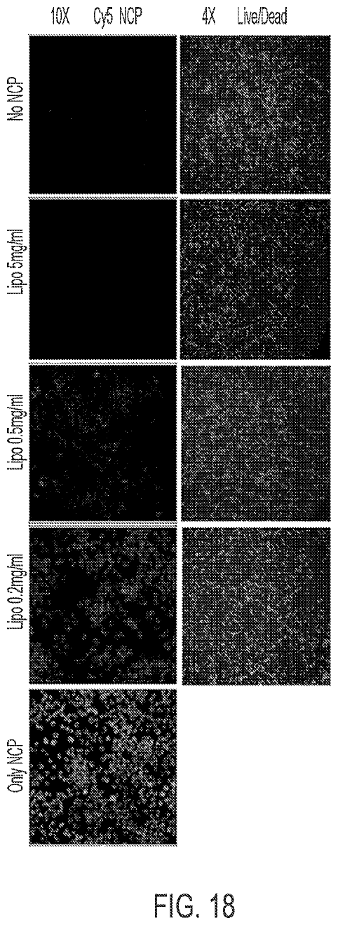

Following pre-treatment of cells with liposomes at a range of concentrations, Cy5-labelled NCPs were added (FIG. 17) and their uptake by macrophages was quantified (FIG. 13B). Epifluorescence imaging was performed to visualize the localization of Cy5-positive NCPs as well as flow cytometry analysis to quantify the distribution of Cy5 positivity across the cell population. It was observed that both macrophage cell lines efficiently took up Cy5-labelled NCPs (FIG. 13C). However, upon pre-incubation of cells with liposomes, NCP uptake as quantified by Cy5-positivity decreased in a dose-dependent manner for both cell lines (FIG. 13D). The percent of Cy5-positive cells recorded decreased from 95.2% to 85% to 62.7% to 15.5% for J774 cells and from 88.4% to 52.3% to 29.8% to 1.5% for RAW264 cells as liposome concentrations increased from 0.1 to 0.5 to 1 to 5 mg/ml. Notably, the condition with the highest liposome concentration achieved an 84.5% and a 98.5% reduction in NCP uptake by J774 and RAW264 cells, respectively, compared to untreated cells. Importantly, cell viability was not compromised by liposome treatment as demonstrated using a live-dead dye (FIG. 18).

Plasma NCP Half-Life Extension Investigation with Liposomes

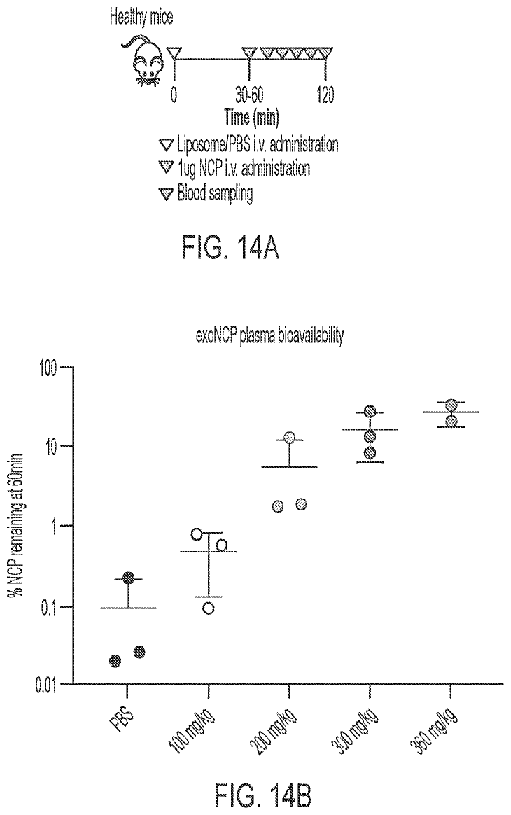

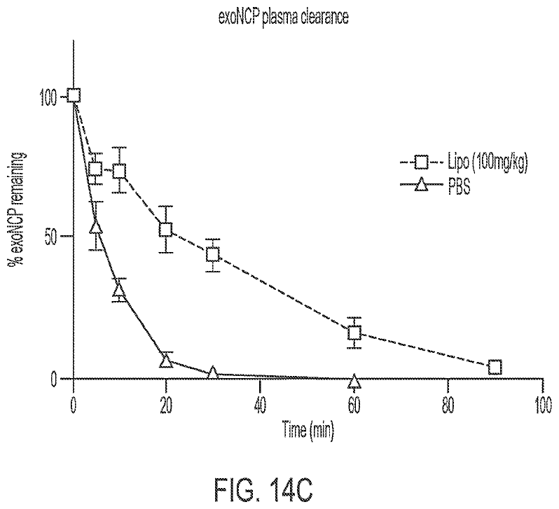

Having established that decoy liposomes could inhibit NCP uptake by macrophages in vitro, a healthy mouse model was then tested to determine whether liposome pre-treatment in mice could inhibit the clearance and thus extend the half-life of NCPs in blood. To this end, mice were pre-treated with a range of liposome doses and subsequently administered exogenous commercial NCPs (exoNCPs) carrying a Widom601 DNA sequence¹⁷ that does not align to the mouse genome and can be quantified via qPCR (FIG. 14A). Importantly, these exogenously dosed exoNCPs are only subject to clearance in vivo and thus serve as a useful model to decouple the effects of cfDNA shedding and clearance on plasma cfDNA yields.

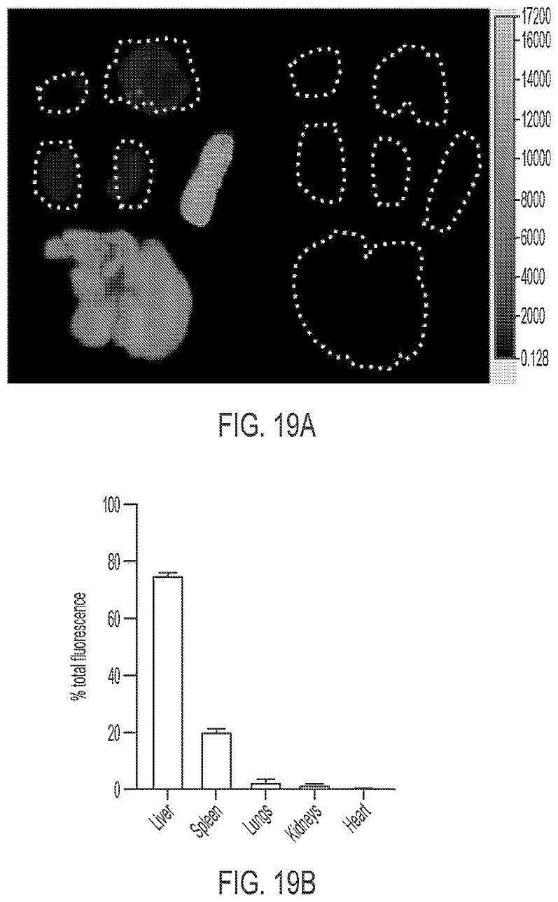

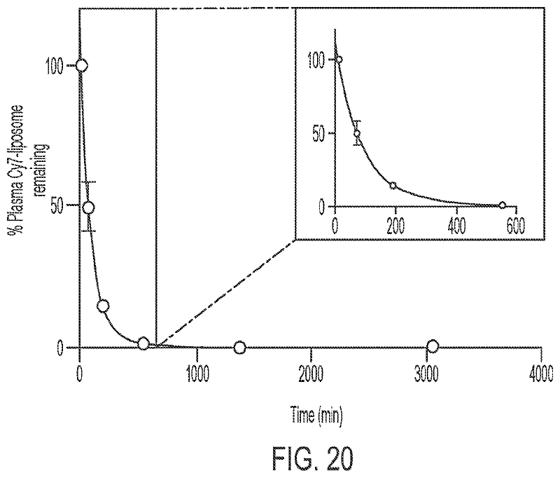

Consistent with previous studies, in vivo liposomes mostly accumulated in the liver and spleen 1 hour after administration (FIG. 19). Quantified Widom601 levels in plasma were consistent with decreased exoNCP clearance upon liposome treatment, as the percent of Widom601 remaining in plasma 60 min. after NCP administration increased in a dose-dependent manner, from on average 0.1% to 0.5% to 5.7% to 16.5% to 26.8% at 0, 100, 200 and 360 mg/kg, respectively (FIG. 14B). For mice treated with the highest liposome dose, the percent of Widom601 remaining was more than 100-fold higher than levels detected for PBS-treated (negative control) mice. The half time of NCPs upon treatment with the lowest dose, 100 mg/kg of liposomes, was quantified. When liposomes were administered 30 min. prior to NCP administration, half-life extension of exoNCPs was observed from 5.9 min. to 25.3 min. in liposome vs. PBS treated mice (one-phase decay model, R squared=0.99 and 0.96, respectively) (FIG. 14C).

Plasma Cell-Free DNA Profiling in Tumor-Bearing Mice with Liposomal Contrast Agent

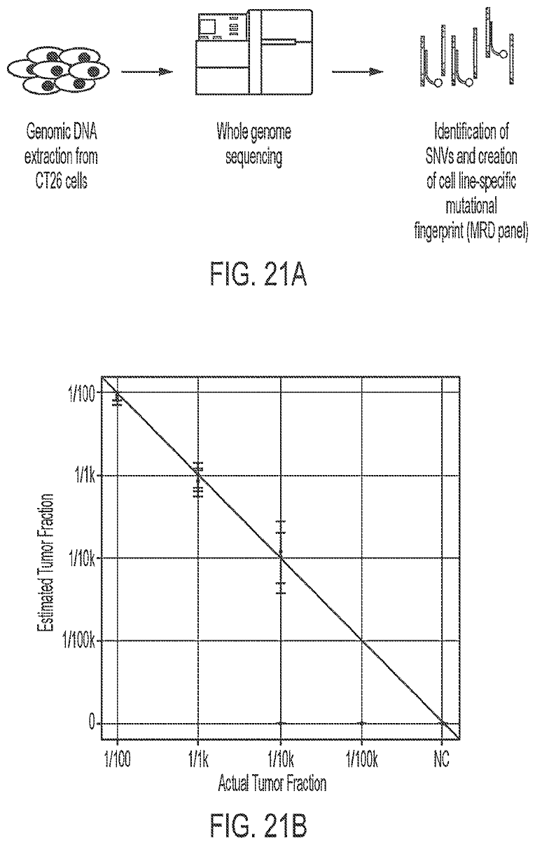

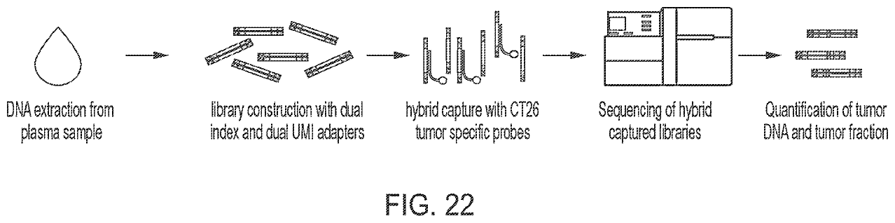

Having demonstrated that in vivo liposome treatment increases the bioavailability of exoNCPs in plasma, it was hypothesized that administering liposomes prior to blood sampling would increase the yield of ctDNA in a blood draw and thus improve the sensitivity of ctDNA testing. To test this, a flank tumor model of the colorectal cell line CT26 was tested. A ctDNA assay was designed that tracked 98 mutations (see Methods) and utilized duplex-sequencing for error suppression^{12,22}. This assay could reliably detect single mutant duplexes and quantify tumor fractions down to 1e-4 (FIG. 21, FIG. 22). A list of mutations for the CT26 cell line has been previously published, upon which readily a mutational probe panel could be designed to assess ctDNA levels in this model. In addition, it had also been shown that this model was capable of shedding DNA into circulation and was thus suitable for ctDNA testing.

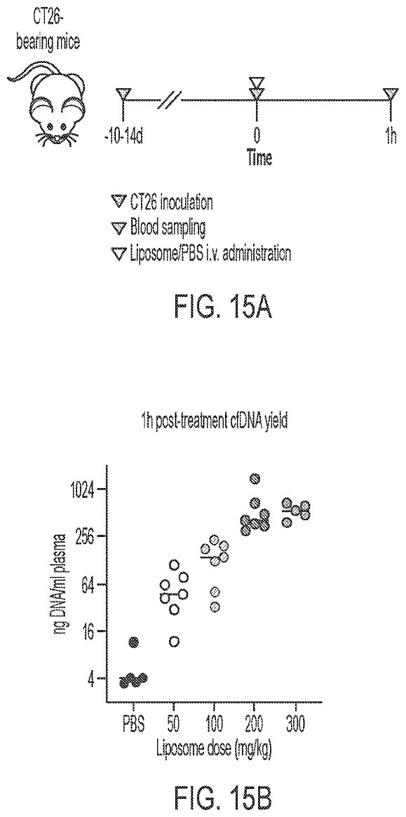

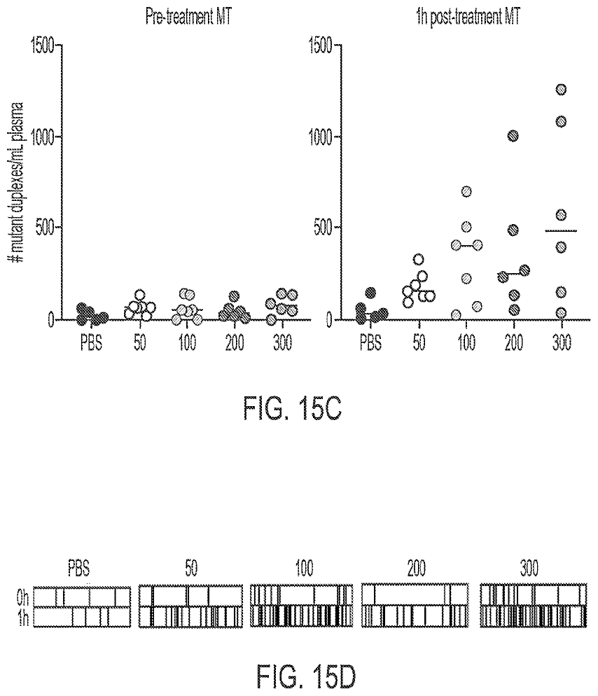

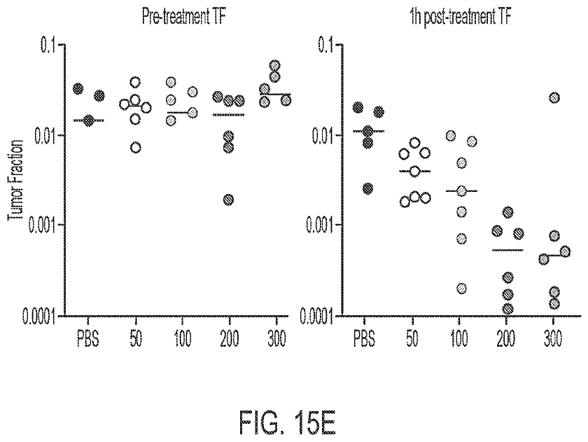

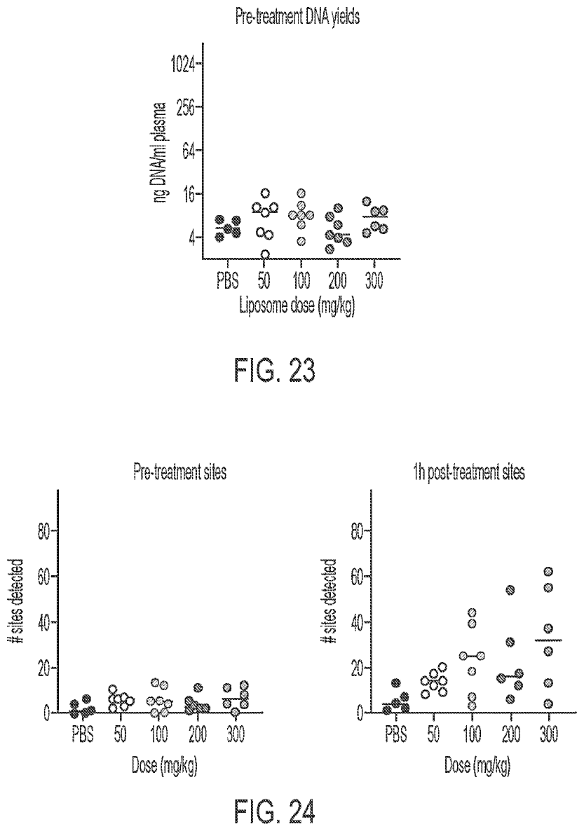

Mice were inoculated with CT26 tumors and treated with liposomes at different doses or PBS (negative control), and blood was collected prior to and 1 hour after treatment (FIG. 15A). Despite having observed the greatest accumulation of exoNCPs at the highest liposome dose (360 mg/kg), the minimum effective dose of liposomes that would improve ctDNA testing while minimizing potential side effects was determined. Thus, mice were treated with four different liposome doses (50, 100, 200 and 300 mg/kg) or PBS. Importantly no statistical differences were observed for the pre-treatment total cfDNA yields between different mouse groups (FIG. 23). By contrast, 1 hour after liposome administration, plasma cfDNA yields increased significantly in a dose-dependent manner (FIG. 15B). Notably, while pre-treatment cfDNA yields across all groups were comparable to yields typically observed in humans (average of 7.6+/−2.2 ng/ml plasma in this cohort vs. 5-10 ng/ml plasma typically in humans), cfDNA yields 1 hour after liposome administration increased up to 140-fold relative to pre-treatment yields, reaching cfDNA concentrations above 600 ng/ml. More specifically, the average cfDNA concentrations in plasma 1 hour after treatment administrations were 5+/−3, 54+/−33, 136+/−74, 437+/−135 and 549+/−116 ng/ml for PBS, 50 mg/kg, 100 mg/kg, 200 mg/kg and 300 mg/kg treated mice, respectively, and follow the same trend as the exoNCP bioavailability results in healthy mice (FIG. 14B).

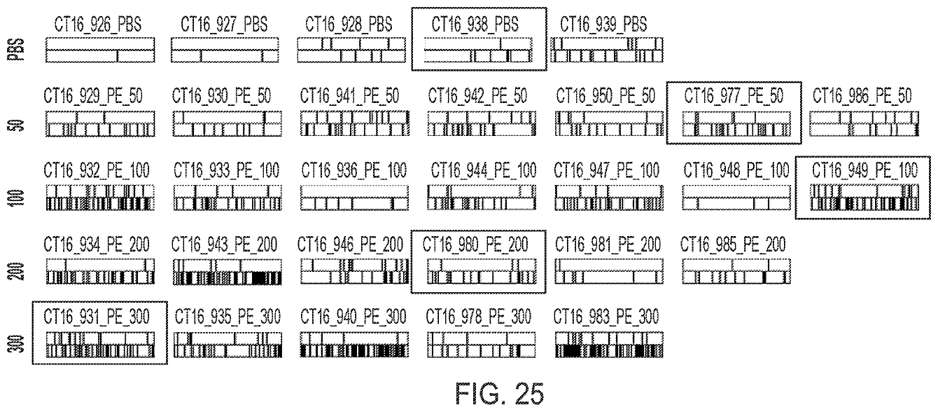

Next the custom CT26 mutational profiling assay was employed to quantify tumor-derived cfDNA (ctDNA) in plasma. It was observed that the results followed a similar trend as those observed for total cfDNA yield. While the number of mutant DNA duplexes (MT) detected pre-treatment remained low (54+/−22 MT/ml plasma) across all groups, the number of mutant duplexes detected in blood sampled 1 hour after liposome administration drastically increased in comparison with that of the PBS treated mice (FIG. 15C). When compared to pre-treatment samples, the number of MT detected increased up to 9.3-fold (mean 6.0), 23.7 (mean 10.1) and 23.1 (mean 10.3) in mice pre-treated with 100 mg/kg, 200 mg/kg and 300 mg/kg respectively. The recovery of more MT 1 hour after liposome treatment enabled the detection of additional mutational sites in the 98-probe panel (FIG. 24, FIG. 25) from on average 5+/−5 to 13+/−3 to 23+/−13 to 23+/−19 to 33+/−18 sites in PBS, 50 mg/kg, 100 mg/kg, 200 mg/kg and 300 mg/kg, respectively. FIG. 15D shows the mutational sites detected for one representative mouse per group. Finally, when examining tumor fractions, pre-treatment tumor fractions were observed to be comparable between all groups. While PBS treatment had a minor impact on tumor fractions, liposome treatment significantly decreased tumor fractions in a dose dependent manner, with lower tumor fractions at higher doses. (FIG. 15E). However, despite the decrease in tumor fraction, the increase in total amount of cfDNA upon liposome treatment enabled recovery of up to 10-fold more ctDNA per blood draw. Taken together, these results illustrate the utility of decoy liposomes to increase cfDNA levels in tumor-bearing mice, improving both the likelihood to detect tumor-derived DNA molecules as well as the molecular profiling of tumors.

The Effect of Liposome Contrast Agent is Transitory with Little or No Toxicity

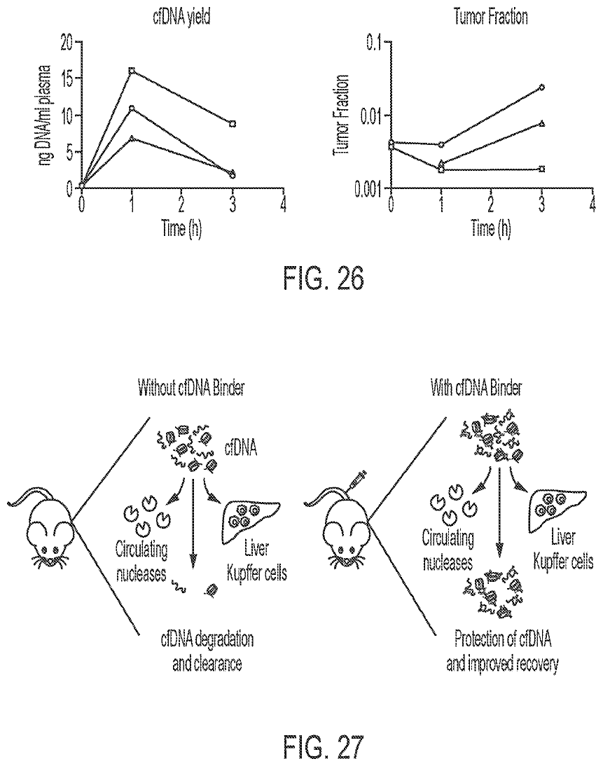

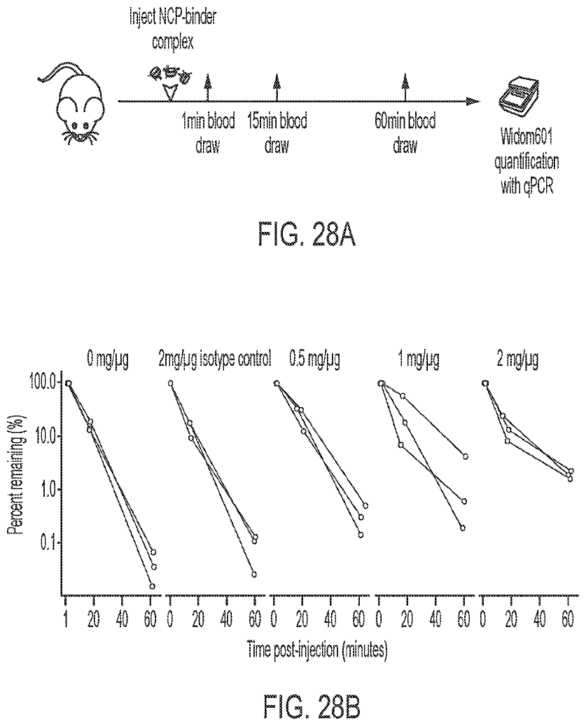

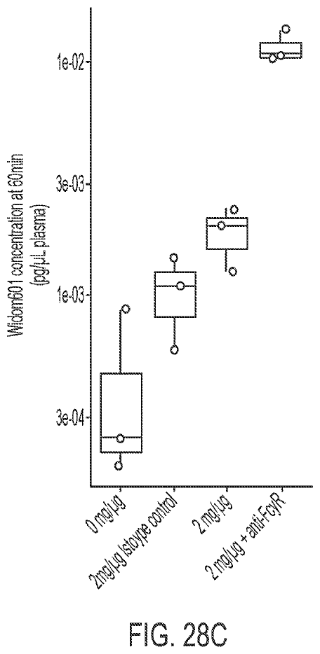

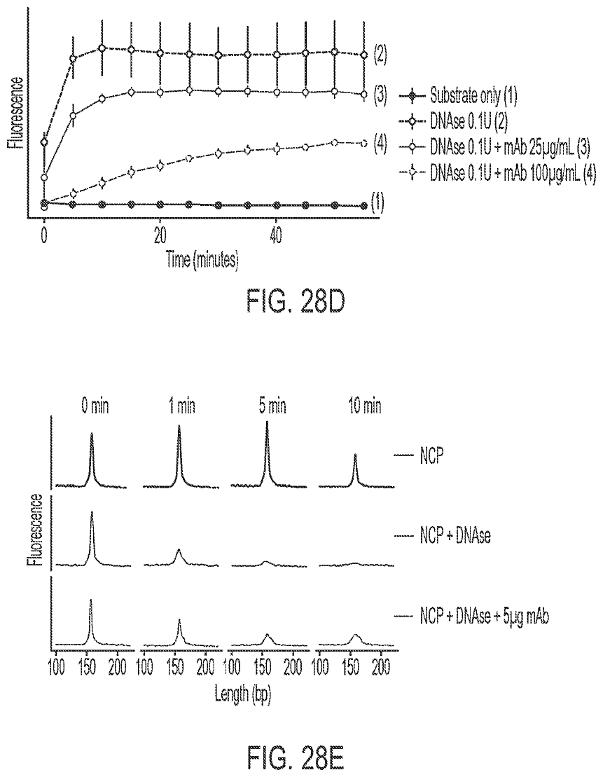

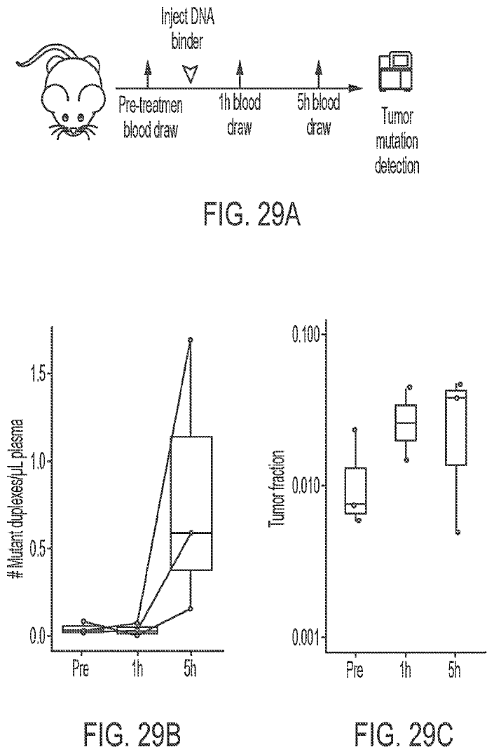

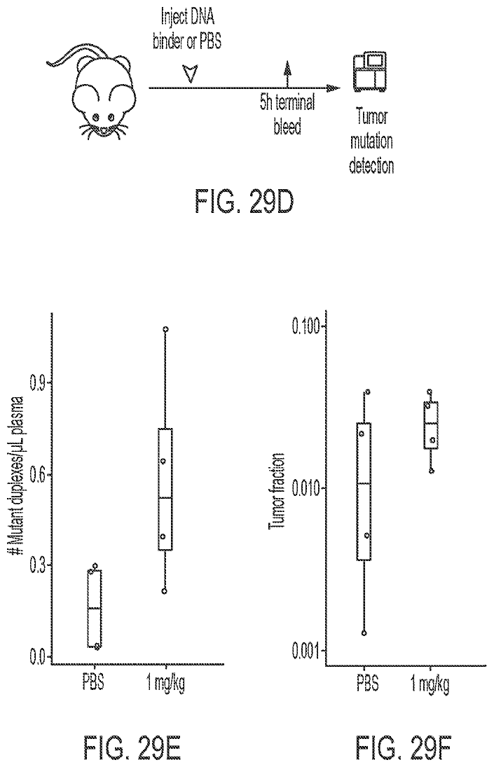

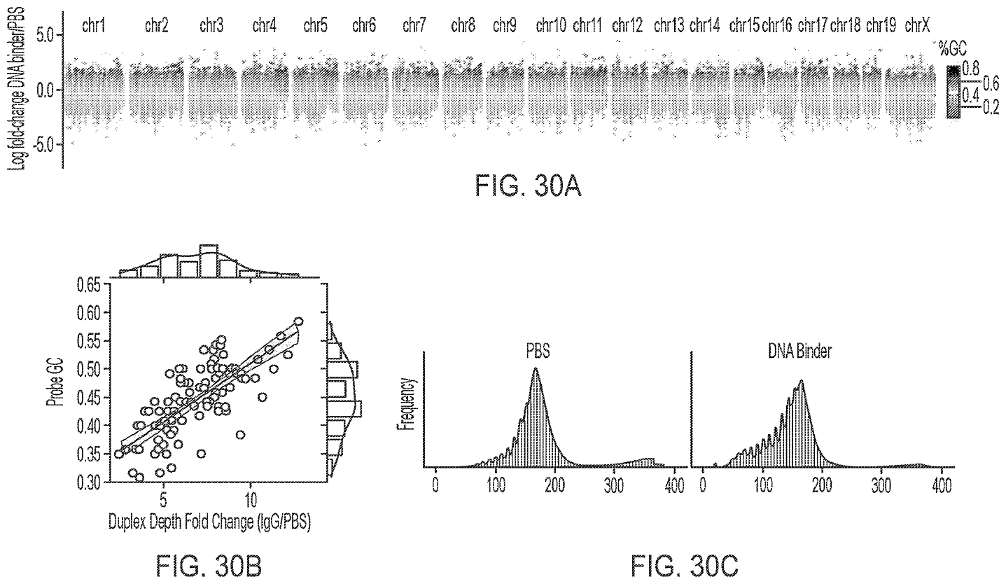

Upon liposome treatment at any dose, mice showed no signs of acute toxicity. When tumor-bearing mice were dosed with 300 mg/kg, DNA yields and tumor fractions began to shift towards the PBS baseline as early as 3 hours after liposome treatment (FIG. 26) suggesting that this effect is only transient and unlikely to cause notable toxicity.

### Discussion

A method has been developed that uses empty liposome nanoparticles to transiently inhibit the clearance of cfDNA from circulation and increase the amount of cfDNA that can be recovered from a blood draw. When tested in vitro, liposomes saturate the uptake pathways of macrophages, responsible for most cfDNA clearance in vivo, thereby decreasing their ability to uptake mononucleosomes. It was then demonstrated that in vivo liposome treatment decreases the clearance of mononucleosomes, as manifested by the prolonged plasma half-life of mononucleosomes in healthy mice. Finally, it was demonstrated that relative to PBS-treated mice, administration of decoy liposomes in a mouse tumor model increased cfDNA yields and enabled the recovery of significantly higher amounts of ctDNA molecules (50 mg/kg 2.9 fold, 100 mg/kg 6.1 fold, 200 mg/kg 6.9 fold and 300 mg/kg 9.1 fold) and the detection of ctDNA from more mutational sites (50 mg/kg 2.5 fold, 100 mg/kg 4.3 fold, 200 mg/kg 4.2 fold and 300 mg/kg 6.1 fold) from a blood draw. While the ability to detect more ctDNA molecules affords the possibility for a more sensitive and reliable ctDNA test, which can be especially important when the tumor signal is low and the sequencing error rate is high. The ability to detect ctDNA from more mutational sites offers higher resolution molecular profiling of tumors from a blood draw, which can be useful even in the setting of samples that already have cfDNA yields above the LOD of ctDNA testing. Cumulatively, these results hold great promise to continue to push beyond the current detection limits of liquid biopsies.

Cancer liquid biopsies that are based on the analysis of ctDNA from plasma offer many advantages to gather diagnostic information over tissue biopsies, as they can be obtained minimally invasively from patients. In addition to being convenient, low risk and rapid for the patient, liquid biopsies reflect signatures of disease across the entirety of the tumor burden while only a fraction of these signatures may be available through biopsy of a single region of one tumor as a result of tumor heterogeneity or be altogether inaccessible through surgery. In addition, the rapid clearance of cfDNA from blood means that ctDNA testing has the potential for real-time monitoring of disease and this highly desirable property has paved the way for ctDNA testing to be incorporated into clinical practice or to be tested in clinical trials for longitudinally monitoring the effectiveness of treatments, the emergence of resistance clones or the detection of relapses following curative-intent therapy in cancer patients.

Although the short half-life of cfDNA makes ctDNA a useful analyte for real-time monitoring of disease, it also makes it particularly challenging to achieve high levels of sensitivity for liquid biopsies when the burden of disease is low. Patients with low tumor burden, such as those with early stage or minimal residual disease, accumulate ultra-low levels of ctDNA in circulation which results in low tumor fractions. At such low tumor fractions, ctDNA sampling from a regular blood draw becomes stochastic and unreliable, as the likelihood of observing a given mutation is greatly diminished compared to that at higher tumor fractions.

To push beyond the current ctDNA detection limits and to improve the robustness of ctDNA testing, approaches to profile a higher mass of ctDNA in patients with low tumor fractions are needed. To date, most LOD improvements have taken place downstream of blood sampling through suppressing error rates, tracking more somatic variants or incorporating other signatures of disease such as methylation or fragmentation patterns. Despite their important contribution in driving LOD down in the setting of more accurately profiling existing ctDNA molecules in a blood draw, none of these can overcome the fundamental limitation of low tumor fractions resulting in a blood draw yielding no ctDNA molecules to be analyzed. To overcome this fundamental limitation, methods to sample more ctDNA are needed.

To sample more ctDNA, others have proposed to draw larger volumes of blood; but this may not always be desirable or possible in patients with advanced disease, or to plasmapherese patients' blood; which is invasive and costly and carries procedural risks to patients. Alternatively, over the last few years, others have attempted to increase the shedding of ctDNA by targeting barriers of ctDNA shedding from primary tumors; for instance, by using focused ultrasound combined with microbubbles to disrupt the blood brain barrier in glioblastoma patients² or by inducing cell death in primary tumors with stereotactic radiation¹. Increasing the shedding of ctDNA from primary tumors, artificially increases TFs and thus the mass of ctDNA recovered from a given volume of blood. However, the former approach is limited to brain tumors and the latter requires a priori knowledge of the tumor site and use of therapeutic doses of radiation, making them suboptimal in the context of cancer screening or monitoring of presumed disease-free patients.

This disclosure presents the first liquid biopsy contrast agent. This agent is able to overcome the fundamental limitation of low input ctDNA for liquid biopsies by safely and transiently inhibiting cfDNA clearance such that more ctDNA molecules can be recovered from a regular blood draw. Importantly, this approach does not require a priori knowledge of the tumor site, should generalize well across all shedding tumors as its mechanism of action is tumor-independent and does not require exposure to radiation. Furthermore, this contrast agent could also prove useful at increasing the sensitivity for other circulating biomarkers that may be subject to similar clearance routes beyond ctDNA, although this remains to be determined.

Methods

Liposome Synthesis

The liposomes were prepared with slight modifications from the protocol described by Saunders et al. using the lipid film re-hydration method. Briefly, Bovine Cholesterol (50 mol %) was solubilized in chloroform and added to SPE (50 mol %, cat) together with 1:1 (v/v) methanol. The solution was evaporated under nitrogen flow to form a thin dry film and vacuumed overnight to remove any traces of organic solvent. The lipid film was hydrated at 60° C. with the necessary volume of sterile DPB S, pHx to a total lipid concentration of 50 mg/ml. Finally, extrusion was performed at 60° C. with 1 μm and 0.4 μm polycarbonate membranes (cat, vendor), 21 and 20 times respectively, using an X extruder. For the fluorescent liposome used for biodistribution and clearance studies, 0.2 mol % of SPE was replaced for Cy7-SPE prior to solubilization with organic solvents.

Liposome Characterization

The liposomes were characterized using a Zetasizer NanoZS (Malvern Instrument). To measure the hydrodynamic diameter and polydispersity index (PdI) of the liposomes, samples were diluted 1:100 in PBS and analyzed. To measure the zeta potential of the liposomes, 10 μl of sample was added to 40 μl of PBS and 850 μl of deionized water.

NCP Extraction and Labelling

To prepare mononucleosomes for labelling, chromatin was extracted from CT26 cells using the Nucleosome Preparation Kit (#53504, Active Motif). The enzymatic digestion time for this cell line was optimized to 30 min. and the NCP preparation protocol was followed as per manufacturer's recommendations. To verify that chromatin had been successfully digested to mononucleosomes, the NCPs were subjected to DNA cleanup and digestion efficiency assessed via electrophoresis through a 1.5% agarose gel. To label NCPs, aliquots of 10 μg NCPs were first washed with PBS to remove any impurities from the NCP preparation reagents. 4 washes were performed using 30 kDa Amicon filters by centrifugation at 12000 rpm for 10 min. at 4° C. After concentrating the volume of the last wash to 40 μl, the protein yield was calculated using a commercial HeLa NCP standard by diluting the sample 10× and measuring absorbance at 230 nm using a Tecan. Sulfonated-Cy5 was added at a 25 dye: 1 protein molar ratio and the reaction performed at 4° C., 550 rpm overnight. Excess dye was removed using a packing column by centrifugation at 1000×g, 2 min. at room temperature. Labelling efficiency was measured by measuring Cy5 intensity against a Cy5 standard and protein yield by measuring absorbance at 230 nm. NCP labelling was confirmed by running a 4-12% Tris Glycine novex gel with unlabeled or Cy5-labelled NCPs (native running buffer, 4° C., 150 V, 90 min.) and confirming colocalization of a Cy5-positive band with a protein band after Coomassie staining corresponding to the NCP molecular weight.

Cell Lines

CT26 colon cancer cells (CRL-2638) and RAW264 macrophage cells (TIB-71) were purchased from the American Type Culture Collection. CT26 cells were cultured in RPMI (Gibco) and RAW264 cells in ATCC-formulated Dulbecco's Modified Eagle's Medium (DMEM) (Sigma). Both media were supplemented with 10% fetal bovine serum (Gibco) and 1% penicillin/streptomycin (Corning), and cells were cultured in a humidified atmosphere of 95% air and 5% CO₂ at 37° C. The CT26 cell line was declared pathogen free after being subjected to murine pathogen testing.

In Vitro Macrophage NCP Uptake Inhibition Assay

J774 and RAW264 cells were plated at a density of 30,000 and 45,000 cells/chamber in 8-well chamber slides (#80806, ibidi). Following overnight acclimatization, cells were incubated with 300 μl of liposomes diluted in DMEM at the desired concentrations (0.1-5 mg/ml) for 4 hours at 37° C. Next, 30 μl of NCPs were spiked into each well to achieve a final NCP concentration of 10 nM and further co-incubated liposomes and NCPs with cells for 2 hours at 37° C. to allow for NCP uptake. Cells incubated with DMEM for 4 hours followed by NCP were used as a positive control for uptake and cells incubated only with DMEM for 6 hours were used as a negative control. Cells were washed once with DMEM, stained with DAPI 1:2000 in DMEM for 10 min. and further washed (twice with DMEM and once with PBS) to remove any extracellular NCPs. Wells that were imaged were fixed with 4% PFA for 20 min. and washed with PBS prior to imaging on a MS2 Nikon microscope. To quantify cellular uptake, cells from 2 wells were pooled per condition. Cells were trypsinized with 50 μl of trypsin/well (5 min., 37° C.), trypsin quenched with 100 μl of DMEM and cells scraped off the well and resuspended in 1 ml of PBS. Cells were pelleted (350×g, 5 min., 25° C.) and fixed with 100 μl of 4% PFA (20 min., 25° C.). Cells were washed with 1 ml of FACS buffer (2% BSA in PBS) and resuspended in 400 μl of FACS buffer prior to analysis in a LSRII-HTS instrument. Excitation was performed with the 640 nm laser and emission measured with the APC-A filter. Gating was performed for cells, vertical and horizontal singlets and % positive cells calculated based on the median-APC value, thresholding for cells above the cutoff set using the positive control.

Plasma Collection

Blood was collected under anesthesia retro-orbitally for longitudinal studies and via terminal bleed at end point. For retro-orbital blood collections, 70 μL of blood were collected from alternating eyes with non-heparinized hematocrit capillary tubes and resuspended 1:1 (v/v) in 10 mM ethylenediaminetetraacetic acid (EDTA) in PBS. For terminal bleeds, mice were exsanguinated via cardiac puncture under anesthesia. Blood volume was measured and 10 mM EDTA added 1:1 (v/v). All blood samples were kept on ice and plasma separated within 2 hours of blood collection. Plasma was separated by centrifuging blood samples at 8,000×g for 5 min. at 4° C. and stored at −80° C. prior to Cy7 quantification, Widom601 quantification or ctDNA analysis with the MRD test¹⁶.

Liposome Biodistribution Study

100 mg/kg Cy7-liposomes (200 μl in PBS) were administered intravenously in awake mice. At 1 hour after administration, mice were euthanized and liver, spleen, lungs, kidneys and heart harvested (n=4). A PBS-treated mouse was used as a negative control to measure organ autofluorescence. Organ fluorescence was measured using the 800 nm filter of a Licor instrument. Biodistribution was quantified as a percent of total fluorescence across all organs for each mouse.

In Vivo NCP Pharmacokinetic Study

100 mg/kg Cy7-liposomes (200 μl in PBS) were administered intravenously in awake mice. 35 μL of blood were drawn from anesthetized mice retro-orbitally from alternating eyes longitudinally. Plasma was collected and Cy7 fluorescence in 20 μL of undiluted plasma samples measured using the 800 nm filter of a Licor instrument. Plasma fluorescence was quantified as total fluorescence per well for each sample. Percent plasma Cy7-liposome remaining was calculated by normalizing all samples to the first plasma sample collected 5 min. after the liposome injection. Time refers to minutes after the first blood sample was collected.

Tumor Models and Study Design

All animal work was approved by the committee on animal care (Massachusetts Institute of Technology (MIT), protocol 0420-023-23). To investigate how liposome dose affected the detection of ctDNA from blood, 4-6 weeks old female BALB/c mice (Taconic Biosciences) were injected subcutaneously with 2×10⁶ CT26 cells resuspended in Opti-Mem (Gibco) into bilateral rear flanks. Tumors were measured longitudinally over the next 2 weeks and mice randomized into different treatment groups (300 mg/kg, 200 mg/kg, 100 mg/kg, 50 mg/kg or PBS, n=5-7) at end point. As an internal control, blood was sampled retro-orbitally from each mouse prior to treatment. Subsequently, mice were injected with liposomes or PBS (10 μL/g at appropriate dose) via tail vein using a 0.3 mL insulin syringe. Blood was then sampled retro-orbitally 1 hour and 2 hours after treatment and the remainder of blood collected immediately after via cardiac puncture. Liver and spleen were also harvested and submitted for hematoxylin and eosin (H&E) staining.

Probe Panel

A CT26-specific probe panel was designed by selecting 100 heterozygous SNVs from a published list¹⁵.

cfDNA Extraction and Quantification

Frozen plasma was thawed and centrifuged at 15,000×g for 10 min. to remove residual cells and debris. 1×PBS was then added into plasma to make the total volume 2.1 ml for cfDNA extraction using the QIAsymphony Circulating DNA kit. The extracted cfDNA was quantified using a qPCR assay and then frozen at −20° C. until ready for further processing.

gDNA Extraction and Shearing

gDNA was extracted from CT26 cells or the buffy coat of the BALB/c strain using the QIAsymphony DNA Mini Kit. The extracted gDNA was sheared to 150 bp in size using a Covaris LE 220 instrument. Sheared DNA was quantified using Qubit (Qubit™ dsDNA HS, Invitrogen).

Library Construction, Hybrid Capture, and Sequencing

cfDNA and gDNA libraries were constructed using the Kapa Hyper Prep kit with custom dual index duplex UMI adapters (IDT)¹². A maximum of 50 μL of extracted cfDNA or 20 ng cfDNA mass was used as input into library construction. The prepared libraries were then quantified using the Quant-iT PicoGreen assay on a Hamilton STAR-line liquid handling system. Hybrid capture (HC) using a CT26 specific panel was performed using the xGen hybridization and wash kit with xGen Universal blockers (IDT) using a protocol adapted from Schmitt et al. For MRD tests, libraries were pooled up to 12-plex, with a library mass equivalent to 25 times DNA mass into LC for each sample, and 0.56 μmol/μL of a panel consisting of 120 bp long probes (IDT) targeting 100 CT26 specific mutations was applied. After the first round of HC, libraries were amplified by 16 cycles of PCR and then carried through a second HC but with half volumes of human Cot-1 DNA, xGen Universal blockers, and probes. Mouse Cot-1 DNA was also tested with no observed impact on assay performance. After the second round of HC, libraries were amplified through 8-16 cycles of PCR, quantified, and pooled for sequencing (151 bp paired-end runs) with a targeted raw depth of 250,000× per site for 20 ng DNA input. Sequencing data was processed by a duplex consensus calling pipeline as previously described¹².

### References for Example 5

1. Boniface, Christopher, Kathryn Baker, Chris Dieg, Carol Halsey, Ramtin Rahmani, Mark Deffenbach, Charles Thomas, Garth Tormoen, Nima Nabavizadeh, and Paul Spellman. “Abstract PROS: Radiation-assisted Amplification Sequencing (RAMP-Seq): Evaluating the use of stereotactic body radiation therapy (SBRT) for enriching circulating tumor DNA in liquid biopsies.” (2020): PROS-PROS.
2. Pacia, Christopher P., Jinyun Yuan, Yimei Yue, Lu Xu, Arash Nazeri, Rupen Desai, H. Michael Gach et al. “Sonobiopsy for minimally invasive, spatiotemporally-controlled, and sensitive detection of glioblastoma-derived circulating tumor DNA.” Theranostics 12, no. 1 (2022): 362.
3. Heitzer, Ellen, Imran S. Hague, Charles E S Roberts, and Michael R. Speicher. “Current and future perspectives of liquid biopsies in genomics-driven oncology.” Nature Reviews Genetics 20, no. 2 (2019): 71-88.
4. Lo, YM Dennis, Diana S C Han, Peiyong Jiang, and Rossa W K Chiu. “Epigenetics, fragmentomics, and topology of cell-free DNA in liquid biopsies.” Science 372, no. 6538 (2021).
5. Cescon, D. W., Bratman, S. V., Chan, S. M. & Siu, L. L. Circulating tumor DNA and liquid biopsy in oncology. Nat Cancer 1, 276-290 (2020).
6. Wan, Jonathan C M, Katrin Heider, Davina Gale, Suzanne Murphy, Eyal Fisher, Florent Mouliere, Andrea Ruiz-Valdepenas et al. “ctDNA monitoring using patient-specific sequencing and integration of variant reads.” Science translational medicine 12, no. 548 (2020).
7. Newman, Aaron M., Alexander F. Lovejoy, Daniel M. Klass, David M. Kurtz, Jacob J. Chabon, Florian Scherer, Henning Stehr et al. “Integrated digital error suppression for improved detection of circulating tumor DNA.” Nature biotechnology 34, no. 5 (2016): 547-555.
8. Kurtz, D. M., Soo, J., Co Ting Keh, L., Alig, S., Chabon, J. J., Sworder, B. J., Schultz, A., Jin, M. C., Scherer, F., Garofalo, A., Macaulay, C. W., Hamilton, E. G., Chen, B., Olsen, M., Schroers-Martin, J. G., Craig, A. F. M., Moding, E. J., Esfahani, M. S., Liu, C. L., . . . Alizadeh, A. A. (2021). Enhanced detection of minimal residual disease by targeted sequencing of phased variants in circulating tumor DNA. Nature Biotechnology, 39(12), 1537-1547.
9. Mitchell, Michael J., Margaret M. Billingsley, Rebecca M. Haley, Marissa E. Wechsler, Nicholas A. Peppas, and Robert Langer. “Engineering precision nanoparticles for drug delivery.” Nature Reviews Drug Discovery 20, no. 2 (2021): 101-124.
10. Gauthier, V. J., Tyler, L. N. & Mannik, M. Blood clearance kinetics and liver uptake of mononucleosomes in mice. J. Immunol. (1996) 156:1151-1156.
11. Kustanovich, A., Schwartz, R., Peretz, T. & Grinshpun, A. Life and death of circulating cell-free DNA. Cancer Biol. Ther. (2019) 20:1057-1067.
12. Parsons, H. A. et al. Sensitive Detection of Minimal Residual Disease in Patients Treated for Early-Stage Breast Cancer. Clin Cancer Res (2020). June 1; 26(11):2556-2564.
13. Saunders, N. R. M., et al. A Nanoprimer To Improve the Systemic Delivery of siRNA and mRNA. Nano Lett. (2020) June 10; 20(6):4264-4269.
14. Germain, M., et al. Priming the body to receive the therapeutic agent to redefine treatment benefit/risk profile. Sci Rep. (2018) March 19; 8(1):4797.
15. Castle J C, et al. Immunomic, genomic and transcriptomic characterization of CT26 colorectal carcinoma. BMC Genomics. (2014) March 13; 15(1):190.
16. Adalsteinsson, V. A., et al. Scalable whole-exome sequencing of cell-free DNA reveals high concordance with metastatic tumors. Nat Commun. (2017). November 6; 8(1):1324.
17. Lowary, P. T. and Widom J. New DNA sequence rules for high affinity binding to histone octamer and sequence-directed nucleosome positioning. J Mol Biol. (1998) February 13; 276(1):19-42.
18. Bettegowda, C. et al. Detection of circulating tumor DNA in early- and late-stage human malignancies. Sci. Transl. Med. 6, 224ra24 (2014).
19. Liu, M. C., G. R. Oxnard, E. A. Klein, C. S. M. C. C. Swanton, M. V. Seiden, Minetta C. Liu, Geoffrey R. Oxnard et al. “Sensitive and specific multi-cancer detection and localization using methylation signatures in cell-free DNA.” Annals of Oncology 31, no. 6 (2020): 745-759.
20. Parikh, A. R., Van Seventer, E. E., Siravegna, G., Hartwig, A. V., Jaimovich, A., He, Y., Kanter, K., Fish, M., Fosbenner, K. D., Miao, B., & Others. (2021). Minimal Residual Disease Detection using a Plasma-Only Circulating Tumor DNA Assay in Colorectal Cancer Patients. Clinical Cancer Research: An Official Journal of the American Association for Cancer Research.
21. Zhang, Y.-N., Poon, W., Tavares, A. J., McGilvray, I. D., & Chan, W. C. W. (2016). Nanoparticle—liver interactions: Cellular uptake and hepatobiliary elimination. Journal of Controlled Release: Official Journal of the Controlled Release Society, 240, 332-348.
22. Xiong, K., Shea, D., Rhoades, J., Blewett, T., Liu, R., Bae, J. H., Nguyen, E., Makrigiorgos, G. M., Golub, T. R., & Adalsteinsson, V. A. Duplex-Repair enables highly accurate sequencing, despite DNA damage. Nucleic Acids Research, 50(1), el. (2022)
23. Cutter, A. R. & Hayes, J. J. A brief review of nucleosome structure. FEBS Lett. 589, 2914-2922 (2015).

### Other Embodiments of the Present Disclosure

A. Method: A method for boosting the level of cfDNA with nanoparticles
B. Method: A method for assaying cfDNA that includes that step of boosting the level of cfDNA with nanoparticles
C. Kit: cfDNA-boosting nanoparticle agent, delivery vehicle, instructions (e.g., liquid biopsy kit)

A. A Method of Increasing the Concentration of Cell Free DNA (cfDNA)

A1. A method of increasing the concentration of cell free DNA (cfDNA), comprising administering to a subject an effective amount of a nanoparticle that results in an increased concentration of cfDNA in one or more biological fluids of the subject.

A2. The method of claim A1 wherein the concentration of cfDNA is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

A3. The method of claim A1 wherein between 0.1 and 10 quadrillion nanoparticles are administered to the subject.

A4. The method of claim A3 wherein 0.02 quadrillion nanoparticles per kilogram body weight are administered to the subject.

A5. The method of claim A1 wherein the one or more biological fluids are selected from: blood, plasma, serum, lymph, synovial fluid, interstitial fluid, cerebrospinal fluid, urine, mucus, and saliva.

A6. The method of claim A5 wherein the one or more biological fluids comprise blood.

A7. The method of claim A1 wherein the effective amount of the nanoparticle inhibits the activity of deoxyribonucleases in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the nanoparticle.

A8. The method of claim A1 wherein the effective amount of the nanoparticle prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the nanoparticle.

A9. The method of claim A1 wherein the nanoparticle comprises a naturally occurring lipid, a synthetic lipid, a naturally occurring polymer, or a synthetic polymer, or any combinations thereof.

A10. The method of claim A9 wherein the nanoparticle comprises at least one naturally occurring lipid.

All. The method of claim A9 wherein the nanoparticle comprises at least one natural polymer.

A12. The method of claim A9 wherein the nanoparticle comprises at least one synthetic lipid.

A13. The method of claim A9 wherein the nanoparticle comprises at least one synthetic polymer.

A14. The method of claim A9 wherein the nanoparticle is further conjugated to an inert material, optionally wherein the inert material is colloidal gold, silver, or platinum.

A15. The method of claim A9 wherein the nanoparticle is further conjugated to a deoxyribonuclease inhibitor.

A16. The method of claim A9 wherein the nanoparticle is further conjugated to a Kupffer cell targeting moiety.

A17. The method of claim A15 wherein the inhibitor is a DNase I inhibitor.

A18. The method of claim A1 wherein the nanoparticle has a size distribution from 5 nanometers to 1000 nanometers in diameter.

A19. The method of claim A1 wherein the nanoparticle has a circulatory half-life greater than 10 minutes.

A20. The method of claim A1 wherein the nanoparticle has a zeta potential from −60 mV to +60 mV.

A21. The method of claim A1 wherein the nanoparticle is administered intravenously.

A22. The method of claim A1 wherein the subject is a human patient.

A23. The method of claim A1 wherein the subject has, is suspected of having, or is at risk for a disease associated with the presence of cfDNA.

A24. The method of claim A23 wherein the disease is a cancer.

A25. The method of claim A24 wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

B. A Method for Assaying cfDNA from a Biological Fluid

B1. A method for assaying cfDNA from a biological fluid, said method comprising:
(a) administering to the subject an effective amount of a nanoparticle that results in an increased concentration of cfDNA in one or more biological fluids of the subject; and
(b) assaying the cfDNA.

B2. The method of claim B1, further comprising collecting the cfDNA from the one or more biological fluids before step (b).

B3. The method of claim B1 wherein the one or more biological fluids includes blood.

B4. The method of claim B1 wherein the effective amount of the nanoparticle inhibits the activity of deoxyribonucleases in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the nanoparticle.

B5. The method of claim B1 wherein the effective amount of the nanoparticle prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the nanoparticle.

B6. The method of claim B1 wherein the nanoparticle comprises a naturally occurring lipid, a synthetic lipid, a naturally occurring polymer, or a synthetic polymer, or any combinations thereof.

B7. The method of claim B6 wherein the nanoparticle comprises one or more naturally occurring lipid.

B8. The method of claim B6 wherein the nanoparticle comprises one or more natural polymer.

B9. The method of claim B6 wherein the nanoparticle comprises one or more synthetic lipids.

B10. The method of claim B6 wherein the nanoparticle comprises one or more synthetic polymer.

B11. The method of claim B6 wherein the nanoparticle is further conjugated to an inert material, optionally wherein the inert material is colloidal gold, silver, or platinum.

B12. The method of claim B6 wherein the nanoparticle is further conjugated to a deoxyribonuclease inhibitor.

B13. The method of claim B12 wherein the inhibitor is a DNase I inhibitor.

B14. The method of claim B6 wherein the nanoparticle is further conjugated to a Kupffer cell targeting moiety.

B15. The method of claim B1 wherein the nanoparticle has a size distribution from 5 nanometers to 1000 nanometers in diameter.

B16. The method of claim B1 wherein the nanoparticle has a circulatory half-life greater than 10 minutes.

B17. The method of claim B1 wherein the nanoparticle has a zeta potential from −60 mV to +60 mV.

B18. The method of claim B1 wherein the nanoparticle is administered intravenously.

B19. The method of claim B1 wherein the assaying of step (b) is conducted at least 1 hour, at least 2 hours, at least 3 hours, at least 4 hours, at least 5 hours, at least 10 hours, or at least 20 hours, but not longer than 25 hours, after administering step (a).

B20. The method of claim B1, wherein the assaying comprises sequencing the cfDNA.

B21. The method of claim B20 further comprising identifying the subject as having the disease if the cfDNA from the sample is determined to contain mutations that are indicative of the disease.

B22. The method of claim B21 wherein the disease is a cancer, optionally wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

C. A Kit for Increasing the Concentration of Cell Free DNA (cfDNA)

C1. A kit for increasing the concentration of cell free DNA (cfDNA) in a subject having, suspected of having or at risk for a disease associated with the presence of cfDNA from diseased tissue in one or more biological fluids, comprising:
(i) a nanoparticle sufficient for increasing the concentration of cfDNA in one or more biological fluids of the subject when administered to a subject;
(11) a pharmacologically acceptable fluid in which the nanoparticle is stored; and
(iii) instructions for administration of the kit.

C2. The kit of claim C1, wherein the kit is in the format of a liquid biopsy kit.

### INCORPORATION BY REFERENCE

All patents and other publications, including documents, references, published patents, published patent applications, and co-pending patent applications cited throughout this application are, for example, described in the technology described herein. For purposes of describing and disclosing the methodology described in such publications that may be used in connection with the above, expressly incorporated herein by reference. These publications are provided solely for their disclosure prior to the filing date of the present application. Nothing in this regard should be construed as an admission that the inventors have no right to advance the date of such disclosure, based on the prior invention or for any other reason. All statements regarding the date or content of these documents are based on information available to the Applicant and do not give any approval as to the accuracy of the date or content of these documents.

### EQUIVALENTS AND SCOPE

The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A method of increasing the concentration of cell-free DNA (cfDNA), comprising administering to a subject a therapeutically effective amount of a nanoparticle that results in an increased concentration of cfDNA in one or more biological fluids of the subject.

2. The method of claim 1, wherein the concentration of cfDNA is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

3. The method of any one of the previous claims, wherein between 0.1 and 10 quadrillion nanoparticles are administered to the subject.

4. The method of claim 3, wherein 0.02 quadrillion nanoparticles per kilogram body weight are administered to the subject.

5. The method of any one of the previous claims, wherein the one or more biological fluids are selected from: blood, plasma, serum, lymph, synovial fluid, interstitial fluid, cerebrospinal fluid, urine, mucus, and saliva.

6. The method of any one of the previous claims, wherein the one or more biological fluids comprise blood.

7. The method of any one of the previous claims, wherein the therapeutically effective amount of the nanoparticle inhibits the activity of deoxyribonucleases in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the nanoparticle.

8. The method of any one of the previous claims, wherein the therapeutically effective amount of the nanoparticle prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the nanoparticle.

9. The method of any one of the previous claims, wherein the nanoparticle comprises a naturally occurring lipid, a synthetic lipid, a naturally occurring polymer, or a synthetic polymer, or any combinations thereof.

10. The method of any one of the previous claims, wherein the nanoparticle comprises a naturally occurring or synthetic lipid selected from the following: phosphatidylglycerol (PG), phosphatidylcholine (PC), phosphatidylethanolamine (PEA), phosphatidylserine (PS), heptatriaconta-6,9,28,31-tetraen-19-yl 4-(dimethylamino)butanoate (DLin-MC3-DMA), cholesterol, 1,2-distearoyl-sn-glycero-3-phosphocholine (DSPC), 1,2-Distearoyl-sn-glycero-3-phosphoglycerol (DSPG), 1,2-dioleoyl-sn-glycero-3-phosphocholine (DOPC), 1,2-dierucoyl-sn-glycero-3-phosphocholine (DEPC), 1,2-dipalmitoyl-sn-glycero-3-phosphoglycerol (DPPG), 1,2-dioleoyl-sn-glycero-3-phosphoethanolamine (DOPE), 3-ethylphosphocholines (EPC), esterified propoxylated glycerol (EPG), 1,2-dimyristoyl-sn-glycero-3-phosphocholine (DMPC), 1,2-dioleoyl-3-trimethylammonium-propane (DOTAP), hydrogenated soy phosphatidylcholine (HSPC), and 1,2-dipalmitoyl-sn-glycero-3-phosphocholine (DPPC), or combinations thereof.

11. The method of any one of the previous claims, wherein the nanoparticle comprises a naturally occurring or synthetic polymer selected from the following: polyethylene glycol (PEG), poly-ε-caprolactone (PCL), poly(lactide-co-glycolide) (PLG), poly-L-lysine, alginate, a pollulan, and a cellulose, or combinations thereof.

12. The method of any one of the previous claims, wherein the nanoparticle further encapsulates or is conjugated to an inert material, optionally wherein the inert material is colloidal gold, silver, or platinum.

13. The method of any one of the previous claims, wherein the nanoparticle is further conjugated to a deoxyribonuclease inhibitor.

14. The method of claim 13, wherein the inhibitor is a DNase I inhibitor

15. The method of any one of the previous claims, wherein the nanoparticle is further conjugated to a Kupffer cell-targeting moiety.

16. The method of any one of the previous claims, wherein the nanoparticle has a size distribution from 5 nanometers to 1000 nanometers in diameter.

17. The method of any one of the previous claims, wherein the nanoparticle has a circulatory half-life greater than 10 minutes.

18. The method of any one of the previous claims, wherein the nanoparticle has a zeta potential from −70 mV to +70 mV.

19. The method of any one of the previous claims, wherein the nanoparticle is administered intravenously.

20. The method of any one of the previous claims, wherein the subject is a human patient.

21. The method of any one of the previous claims, wherein the subject has, is suspected of having, or is at risk for a disease associated with the presence of cfDNA.

22. The method of claim 21, wherein the disease is a cancer.

23. The method of claim 22, wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

24. A method for assaying cfDNA from a biological fluid, said method comprising:
(a) administering to the subject a therapeutically effective amount of a nanoparticle that results in an increased concentration of cfDNA in one or more biological fluids of the subject; and
(b) assaying the cfDNA.

25. The method of claim 24, further comprising collecting the cfDNA from the one or more biological fluids before step (b).

26. The method of any one of claims 24-25, wherein the one or more biological fluids includes blood.

27. The method of any one of claims 24-26, wherein the therapeutically effective amount of the nanoparticle inhibits the activity of deoxyribonucleases in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the nanoparticle.

28. The method of any one of claims 24-27, wherein the therapeutically effective amount of the nanoparticle prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the nanoparticle.

29. The method of any one of claims 24-28, wherein the nanoparticle comprises a naturally occurring lipid, a synthetic lipid, a naturally occurring polymer, or a synthetic polymer, or any combinations thereof.

30. The method of any one of claims 24-29, wherein the nanoparticle comprises a naturally occurring or synthetic lipid selected from the following: phosphatidylglycerol (PG), phosphatidylcholine (PC), phosphatidylethanolamine (PEA), phosphatidylserine (PS), heptatriaconta-6,9,28,31-tetraen-19-yl 4-(dimethylamino)butanoate (DLin-MC3-DMA), cholesterol, 1,2-distearoyl-sn-glycero-3-phosphocholine (DSPC), 1,2-Distearoyl-sn-glycero-3-phosphoglycerol (DSPG), 1,2-dioleoyl-sn-glycero-3-phosphocholine (DOPC), 1,2-dierucoyl-sn-glycero-3-phosphocholine (DEPC), 1,2-dipalmitoyl-sn-glycero-3-phosphoglycerol (DPPG), 1,2-dioleoyl-sn-glycero-3-phosphoethanolamine (DOPE), 3-ethylphosphocholines (EPC), esterified propoxylated glycerol (EPG), 1,2-dimyristoyl-sn-glycero-3-phosphocholine (DMPC), 1,2-dioleoyl-3-trimethylammonium-propane (DOTAP), hydrogenated soy phosphatidylcholine (HSPC), and 1,2-dipalmitoyl-sn-glycero-3-phosphocholine (DPPC), or combinations thereof.

31. The method of any one of claims 24-30, wherein the nanoparticle comprises a naturally occurring or synthetic polymer selected from the following: polyethylene glycol (PEG), poly-ε-caprolactone (PCL), poly(lactide-co-glycolide) (PLG), poly-L-lysine, alginate, a pollulan, and a cellulose, or combinations thereof.

32. The method of any one of claims 24-31, wherein the nanoparticle further encapsulates or is conjugated to an inert material, optionally wherein the inert material is colloidal gold, silver, or platinum.

33. The method of any one of claims 24-32, wherein the nanoparticle is further conjugated to a deoxyribonuclease inhibitor.

34. The method of claim 33, wherein the inhibitor is a DNase I inhibitor

35. The method of any one of claims 24-34, wherein the nanoparticle is further conjugated to a Kupffer cell-targeting moiety.

36. The method of any one of claims 24-35, wherein the nanoparticle has a size distribution from 5 nanometers to 1000 nanometers in diameter.

37. The method of any one of claims 24-36, wherein the nanoparticle has a circulatory half-life greater than 10 min.

38. The method of any one of claims 24-37, wherein the nanoparticle has a zeta potential from −70 mV to +70 mV.

39. The method of any one of claims 24-38, wherein the nanoparticle is administered intravenously.

40. The method of any one of claims 24-39, wherein the assaying of step (b) is conducted at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours, but not longer than 25 hours, after administering step (a).

41. The method of any one of claims 24-40, wherein the assaying of step (b) comprises sequencing the cfDNA.

42. The method of any one of claims 24-41, further comprising identifying the subject as having a disease if the cfDNA from the sample is determined to contain mutations that are indicative of the disease.

43. The method of claim 42, wherein the disease is a cancer, optionally wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

44. The method of claim 43, further comprising administering to the subject one or more treatments for cancer.

45. A kit for increasing the concentration of cell free DNA (cfDNA) in a subject having, suspected of having, or at risk for a disease associated with the presence of cfDNA from diseased tissue in one or more biological fluids, comprising:
(i) a nanoparticle sufficient for increasing the concentration of cfDNA in one or more biological fluids of the subject when administered to a subject;
(ii) a pharmacologically acceptable fluid in which the nanoparticle is stored; and
(iii) instructions for administration of the kit.

46. The kit of claim 45, wherein the kit is in the format of a liquid biopsy kit.

47. A method of increasing the concentration of cell-free DNA (cfDNA), comprising administering to a subject a therapeutically effective amount of an agent capable of binding cfDNA that results in an increased concentration of cfDNA in one or more biological fluids of the subject.

48. The method of claim 47, wherein the concentration of cfDNA is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

49. The method of claim 47 or claim 48, wherein the one or more biological fluids are selected from: blood, plasma, serum, lymph, synovial fluid, interstitial fluid, cerebrospinal fluid, urine, mucus, and saliva.

50. The method of claim 49, wherein the one or more biological fluids comprise blood or urine.

51. The method of any one of claims 47-50, wherein the therapeutically effective amount of the agent reduces the activity of deoxyribonucleases in the blood of the subject toward cfDNA by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the agent.

52. The method of any one of claims 47-51, wherein the therapeutically effective amount of the agent prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the agent.

51. The method of any one of claims 47-52, wherein the agent capable of binding cfDNA comprises protamine, a Sso7d homolog, and/or a monoclonal antibody specific for double stranded DNA (dsDNA).

52. The method of claim 51, wherein the protamine is in the form of protamine sulphate.

53. The method of claim 51, wherein the Sso7d homolog is a Sso7d protein from Sulfolobus solfataricus.

54. The method of claim 51, wherein the monoclonal antibody specific for dsDNA is a single domain antibody.

55. The method of any one of claims 47-54, wherein the agent is administered intravenously.

56. The method of any one of claims 47-55, wherein the subject is a human patient.

57. The method of any one of claims 47-56, wherein the subject has, is suspected of having, or is at risk for a disease associated with the presence of cfDNA.

58. The method of claim 57, wherein the disease is a cancer.

59. The method of claim 58, wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

60. A method of increasing the concentration of cell-free DNA (cfDNA), comprising administering to a subject a therapeutically effective amount of one or more of the following:
(a) a nanoparticle capable of binding cfDNA that results in an increased concentration of cfDNA in one or more biological fluids of the subject;
(b) an agent capable of binding cfDNA that results in an increased concentration of cfDNA in one or more biological fluids of the subject;
(c) an agent capable of inhibiting the activity of deoxyribonucleases that results in an increased concentration of cfDNA in one or more biological fluids of the subject; or
(d) any combination thereof comprising (a), (b), and/or (c).

61. The method of claim 60, wherein the concentration of cfDNA is increased by up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold.

62. The method of claim 60 or claim 61, wherein the one or more biological fluids are selected from: blood, plasma, serum, lymph, synovial fluid, interstitial fluid, cerebrospinal fluid, urine, mucus, and saliva.

63. The method of claim 62, wherein the one or more biological fluids comprise blood or urine.

64. The method of any one of claims 60-63, wherein the administration reduces the activity of deoxyribonucleases in the blood of the subject toward cfDNA by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of administration.

65. The method of any one of claims 60-64, wherein the administration prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the administration.

66. The method of any one of claims 60-65, wherein the nanoparticle is the nanoparticle specified by any one of claims 1-18.

67. The method of any one of claims 60-66, wherein the agent capable of binding cfDNA is the agent specified by any one of claims 47-53.

68. The method of any one of claims 60-67, wherein the agent capable of inhibiting deoxyribonucleases comprises a DNase I inhibitor.

69. The method of any one of claims 60-68, wherein the administration occurs intravenously.

70. The method of any one of claims 60-69, wherein the subject is a human patient.

71. The method of any one of claims 60-70, wherein the subject has, is suspected of having, or is at risk for a disease associated with the presence of cfDNA.

72. The method of claim 71, wherein the disease is a cancer.

73. The method of claim 72, wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

74. A method for assaying cfDNA from a biological fluid, said method comprising:
(a) administering to the subject a therapeutically effective amount of one or more of the following:
(i) a nanoparticle that results in an increased concentration of cfDNA in one or more biological fluids of the subject;
(ii) an agent capable of binding cfDNA that results in an increased concentration of cfDNA in one or more biological fluids of the subject;
(iii) an agent capable of inhibiting the activity of deoxyribonucleases that results in an increased concentration of cfDNA in one or more biological fluids of the subject; or
(iv) any combination thereof comprising (i), (ii), and/or (iii); and
(b) assaying the cfDNA.

75. The method of claim 74, further comprising collecting the cfDNA from the one or more biological fluids before step (b).

76. The method of claim 74 or claim 75, wherein the one or more biological fluids includes blood and/or urine.

77. The method of any one of claims 74-76, wherein the administration inhibits the activity of deoxyribonucleases in the blood of the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the activity of the deoxyribonucleases in the absence of the nanoparticle.

78. The method of any one of claims 74-77, wherein the administration prevents uptake of cfDNA by Kupffer cells in the subject by up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100%, as compared to the uptake of cfDNA by Kupffer cells in the absence of the nanoparticle.

79. The method of any one of claims 74-78, wherein the nanoparticle is the nanoparticle specified by any one of claims 1-18.

80. The method of any one of claims 74-79, wherein the agent capable of binding cfDNA is the agent specified by any one of claims 47-53.

81. The method of any one of claims 74-80, wherein the agent capable of inhibiting the activity of deoxyribonucleases comprises a DNase I inhibitor.

82. The method of any one of claims 74-81, wherein the administration occurs intravenously.

83. The method of any one of claims 74-82, wherein the assaying of step (b) is conducted at least 5 minutes, at least 10 minutes, at least 15 minutes, at least 30 minutes, at least 1 hour, at least 2 hours, at least 3 hours, at least 4 hours, at least 5 hours, at least 10 hours, or at least 20 hours, but not longer than 25 hours, after administering step (a).

84. The method of any one of claims 74-83, wherein the assaying of step (b) comprises sequencing the cfDNA.

85. The method of any one of claims 74-84, further comprising identifying the subject as having a disease if the cfDNA from the sample is determined to contain mutations that are indicative of the disease.

86. The method of claim 85, wherein the disease is a cancer, optionally wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

87. The method of claim 86, further comprising administering to the subject one or more treatments for cancer.

88. A diagnostic composition comprising one or more of the following:
(a) a nanoparticle that results in an increased concentration of cfDNA in one or more biological fluids of a subject when administered to the subject;
(b) an agent capable of binding cfDNA that results in an increased concentration of cfDNA in one or more biological fluids of a subject when administered to the subject;
(c) an agent capable of inhibiting the activity of deoxyribonucleases that results in an increased concentration of cfDNA in one or more biological fluids of a subject when administered to the subject; or
(d) any combination thereof comprising (a), (b), and/or (c); and
a pharmacologically acceptable excipient.

89. A kit for increasing the concentration of cell free DNA (cfDNA) in a subject having, suspected of having, or at risk for a disease associated with the presence of cfDNA from diseased tissue in one or more biological fluids, comprising:
(a) one or more agents selected from:
(i) a nanoparticle sufficient for increasing the concentration of cfDNA in one or more biological fluids of a subject when administered to the subject;
(ii) an agent capable of binding cfDNA sufficient for increasing the concentration of cfDNA in one or more biological fluids of the subject when administered to a subject;
(iii) an agent capable of inhibiting the activity of deoxyribonucleases sufficient for increasing the concentration of cfDNA in one or more biological fluids of the subject when administered to a subject; or
(iv) any combination thereof comprising (i), (ii), and/or (iii);
(b) a pharmacologically acceptable fluid in which the nanoparticle is stored; and
(c) instructions for administration of the kit.

90. The kit of claim 89, wherein the agent for binding cfDNA comprises protamine, a Sso7d homolog, and/or a monoclonal antibody specific for double stranded DNA (dsDNA).

91. The kit of claim 90, wherein the protamine is in the form of protamine sulphate.

92. The kit of claim 90, wherein the Sso7d homolog is a Sso7d protein from Sulfolobus solfataricus.

93. The kit of claim 90, wherein the monoclonal antibody specific for dsDNA is a single domain antibody.

94. The kit of any one of claims 89-93, wherein the kit is in the format of a liquid biopsy kit.
